(19) United States
(12) Patent Application Publication
(10) Pub. No.: US 2021/0164035 A1
(43) Pub. Date: Jun. 3, 2021

(54) METHODS AND DEVICES FOR SEQUENCING

(71) Applicant: Quantum-Si Incorporated, Guilford, CT (US)

(72) Inventors: Jonathan M. Rothberg, Guilford, CT (US); John H. Leamon, Stonington, CT (US); Jonathan C. Schultz, Guilford, CT (US); Michele Millham, Guilford, CT (US); Caixia Lv, Guilford, CT (US); Xiaxiao Ma, Branford, CT (US)

(73) Assignee: Quantum-Si Incorporated, Guilford, CT (US)

(21) Appl. No.: 17/082,226

(22) Filed: Oct. 28, 2020

Related U.S. Application Data
(60) Provisional application No. 63/101,214, filed on Oct. 29, 2019.

(51) Int. Cl. C12Q 1/6869; B01L 3/00

(52) U.S. Cl. CPC C12Q 1/6869; B01L 3/502715; B01L 2300/0816; C12Q 2533/107; C12Q 2561/113; C12Q 2535/101

(57) ABSTRACT

Methods and devices for enriching and analyzing target molecules from a sample are provided herein. In some embodiments, methods and devices involve enrichment of target molecules (e.g., using SCODA) and subsequent detection and analysis using sequencing.

### RELATED APPLICATIONS

This application claims the benefit under 35 U.S.C. § 119(e) of the filing date of U.S. Provisional Application Ser. No. 63/101,214, filed Oct. 29, 2019, the entire contents of which is incorporated herein by reference.

### BACKGROUND OF INVENTION

One mechanism for purifying, separating, or concentrating molecules of interest is called Synchronous Coefficient Of Drag Alteration (or “SCODA”) based purification. SCODA, known in some embodiments as scodaphoresis, is an approach that may be applied for purifying, separating, or concentrating particles.

SCODA based transport is used to produce net motion of a molecule of interest by synchronizing a time-varying driving force, which would otherwise impart zero net motion, with a time-varying drag (or mobility) alteration. If application of the driving force and periodic mobility alteration are appropriately coordinated, the result is net motion despite zero time-averaged forcing. With careful choice of both the temporal and spatial configuration of the driving and mobility altering fields, unique velocity fields can be generated, in particular a velocity field that has a non-zero divergence, such that this method of transport can be used for separation, purification and/or concentration of particles.

### SUMMARY OF INVENTION

Aspects of the instant disclosure provide methods, compositions, systems, and/or devices for use in a process to prepare a sample for analysis and/or analyze (e.g., analyze by sequencing) one or more target molecules in a sample. In some embodiments, a target molecule is a nucleic acid (e.g., DNA or RNA, including without limitation, cDNA, genomic DNA, mRNA, and derivatives and fragments thereof). In some embodiments, a target molecule is a protein or a polypeptide.

In some aspects, the disclosure provides a device for analyzing a target molecule from a biological sample, the device comprising an automated sample preparation module connected to a sequencing module, wherein the automated sample preparation module comprises a cartridge housing that is configured to receive a removable cartridge.

In some embodiments, the removable cartridge is a single-use cartridge or a multi-use cartridge. In some embodiments, the removable cartridge is configured to receive the biological sample. In some embodiments, the removable cartridge further comprises the biological sample. In some embodiments, the cartridge comprises one or more microfluidic channels configured to contain and/or transport a fluid used in a sample preparation process. In some embodiments, the cartridge comprises one or more affinity matrices, wherein each affinity matrix comprises an immobilized capture probe that has a binding affinity for the target molecule.

In some embodiments, the biological sample is a blood, saliva, sputum, feces, urine or buccal sample. A biological sample may be from a human, a non-human primate, a rodent, a dog, a cat, or a horse. In some embodiments, the biological sample comprises a bacterial cell or a population of bacterial cells.

In some embodiments, the target molecule is a target nucleic acid. In some embodiments, the target nucleic acid is a RNA or DNA molecule. In some embodiments, the target molecule is a target protein.

In some embodiments, the immobilized capture probe is an oligonucleotide capture probe, and wherein the oligonucleotide capture probe comprises a sequence that is at least partially complementary to the target nucleic acid. In some embodiments, the oligonucleotide capture probe comprises a sequence that is at least 80%, 90% 95%, or 100% complementary to the target nucleic acid. In some embodiments, the device or cartridge produces target nucleic acids with an average read-length for downstream sequencing applications that is longer than an average read-length produced using control methods.

In some embodiments, the immobilized capture probe is a protein capture probe that binds to the target protein. In some embodiments, the protein capture probe is an aptamer or an antibody. In some embodiments, the protein capture probe binds to the target protein with a binding affinity of 10⁻⁹ to 10⁻⁸ M, 10⁻⁸ to 10⁻⁷ M, 10⁻⁷ to 10⁻⁶ M, 10⁻⁶ to 10⁻⁵ M, 10⁻⁵ to 10⁻⁴ M, 10⁻⁴ to 10⁻³M, or 10⁻³ to 10⁻²M.

In some embodiments, the sequencing module performs nucleic acid sequencing. In some embodiments, the nucleic acid sequencing comprises single-molecule real-time sequencing, sequencing by synthesis, sequencing by ligation, nanopore sequencing, and/or Sanger sequencing.

In some embodiments, the device produces target nucleic acids with an average sequencing read-length that is longer than an average sequencing read-length produced using control methods.

In some embodiments, the sequencing module performs polypeptide sequencing. In some embodiments, the polypeptide sequencing comprises edman degradation or mass spectroscopy. In some embodiments, the sequencing module performs single-molecule polypeptide sequencing.

In some aspects, the disclosure provides a method of using a device of the disclosure, the method comprising: (i) lysing a biological sample in the sample preparation module; (ii) fragmenting the lysed sample of (i) in the sample preparation module; (iii) enriching the sample using an affinity matrix comprising an immobilized capture probe that has a binding affinity for the target molecule in the sample preparation module; (iv) moving the target molecule from the in the automated sample preparation module to the sequencing module; and (v) analyzing the target molecule in the sequencing module.

In some embodiments, step (i) comprises an electrolytic method, an enzymatic method, a detergent-based method, and/or mechanical homogenization. In some embodiments, step (i) comprises multiple lysis methods performed in series. The sample may be purified following lysis and prior to step (ii) or (iii) of a method for purifying a target molecule. In some embodiments, step (ii) comprises mechanical, chemical and/or enzymatic fragmentation methods. The sample may be purified following fragmentation and prior to step (iii). In some embodiments, step (iii) comprises enrichment using an electrophoretic method (e.g., affinity SCODA, FIGE, or PFGE). In some embodiments, step (iv) comprises moving the target molecule using microfluidics and/or a peristaltic pump. In some embodiments, step (v) comprises detection using absorbance, fluorescence, mass spectroscopy, and/or sequencing methods.

In addition to the exemplary aspects and embodiments described above, further aspects and embodiments will become apparent by reference to the drawings and by study of the following detailed descriptions.

### BRIEF DESCRIPTION OF DRAWINGS

Exemplary embodiments are illustrated in referenced figures of the drawings. The embodiments and figures disclosed herein are to be considered illustrative rather than restrictive.

FIG. 1 shows a plot of equation [10] showing the SCODA drift velocity in one dimension over the domain extending from −L to +L.

FIG. 2 shows a plot of equation [23] near the duplex melting temperature Tm illustrating the relative change in mobility as a function of temperature.

FIG. 3 shows a plot of mobility versus temperature for two different molecules with different binding energies to immobilized probe molecules. The mobility of the high binding energy target is shown by the curve on the right, while the mobility of the low binding energy target is shown by the curve on the left.

FIG. 4 shows the effect of an applied DC washing bias on molecules with two different binding energies. The solid curve represents the drift velocity of a target molecule with a lower binding energy to the bound probes than the molecules represented by the dashed curve.

FIG. 5 shows an example of an electric field pattern suitable for two dimensional SCODA based concentration in some embodiments. Voltages applied at electrodes A, B, C and D, are −V, 0, 0, and 0 respectively. Arrows represent the velocity of a negatively charged analyte molecule such as DNA. Color intensity represents electric field strength.

FIG. 6 shows stepwise rotation of the electric field leading to focusing of molecules whose mobility increases with temperature in one embodiment of affinity SCODA. A particle path is shown by the arrows.

FIG. 7 shows the gel geometry including boundary conditions and bulk gel properties used for electrothermal modeling.

FIG. 8 shows the results of an electrothermal model for a single step of the SCODA cycle in one embodiment. Voltage applied to the four electrodes was −120 V, 0 V, 0 V, 0 V. Spreader plate temperature was set to 55° C. (328 K).

FIG. 9 shows SCODA velocity vector plots in one exemplary embodiment of the invention.

FIGS. 10A and 10B show predictions of SCODA focusing under the application of a DC washing bias in one embodiment. FIG. 10A shows the SCODA velocity field for perfect match target. A circular spot indicates final focus location. FIG. 10B shows the SCODA velocity field for the single base mismatch target.

FIG. 11 shows the results of the measurement of temperature dependence of DNA target mobility through a gel containing immobilized complementary oligonucleotide probes for one exemplary separation.

FIG. 12 shows a time series of affinity SCODA focusing under the application of DC bias according to one embodiment. Perfect match DNA is tagged with 6-FAM (green) (leading bright line that focuses to a tight spot) and single base mismatch DNA is tagged with Cy5 (red) (trailing bright line that is washed from the gel). Images taken at 3 minute intervals. The first image was taken immediately following injection.

FIGS. 13A, 13B, 13C and 13D show the results of performing SCODA focusing with different concentrations of probes and in the presence or absence of 200 mM NaCl. Probe concentrations are 100 μM, 10 μM, 1 μM, and 100 μM, respectively. The buffer used in FIGS. 13A, 13B and 13C was 1×TB with 0.2 M NaCl. The buffer used in FIG. 13D was 1×TBE. Different amounts of target were injected in each of these experiments, and the camera gain was adjusted prevent saturation.

FIG. 14 shows an experiment providing an example of phase lag induced rotations. The field rotation is counterclockwise, that induces a clockwise rotation of the targets in the gel. Images were taken at 5 minute intervals.

FIG. 15A shows the focus location under bias for 250 bp and 1000 bp fragments labeled with different fluorescent markers, with squares indicating data for the application of a 10 V DC bias and circles indicating data for the application of a 20 V DC bias. FIG. 15B shows an image of the affinity gel at the end of the run, wherein images showing the location of each fluorescent marker have been superimposed.

FIGS. 16A and 16B show respectively the normalized fluorescence signal and the calculated rejection ratio of a 100 nucleotide sequence having a single base mismatch as compared with a target DNA molecule according to one example.

FIGS. 17A, 17B and 17C show enrichment of cDNA obtained from an EZH2 Y641N mutation from a mixture of wild type and mutant amplicons using affinity SCODA with the application of a DC bias. Images were taken at 0 minutes (FIG. 17A), 10 minutes (FIG. 17B), and 20 minutes (FIG. 17C).

FIG. 18 shows experimental results for the measurement of mobility versus temperature for methylated and unmethylated targets. Data points were fit to equation [23]. Data for the unmethylated target is fit to the curve on the left; data for the methylated target is fit to the curve on the right.

FIG. 19 shows the difference between the two mobility versus temperature curves which were fit to the data from FIG. 18. The maximum value of this difference is at 69.5° C., which is the temperature for maximum separation while performing affinity SCODA focusing with the application of a DC bias.

FIG. 20 shows experimental results for the separation of methylated (6-FAM, green) and unmethylated (Cy5, red) targets by using SCODA focusing with an applied DC bias.

FIGS. 21A-21D show the separation of differentially methylated oligonucleotides using affinity SCODA. FIGS. 21A and 21B show the results of an initial focus before washing unmethylated target from the gel for 10 pmol unmethylated DNA (FIG. 21A) and 0.1 pmol methylated DNA (FIG. 21B). FIGS. 21C and 21D show the results of a second focusing conducted after the unmethylated sequence had been washed from the gel for unmethylated and methylated target, respectively.

FIGS. 22A-22K show the results of the differential separation of two different sequences in the same affinity matrix using different oligonucleotide probes. FIG. 22A shows the gel after loading. FIGS. 22B and 22C show focusing at 55° C. after 2 minutes and 4 minutes, respectively. FIGS. 22D and 22E show focusing at 62° C. after 2 minutes and 4 minutes, respectively. FIGS. 22 F, 22G and 22H show focusing of the target molecules to an extraction well at the center of the gel after 0.5 minutes and 1 minute at 55° C. and at 3 minutes after raising the temperature to 62° C., respectively. FIGS. 22I, 22J and 22K show the application of a washing bias to the right at 55° C. after 6 minutes, 12 minutes and 18 minutes, respectively.

FIG. 23 shows an example method for preparing a target molecule from a biological sample (e.g., using an automated sample preparation module of the disclosure).

FIG. 24 shows a schematic diagram of a cross-section view of a cartridge 100 along the width of channels 102, in accordance with some embodiments.

FIG. 25 shows sequencing data output from DNA libraries generated with automated end-to-end (DNA extraction-to-finished library) sample preparation using a sample preparation device of the disclosure compared to libraries generated from manually extracted and purified DNA.

FIGS. 26A-26B show sequencing data output from a DNA library generated with automated end-to-end (DNA extraction-to-finished library) sample preparation using a sample preparation device of the disclosure compared to DNA libraries derived from samples that were size selected using commercial and manual methods.

### DETAILED DESCRIPTION OF INVENTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

As used herein, the term “differentially modified” means two molecules of the same kind that have been chemically modified in different ways. Non-limiting examples of differentially modified molecules include: a protein or a nucleic acid that has been methylated is differentially modified as compared with the unmethylated molecule; a nucleic acid that is hypermethylated or hypomethylated (e.g. as may occur in cancerous or precancerous cells) is differentially modified as compared with the nucleic acid in a healthy cell; a histone that is acetylated is differentially modified as compared with the non-acetylated histone; and the like.

In some embodiments, molecules that are differentially modified are identical to one another except for the presence of a chemical modification on one of the molecules. In some embodiments, molecules that are differentially modified are very similar to one another, but not identical. For example, where the molecules are nucleic acids or proteins, one of the biomolecules may share at least 95%, at least 96%, at least 97%, at least 98%, or at least 99% sequence identity with the differentially modified molecule.

### SCODA

SCODA can involve providing a time-varying driving field component that applies forces to particles in some medium in combination with a time-varying mobility-altering field component that affects the mobility of the particles in the medium. The mobility-altering field component is correlated with the driving field component so as to provide a time-averaged net motion of the particles. SCODA may be applied to cause selected particles to move toward a focus area.

In one embodiment of SCODA based purification, described herein as electrophoretic SCODA, time varying electric fields both provide a periodic driving force and alter the drag (or equivalently the mobility) of molecules that have a mobility in the medium that depends on electric field strength, e.g. nucleic acid molecules. For example, DNA molecules have a mobility that depends on the magnitude of an applied electric field while migrating through a sieving matrix such as agarose or polyacrylamide. By applying an appropriate periodic electric field pattern to a separation matrix (e.g. an agarose or polyacrylamide gel) a convergent velocity field can be generated for all molecules in the gel whose mobility depends on electric field. The field dependent mobility is a result of the interaction between a repeating DNA molecule and the sieving matrix, and is a general feature of charged molecules with high conformational entropy and high charge to mass ratios moving through sieving matrices. Since nucleic acids tend to be the only molecules present in most biological samples that have both a high conformational entropy and a high charge to mass ratio, electrophoretic SCODA based purification has been shown to be highly selective for nucleic acids.

The ability to detect specific biomolecules in a sample has wide application in the field of diagnosing and treating disease. Research continues to reveal a number of biomarkers that are associated with various disorders. Exemplary biomarkers include genetic mutations, the presence or absence of a specific protein, the elevated or reduced expression of a specific protein, elevated or reduced levels of a specific RNA, the presence of modified biomolecules, and the like. Biomarkers and methods for detecting biomarkers are potentially useful in the diagnosis, prognosis, and monitoring the treatment of various disorders, including cancer, disease, infection, organ failure and the like.

The differential modification of biomolecules in vivo is an important feature of many biological processes, including development and disease progression. One example of differential modification is DNA methylation. DNA methylation involves the addition of a methyl group to a nucleic acid. For example a methyl group may be added at the 5′ position on the pyrimidine ring in cytosine. Methylation of cytosine in CpG islands is commonly used in eukaryotes for long term regulation of gene expression. Aberrant methylation patterns have been implicated in many human diseases including cancer. DNA can also be methylated at the 6 nitrogen of the adenine purine ring.

Chemical modification of molecules, for example by methylation, acetylation or other chemical alteration, may alter the binding affinity of a target molecule and an agent that binds the target molecule. For example, methylation of cytosine residues increases the binding energy of hybridization relative to unmethylated duplexes. The effect is small. Previous studies report an increase in duplex melting temperature of around 0.7° C. per methylation site in a 16 nucleotide sequence when comparing duplexes with both strands unmethylated to duplexes with both strands methylated.

### Affinity SCODA

SCODAphoresis is a method for injecting biomolecules into a gel, and preferentially concentrating nucleic acids or other biomolecules of interest in the center of the gel. SCODA may be applied, for example, to DNA, RNA and other molecules. Following concentration, the purified molecules may be removed for further analysis. In one specific embodiment of SCODAphoresis-affinity SCODA-binding sites which are specific to the biomolecules of interest may be immobilized in the gel. In doing so one may be able generate a non-linear motive response to an electric field for biomolecules that bind to the specific binding sites. One specific application of affinity SCODA is sequence-specific SCODA. Here oligonucleotides may be immobilized in the gel allowing for the concentration of only DNA molecules which are complementary to the bound oligonucleotides. All other DNA molecules which are not complementary may focus weakly or not at all and can therefore be washed off the gel by the application of a small DC bias.

SCODA based transport is a general technique for moving particles through a medium by first applying a time-varying forcing (i.e. driving) field to induce periodic motion of the particles and superimposing on this forcing field a time-varying perturbing field that periodically alters the drag (or equivalently the mobility) of the particles (i.e. a mobility-altering field). Application of the mobility-altering field is coordinated with application of the forcing field such that the particles will move further during one part of the forcing cycle than in other parts of the forcing cycle. Specifically, the drift velocity υ(t) of a particle driven by an external force F(t) with a time varying drag coefficient ζ(t) (i.e. a varying mobility) is given by:

$$v ( t ) = \frac{F ( t )}{ζ ( t )} . \qquad [ 1 ]$$

If the external force and drag coefficient vary periodically such that

$$\begin{matrix}F ( t ) = {F}_{0} sin ( ω t ) & [ 2 ] \\ and , & \\ \frac{1}{ζ ( t )} = \frac{1}{{ζ}_{0}} + \frac{sin ( ω r + φ )}{{ζ}_{1}} & [ 3 ]\end{matrix}$$

then the drift velocity averaged over one complete cycle is given by:

$$\ddot{v} ( t ) = \frac{{F}_{0}}{2 {ζ}_{1}} cos ( φ ) \qquad [ 4 ]$$

By varying the drag (i.e. mobility) of the particle at the same frequency as the external applied force, a net drift can be induced with zero time-averaged forcing. The result of equation [4] can be used with an appropriate choice of driving force and drag coefficients that vary in time and space to generate a convergent velocity field in one or two dimensions. A time varying drag coefficient and driving force can be utilized in a real system to specifically concentrate (i.e. preferentially focus) only certain molecules, even where the differences between the target molecule and one or more non-target molecules are very small, e.g. molecules that are differentially modified at one or more locations, or nucleic acids differing in sequence at one or more bases.

### One Dimensional SCODA Concentration

By combining a spatially uniform driving force that varies periodically in time, with a drag coefficient that varies in time as well as in space it is possible to generate a convergent velocity field in one dimension. Consider the case of a charged particle with mobility μ moving under the influence of an applied electric field E; its velocity will be given by:

υ(x,t)=μ(x,t)E(x,t) [5]

If electric field is varied periodically in time such that:

E(x,t)=E₀ sin(wt) [6]

and a linear mobility gradient is provided within the domain −L.ltoreq.x.ltoreq.L that varies at the same period:

μ(x,t)=μ₀+(kx)sin(ωt+π) [7]

where k can be thought of as the amplitude of the mobility variation, SCODA-based separation of particles can be achieved.

There are a number of ways to establish a mobility gradient for charged molecules moving in solution under the influence of an applied external electric field. For example, a time-varying electric field may be provided as described above, a temperature gradient may be established, a pH gradient may be established, a light gradient may be established for molecules which undergo a conformational change in the presence or absence of light, or the like.

With the mobility gradient of equation [7] provided, the velocity becomes:

υ(x,t)=[μ₀+(kx)sin(ωt+ϕ)][E₀ sin(ωt)] [8].

Taking the time average of this velocity over one complete cycle yields the following drift velocity:

$$\begin{matrix}{\overline{v}}_{d} ( x , t ) = \frac{ω}{2 π} {\int }_{0}^{\frac{2 π}{ω}} v ( x , t ) dt & [ 9 ] \\ {\overline{v}}_{d} ( x , t ) = \frac{kx}{2} {E}_{0} cos ( φ ) . & [ 10 ]\end{matrix}$$

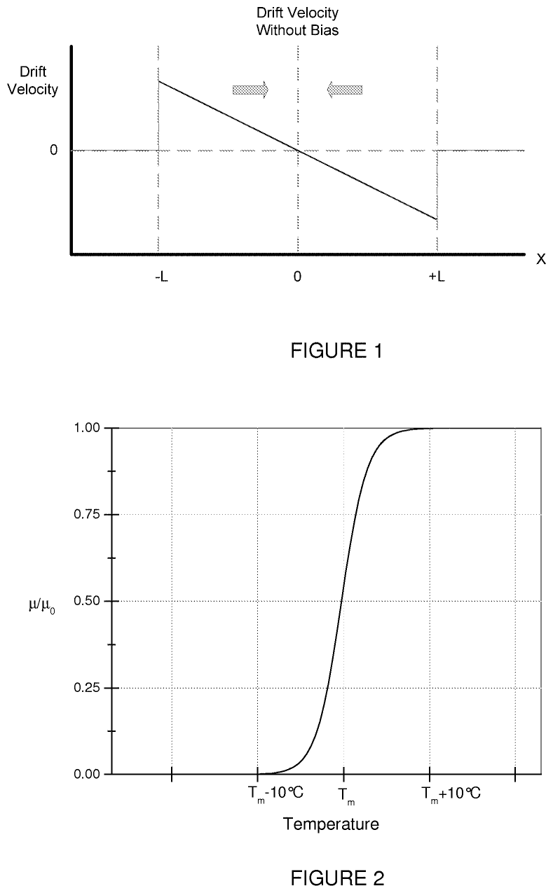

This velocity field has an equilibrium point at x=0 and can be made convergent or divergent depending on the sign of kE₀ cos(ϕ). For positive values the velocity field is divergent and for negative values it is convergent. FIG. 1 shows the velocity plotted as a function of x for the case where kE₀ cos(ϕ)<0. The arrows in FIG. 1 indicate the direction of drift. All particles between −L and +L will drift towards the zero velocity point at x=0. Outside of the domain the time averaged velocity is zero as the mobility is only altered between −L and +L.

In the embodiment illustrated in FIG. 1, the velocity takes on a positive value for negative values of x and vice versa for positive values of x resulting in all particles within the domain drifting towards x=0 where the velocity is zero.

### Two Dimensional SCODA

To extend the result of equation [10] to two dimensions, in some embodiments a rotating electric field is used as the driving field and a rotating mobility gradient is established:

\vec{E}=E₀ cos(ωt)\vec{i}−E₀ sin(ωt)\vec{j} [11]

μ=μ₀+k[x cos(ωt+ϕ)−y sin(ωt+ϕ)] [12].

As in the one dimensional case \vec{υ}=μ \vec{E}, and the same integration as in equation [9] can be performed to yield the time averaged drift velocity in two dimensions:

$$\begin{matrix}{\ddot{v}}_{x} = \frac{ω}{2 π} {\int }_{0}^{\frac{2 π}{ω}} {E}_{0} cos ( ω t ) ( {μ}_{0} + k ( x cos ( ω t + φ ) - y sin ( ω t + φ ) ) ) dt & [ 13 ] \\ {\overline{v}}_{y} = \frac{ω}{2 π} {\int }_{0}^{\frac{2 π}{ω}} - {E}_{0} cos ( ω t ) ( {μ}_{0} + k ( x cos ( ω t + φ ) - y sin ( ω t + φ ) ) ) dt . & [ 14 ]\end{matrix}$$

This results in the following expression for the drift velocity:

$$\vec{v} = \frac{{E}_{0} k}{2} ( ( x cos ( φ ) - y sin ( φ ) ) \hat{i} + ( x sin ( φ ) + y cos ( φ ) ) \hat{j} ) . \qquad [ 15 ]$$

Rewriting in polar coordinates and simplifying yields:

$$\vec{v} = \frac{{E}_{0} kr}{2} ( cos ( φ ) r ++ sin ( φ ) \hat{θ} ) . \qquad [ 16 ]$$

This result highlights a number of aspects of SCODA in two dimensions. It shows that despite the zero time averaged forcing there will be non-zero drift everywhere except at the point in the medium where r=0. It shows that the nature of the drift depends on the relative phase, ϕ, of the two signals, with the strength of focusing (the radial, \hat{r}, term) being proportional to the cosine of the phase lag between the electric driving field oscillations and the mobility oscillations. For a 0° phase angle there is a purely focusing velocity field with net drift directed towards the center of the domain. For a 180° phase angle the velocity field is pure de-focusing with net drift away from the center of the gel. And for phase angles of 90° and 270° the velocity field is purely rotational. At intermediate angles the resultant velocity field will be a combination of both rotational and focusing components. To achieve efficient focusing, in some embodiments the phase difference between the driving force and the mobility variation is as small as possible.

### Generation of a Time Varying Mobility Field

Previous applications of SCODA based concentration used the fact that the mobility of DNA in a sieving matrix such as agarose or polyacrylamide depends on the magnitude of the applied electric field. In some applications, the molecules of interest may have a mobility that does not normally depend strongly on electric field, such as short nucleic acids less than 200 bases, biomolecules other than nucleic acids (e.g. proteins or polypeptides), or the like. In some applications, it may be desired to purify only a subset of the nucleic acids in a sample, for example purifying or detecting a single gene from a sample of genomic DNA or purifying or detecting a chemically modified molecule (e.g. methylated DNA) from a differentially modified molecule having the same basic structure (e.g. unmethylated DNA having the same sequence), or the like.

SCODA-based purification of molecules that do not have a mobility that is strongly dependent on electrical field strength (i.e. which have a low value of k based on variations in electric field strength) can be achieved by using a SCODA matrix that has an affinity to the molecule to be concentrated. An affinity matrix can be generated by immobilizing an agent with a binding affinity to the target molecule (i.e. a probe) in a medium. Using such a matrix, operating conditions can be selected where the target molecules transiently bind to the affinity matrix with the effect of reducing the overall mobility of the target molecule as it migrates through the affinity matrix. The strength of these transient interactions is varied over time, which has the effect of altering the mobility of the target molecule of interest. SCODA drift can therefore be generated. This technique is called affinity SCODA, and is generally applicable to any target molecule that has an affinity to a matrix.

Affinity SCODA can selectively enrich for nucleic acids based on sequence content, with single nucleotide resolution. In addition, affinity S CODA can lead to different values of k for molecules with identical DNA sequences but subtly different chemical modifications such as methylation. Affinity SCODA can therefore be used to enrich for (i.e. preferentially focus) molecules that differ subtly in binding energy to a given probe, and specifically can be used to enrich for methylated, unmethylated, hypermethylated, or hypomethylated sequences.

Exemplary media that can be used to carry out affinity SCODA include any medium through which the molecules of interest can move, and in which an affinity agent can be immobilized to provide an affinity matrix. In some embodiments, polymeric gels including polyacrylamide gels, agarose gels, and the like are used. In some embodiments, microfabricated/microfluidic matrices are used.

Exemplary operating conditions that can be varied to provide a mobility altering field include temperature, pH, salinity, concentration of denaturants, concentration of catalysts, application of an electric field to physically pull duplexes apart, or the like.

Exemplary affinity agents that can be immobilized on the matrix to provide an affinity matrix include nucleic acids having a sequence complementary to a nucleic acid sequence of interest, proteins having different binding affinities for differentially modified molecules, antibodies specific for modified or unmodified molecules, nucleic acid aptamers specific for modified or unmodified molecules, other molecules or chemical agents that preferentially bind to modified or unmodified molecules, or the like.

The affinity agent may be immobilized within the medium in any suitable manner. For example where the affinity agent is an oligonucleotide, the oligonucleotide may be covalently bound to the medium, acrydite modified oligonucleotides may be incorporated directly into a polyacrylamide gel, the oligonucleotide may be covalently bound to a bead or other construct that is physically entrained within the medium, or the like.

Where the affinity agent is a protein or antibody, in some embodiments the protein may be physically entrained within the medium (e.g. the protein may be cast directly into an agarose or polyacrylamide gel), covalently coupled to the medium (e.g. through use of cyanogen bromide to couple the protein to an agarose gel), covalently coupled to a bead that is entrained within the medium, bound to a second affinity agent that is directly coupled to the medium or to beads entrained within the medium (e.g. a hexahistidine tag bound to NTA-agarose), or the like.

Where the affinity agent is a protein, the conditions under which the affinity matrix is prepared and the conditions under which the sample is loaded should be controlled so as not to denature the protein (e.g. the temperature should be maintained below a level that would be likely to denature the protein, and the concentration of any denaturing agents in the sample or in the buffer used to prepare the medium or conduct SCODA focusing should be maintained below a level that would be likely to denature the protein).

Where the affinity agent is a small molecule that interacts with the molecule of interest, the affinity agent may be covalently coupled to the medium in any suitable manner.

One exemplary embodiment of affinity SCODA is sequence-specific SCODA. In sequence specific SCODA, the target molecule is or comprises a nucleic acid molecule having a specific sequence, and the affinity matrix contains immobilized oligonucleotide probes that are complementary to the target nucleic acid molecule. In some embodiments, sequence specific SCODA is used both to separate a specific nucleic acid sequence from a sample, and to separate and/or detect whether that specific nucleic acid sequence is differentially modified within the sample. In some such embodiments, affinity SCODA is conducted under conditions such that both the nucleic acid sequence and the differentially modified nucleic acid sequence are concentrated by the application of SCODA fields. Contaminating molecules, including nucleic acids having undesired sequences, can be washed out of the affinity matrix during SCODA focusing. A washing bias can then be applied in conjunction with SCODA focusing fields to separate the differentially modified nucleic acid molecules as described below by preferentially focusing the molecule with a higher binding energy to the immobilized oligonucleotide probe.

### Mobility of a Target in an Affinity Matrix

The interactions between a target and immobilized probes in an affinity matrix can be described by first order reaction kinetics:

$$[ T ] + [ P ] {{⇌}_{{k}_{r}}}^{{k}_{f}} [ T … P ] . \qquad [ 17 ]$$

Here [T] is the target, [P] the immobilized probe, [T. P] the probe-target duplex, k_{f} is the forward (hybridization) reaction rate, and kᵣ the reverse (dissociation) reaction rate. Since the mobility of the target is zero while it is bound to the matrix, the effective mobility of the target will be reduced by the relative amount of target that is immobilized on the matrix:

$${μ}_{effective} = {μ}_{0} \frac{[ T ]}{[ T ] + [ T … P ]} \qquad [ 18 ]$$

where μ₀ is the mobility of the unbound target. Using reasonable estimates for the forward reaction rate⁶ and an immobilized probe concentration that is significantly higher than the concentration of the unbound target, it can be assumed that the time constant for hybridization should be significantly less than one second. If the period of the mobility-altering field is maintained at longer than one second, it can be assumed for the purposes of analysis that the binding kinetics are fast and equation [17] can be rewritten in terms of reaction rates:

$$\begin{matrix}{k}_{f} [ T ] [ P ] = {k}_{r} [ T … P ] & [ 19 ] \\ [ T ] = \frac{{k}_{r}}{{k}_{f}} \frac{[ T … P ]}{[ P ]} . & [ 20 ]\end{matrix}$$

Inserting [20] into equation [18] and simplifying yields:

$${μ}_{effective} = {μ}_{0} \frac{1}{1 + \frac{{k}_{f}}{{k}_{r}} [ P ]} . \qquad [ 21 ]$$

From this result it can be seen that the mobility can be altered by modifying either the forward or reverse reaction rates. Modification of the forward or reverse reaction rates can be achieved in a number of different ways, for example by adjusting the temperature, salinity, pH, concentration of denaturants, concentration of catalysts, by physically pulling duplexes apart with an external electric field, or the like. In one exemplary embodiment described in greater detail below, the mechanism for modifying the mobility of target molecules moving through an affinity matrix is control of the matrix temperature.

To facilitate analysis, it is helpful to make some simplifying assumptions. First it is assumed that there are a large number of immobilized probes relative to target molecules. So long as this is true, then even if a large fraction of the target molecules become bound to the probes the concentration of free probes, [P], will not change much and it can be assumed that [P] is constant. Also, it is assumed that the forward reaction rate k_{f} does not depend on temperature. This not strictly true, as the forward reaction rate does depend on temperature. Secondary structure in the immobilized probe or in the target molecule can result in a temperature dependent forward reaction rate. However, in embodiments operating at a temperature range near the duplex melting temperature the reverse reaction rate has an exponential dependence on temperature and the forward reaction rate has a much weaker temperature dependence, varying by about 30% over a range of 30° C. around the melting temperature. It is additionally assumed that the target sequence is free of any significant secondary structure. Although this final assumption would not always be correct, it simplifies this initial analysis.

To determine the temperature dependence of the reverse reaction rate, an Arrhenius model for unbinding kinetics is assumed. This assumption is justified by recent work in nanopore force spectroscopy.

$${k}_{r} = {Ae}^{\frac{Δ G}{{k}_{b} T}} . \qquad [ 22 ]$$

Here A is an empirically derived constant, ΔG is the probe-target binding energy, k_{b} is the Boltzmann constant, and T the temperature. Inserting this into [21], rewriting the free energy ΔG as ΔH-TΔS, and collecting constant terms allows the mobility to be rewritten as:

$${μ}_{effective} = {μ}_{0} \frac{1}{1 + β {e}^{\frac{- Δ H + T Δ S}{{k}_{b} T}}} . \qquad [ 23 ]$$

Equation [23] describes a sigmoidal mobility temperature dependence. The shape of this curve is shown in FIG. 2. At low temperature the mobility is nearly zero. This is the regime where thermal excitations are insufficient to drive target molecules off of the affinity matrix. At high temperature target molecules move at the unbound mobility, where the thermal energy is greater than the binding energy. Between these two extremes there exists a temperature range within which a small change in temperature results in a large change in mobility. This is the operating regime for embodiments of affinity SCODA that utilize temperature as the mobility altering parameter.

In embodiments of affinity SCODA used to separate nucleic acids based on sequence, i.e. sequence-specific SCODA, this temperature range tends to lie near the melting temperature of the probe-target duplex. Equations [10] and [16] state that the speed of concentration is proportional to k, which is a measure of how much the mobility changes during one SCODA cycle. Operating near the probe-target duplex melting temperature, where the slope of the mobility versus temperature curve is steepest, maximizes k for a given temperature swing during a SCODA cycle in embodiments where temperature is used as the mobility altering parameter.

In some embodiments, affinity SCODA may be conducted within a temperature gradient that has a maximum amplitude during application of SCODA focusing fields that varies within about ±20° C., within about ±10° C., within about ±5° C., or within about ±2° C. of the melting temperature of the target molecule and the affinity agent.

It is possible to describe affinity SCODA in one dimension by replacing the time dependent mobility of equation [7] with the temperature dependent mobility of equation [23] and a time dependent temperature:

$$T ( x , t ) = {T}_{m} + {T}_{a} ( \frac{x}{L} ) sin ( ω t + φ ) . \qquad [ 24 ]$$

Here, the temperature oscillates around Tₘ, the probe target melting temperature, and Tₐ is the maximum amplitude of the temperature oscillations at x=±L. To get an analytical expression for the drift velocity, υ_{d}=μE, as a function of temperature, a Taylor expansion of equation [23] is performed around Tₘ:

$${μ}_{effective} = μ ( {T}_{m} ) - \frac{{μ}_{0} βΔ {He}^{\frac{- Δ H + T Δ S}{{k}_{b} {T}_{m}}}}{{k}_{b} {{T}_{m}^{2} ( 1 + β {e}^{\frac{- Δ H + T Δ S}{{k}_{b} {T}_{m}}} )}^{2}} ( T - {T}_{m} ) + O ( {( T - {T}_{m} )}^{2} ) . \qquad [ 25 ]$$

which can be rewritten as:

μ_{effective}=μ(Tₘ)+α(T−Tₘ)+O((T−Tₘ)²) [26].

Here the first term in the Taylor expansion has been collected into the constant α. Combining [24] and [26] into an expression for the mobility yields an expression similar to [7]:

$$μ ( t ) = μ ( {T}_{m} ) + ( \frac{α {T}_{α} x}{L} ) sin ( ω t + φ ) . \qquad [ 27 ]$$

Equation [27] can be used to determine the time averaged drift velocity for both the one dimensional and two dimensional cases by simply replacing k with:

$$α \frac{{T}_{α}}{L} = \frac{{μ}_{0} β Δ {He}^{\frac{- Δ H + T Δ S}{{k}_{b} {T}_{m}}}}{{k}_{b} {{T}_{m}^{2} ( 1 + β {e}^{\frac{- Δ H + T Δ S}{{k}_{b} {T}_{m}}} )}^{2}} ( \frac{{T}_{α}}{L} ) . \qquad [ 28 ]$$

The drift velocity is then given by:

$${\overline{v}}_{d} ( x , t ) = \frac{α {T}_{α} x}{2 L} {E}_{0} cos ( φ ) \qquad [ 29 ]$$

in one dimension, and:

$$\overline{v} = \frac{{E}_{0} α {T}_{α} r}{2 L} ( cos ( φ ) \hat{r} + sin ( φ ) \hat{θ} ) . \qquad [ 30 ]$$

in two dimensions. This result shows that if a two dimensional gel functionalized with immobilized probes (i.e. an affinity matrix), then by combining a rotating temperature gradient with a rotating dipole electric field, all target molecules should be forced towards a central region in the gel, thus concentrating a target molecule that binds to the immobilized probes.
Molecular Separation with Affinity SCODA

In some embodiments, affinity SCODA is used to separate two similar molecules (e.g. the same molecule that has been differentially modified, or which differs in sequence at only one or a few locations) with differing binding affinities for the immobilized probe. Beginning with two molecular species, each with a different binding energy to the immobilized probes, these two molecular species can be separated by superimposing a washing motive force over the driving and mobility altering fields used to produce SCODA focusing, to provide net motion of molecules that have a lesser binding affinity for the immobilized probe (i.e. the molecules that have a higher binding affinity for the immobilized probe are preferentially focused during the application of the SCODA focusing fields). In some embodiments, the washing force is a small applied DC force, referred to herein as a DC bias.

In the one dimensional case when a small DC force is applied as a washing or bias force, the electric field becomes:

E(x,t)=E₀ sin(ωt)+E_{b} [31]

where E_{b} is the applied DC bias. The final drift velocity has superimposed on the SCODA focusing velocity a constant velocity proportional to the strength of the bias field:

$${\overline{v}}_{d} ( x , t ) = \frac{α {T}_{α} x}{2 L} {E}_{0} cos ( φ ) + μ ( {T}_{m} ) {E}_{b} . \qquad [ 32 ]$$

This drift velocity will tend to move the final focus location either to the left or right depending on the direction of bias. The amount by which this bias moves a focus off center depends on the strength of the interaction between the target and probe molecules. The differential strength of the target-probe interaction can therefore serve as a mechanism to enable molecular separation of two highly similar species.

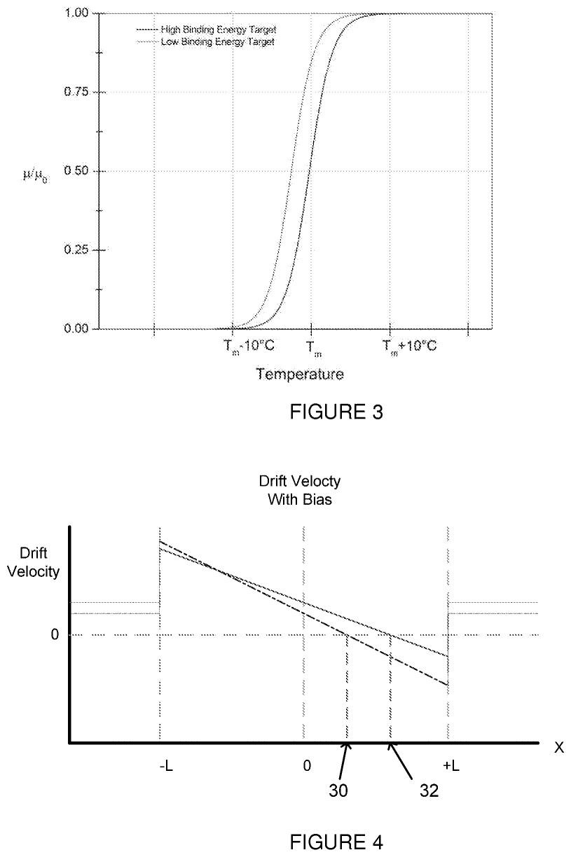

Consider two molecules that have different binding affinities for an immobilized probe. Reducing the probe-target binding energy, ΔG in equation [23], will serve to shift the mobility versus temperature curve to the left on the temperature scale as shown in FIG. 3. The mobility of the high binding energy target is shown by the curve on the right, while the mobility of the low binding energy target is shown by the curve on the left.

If the SCODA system in this exemplary embodiment is operated at the optimal focusing temperature for the higher binding energy molecule, Tₘ in FIG. 3, then the mobility of the lower binding energy molecule will be higher and will have weaker temperature dependence. In terms of equation [32] the molecule with lower binding energy will have a larger value of μ(Tₘ) and a smaller value of a. This means that a lower binding energy molecule will have a lower SCODA drift velocity and a higher velocity under DC bias, resulting in a different final focus location than the high binding energy molecule as illustrated in FIG. 4.

FIG. 4 shows the effect of an applied DC bias on molecules with two different binding energies for the immobilized probe according to one embodiment. The solid curve represents the drift velocity of a target molecule with a lower binding energy to the bound probes than the molecules represented by the dashed curve. The final focus location is the point where the drift velocity is equal to zero. The molecules represented by the solid curve have both a lower SCODA drift velocity and a higher DC velocity compared to the molecules represented by the dashed curve. When SCODA focusing is combined with a DC bias the lower binding energy molecules will focus further away from the unbiased focus at x=0, resulting in two separate foci, one for each molecular species. The final focus position for the high binding energy molecule is indicated by reference numeral 30. The final focus position for the low binding energy molecule is indicated by reference numeral 32.

The two dimensional case is the same as the one dimensional case, the superimposed velocity from the applied washing bias moves the final focus spot off center in the direction of the washing bias.

In some embodiments, if the difference in binding energies between the molecules to be separated is large enough and a sufficiently high washing bias is applied, the low binding energy molecules can be washed off of the affinity matrix while molecules with higher binding energy are retained in the affinity matrix, and may be captured at a focus location within the affinity matrix (i.e. preferentially focused) through the application of SCODA focusing fields.

### Generation of a Time Varying Temperature Gradient

Embodiments of affinity SCODA that use variations in temperature as the mobility altering field may use a periodically varying temperature gradient to produce a convergent velocity field. A periodically varying temperature gradient may be provided in any suitable manner, for example by the use of heaters or thermoelectric chillers to periodically heat and cool regions of the medium, the use of radiative heating to periodically heat regions of the medium, the application of light or radiation to periodically heat regions of the medium, Joule heating using the application of an electric field to the medium, or the like.

A periodically varying temperature gradient can be established in any suitable manner so that particles that are spaced a farther distance from a desired focus spot experience greater mobility (i.e. are at a higher temperature and hence travel farther) during times of application of the driving field towards the desired focus spot than during times of application of the driving field away from the desired focus spot. In some embodiments, the temperature gradient is rotated to produce a convergent velocity field in conjunction with the application of a time-varying driving force.

In some embodiments, Joule heating using an electric field is used to provide a temperature gradient. In some embodiments, the electric field used to provide Joule heating to provide a temperature gradient is the same as the electric field that provides the driving field. In some embodiments, the magnitude of the electric field applied is selected to produce a desired temperature gradient within an affinity matrix.

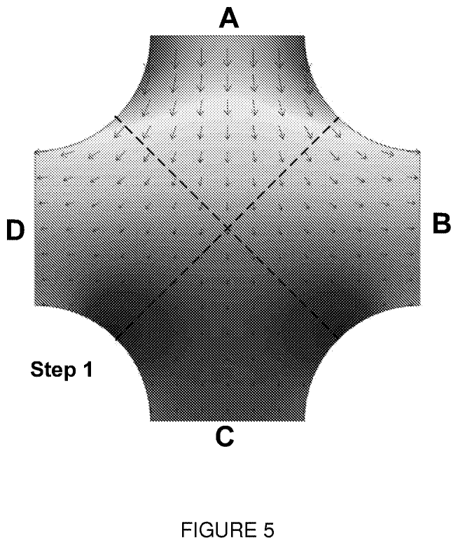

In some embodiments, a spatial temperature gradient is generated using a quadrupole electric field to provide the Joule heating. In some such embodiments, a two dimensional gel with four electrodes is provided. Voltages are applied to the four electrodes such that the electric field in the gel is non-uniform, containing regions of high electric field (and consequently high temperature) and low electric field. The electric field is oriented such that the regions of high electric field tend to push negatively charged molecules towards the center of the gel, while regions of low electric field tend to push such molecules away from the center of the gel. In some such embodiments, the electric field that provides the temperature gradient through Joule heating is also the electric field that applies a driving force to molecules in the gel. An example of such a field pattern is illustrated in FIG. 5. Voltages applied at electrodes A, B, C and D in FIG. 5 are −V, 0, 0, and 0 respectively. Arrows represent the velocity of a negatively charged analyte molecule. Color intensity represents electric field strength. The regions near electrode A have a high electric field strength, which decreases towards electrode C. The high field regions near electrode A tend to push negatively charged molecules towards the center of the gel, while the lower field regions near electrodes B, C, and D tend to push negatively charged molecules away from the center of the gel. In embodiments in which the electric field also provides the temperature gradient, the affinity matrix will become hotter in regions of higher field strength due to Joule heating. Hence, regions of high electric field strength will coincide with regions of higher temperature and thus higher mobility. Accordingly, molecules in the high electric field regions near electrode A will tend to move a greater distance toward the center of the gel, while molecules in the lower electric field regions near electrodes B, C, and D have a lower mobility (are at a cooler temperature) and will move only a short distance away from the center of the gel.

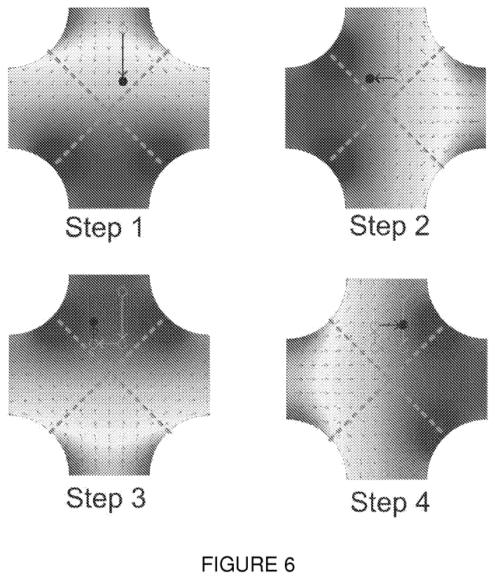

In some embodiments, the electric field pattern of FIG. 5 is rotated in a stepwise manner by rotating the voltage pattern around the four electrodes such that the time averaged electric field is zero as shown in FIG. 6. This rotating field will result in net migration towards the center of the gel for any molecule that is negatively charged and has a mobility that varies with temperature. In some embodiments, the electric field pattern is varied in a manner other than rotation, e.g. by sequentially shifting the voltage pattern by 180°, 90°, 180°, and 90°, or by randomly switching the direction of the electric field. As shown above, the mobility of a molecule moving through an affinity matrix depends on temperature, not electric field strength. The applied electric field will tend to increase the temperature of the matrix through Joule heating; the magnitude of the temperature rise at any given point in the matrix will be proportional to the square of the magnitude of the electric field.

In embodiments in which the thermal gradient is provided by Joule heating produced by the electric field that also provides the driving field, the oscillations in the thermal gradient will have the same period as the electric field oscillations. These oscillations can drive affinity SCODA based concentration in a two dimensional gel.

FIG. 6 illustrates the stepwise rotation of the electric field leading to focusing of molecules whose mobility increases with temperature or electric field according to such an embodiment. A particle path for a negatively charged molecule is shown. After four steps the particle has a net displacement toward the center of the gel. Molecules that do not experience a change in mobility with changing temperature or electric field will experience zero net motion in a zero time averaged electric field.

### Theoretical Predictions of Focusing and Separation

In some embodiments, the electric field and subsequently the Joule heating within an affinity SCODA gel are controlled by both the voltage applied to the source electrodes, and the shape of the gel. Marziali et al. used superimposed rotating dipole and quadrupole fields to drive electrophoretic SCODA concentration. The ratio of the strength of these two fields, the dipole to quadrupole ratio (D/Q), has an impact on the efficiency of SCODA focusing with a maximum at around D/Q=4.5, however the optimum is relatively flat with the SCODA force staying relatively constant for values between 1.75 and 10¹³. One convenient choice of D/Q is 2. With this particular choice, only two distinct potentials need to be applied to the source electrodes, which can be achieved by connecting one electrode to a common voltage rail, grounding the other three, and rotating this pattern in a stepwise manner through the four possible configurations as shown in Table 1. Although analog amplifiers can be used and were used in the examples described herein, using a D/Q ratio of 2 allows one to use discrete MOSFET switches, which simplifies and reduces the required size and complexity of the power supplies.

TABLE 1
Voltage pattern for SCODA focusing with D/Q = 2

| | Electrode A | Electrode B | Electrode C | Electrode D |
|---|---|---|---|---|
| Step 1 | −V | 0 | 0 | 0 |
| Step 2 | 0 | −V | 0 | 0 |
| Step 3 | 0 | 0 | −V | 0 |
| Step 4 | 0 | 0 | 0 | −V |

A starting point for a sequence specific gel geometry was the four-sided gel geometry used for the initial demonstration of electrophoretic SCODA. This geometry can be defined by two numbers, the gel width and the corner radius. The inventors started by using a geometry that had a width of 10 mm and a corner radius of 3 mm. An electro-thermal model of this geometry was implemented in COMSOL Multiphysics® modeling software (COMSOL, Inc., Burlington Mass., USA) to estimate the electric field and temperature profiles within the gel and establish whether or not those field and temperature profiles could drive concentration of a target with a temperature dependent mobility. The model used simultaneously solves Ohm's Law and the heat equation within the domain, using the power density calculated from the solution of Ohm's Law as the source term for the heat equation and using the temperature solution from the heat equation to determine the temperature dependent electrical conductivity of the electrolyte in the gel.

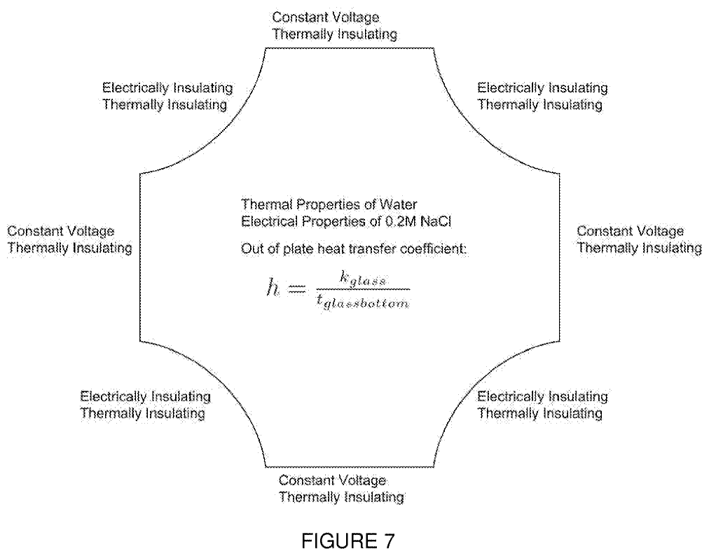

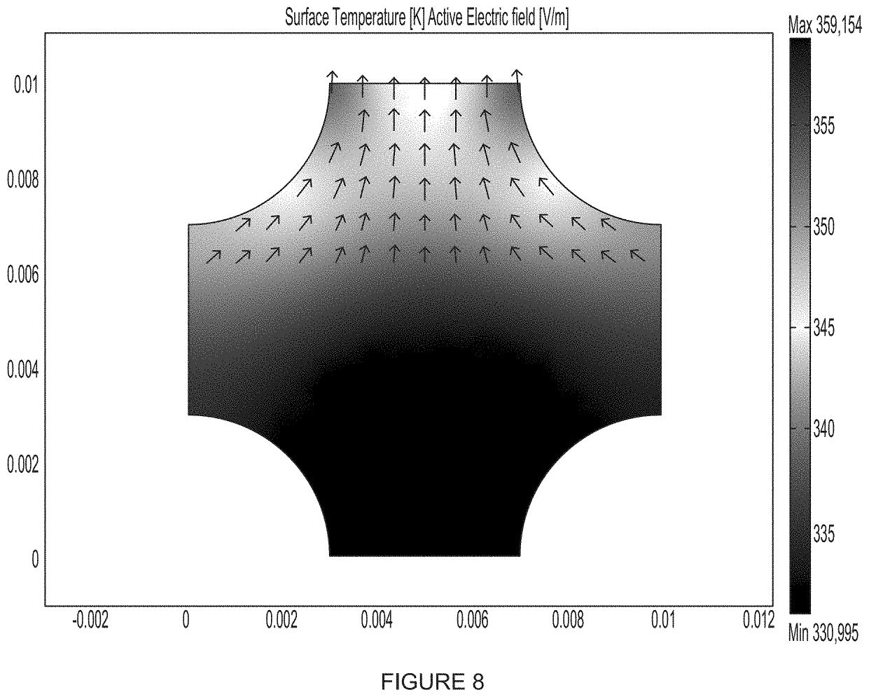

To obtain an accurate estimate of the temperature profile within the gel, the heat conducted out of the top and bottom of the gel are modeled. Boundary conditions and other model parameters are illustrated in FIG. 7. The thermal properties of water and electrical properties of 0.2 M NaCl were used. The gel cassettes are placed on an aluminum spreader plate that acts as a constant temperature reservoir. To model heat flow into the spreader plate the heat transfer coefficient of the glass bottom, given by lilt, was used. The temperature and electric field profiles solved by this model for a single step of the SCODA cycle are shown in FIG. 8. The voltage applied to the four electrodes was −120 V, 0 V, 0 V, 0 V, and the spreader plate temperature was set to 55° C. (328 K). The color map indicates gel temperature and the vector field shows the relative magnitude and direction of the electric field within the gel. Note that as DNA is negatively charged its migration direction will be opposite to the direction of the electric field.

Using experimentally determined values of mobility versus temperature for a given molecule and the thermal model described above, it is possible to determine the SCODA velocity everywhere in the gel for that particular molecule by taking the time average of the instantaneous drift velocity integrated over one complete cycle:

$${\vec{v}}_{s} = \frac{1}{τ} {\int }_{0}^{τ} μ ( T ( \vec{r} , t ) ) \vec{E} ( \vec{r} , t ) dt \qquad [ 33 ]$$

where μ is the temperature dependent mobility, E the electric field and τ the period of the SCODA cycle. The temperature and electric field were solved for four steps in the SCODA cycle and coupled with the mobility function in equation [23]. In this manner, the SCODA velocity everywhere in the gel can be calculated. Since discrete steps are being used, if it is assumed that the period is long enough that the phase lag between the electric field and temperature can be neglected, then the integral in equation [33] becomes a sum:

$${\vec{v}}_{s} = \frac{\sum μ ( {T}_{i} ( \vec{r} ) ) {\vec{E}}_{i} ( \vec{r} ) {t}_{i}}{\sum {t}_{i}} \qquad [ 34 ]$$

where the velocity is summed over all four steps in the cycle.

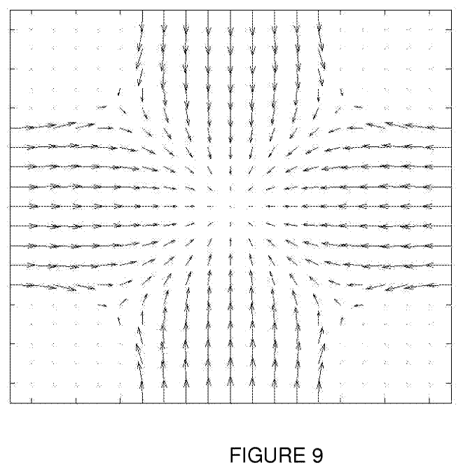

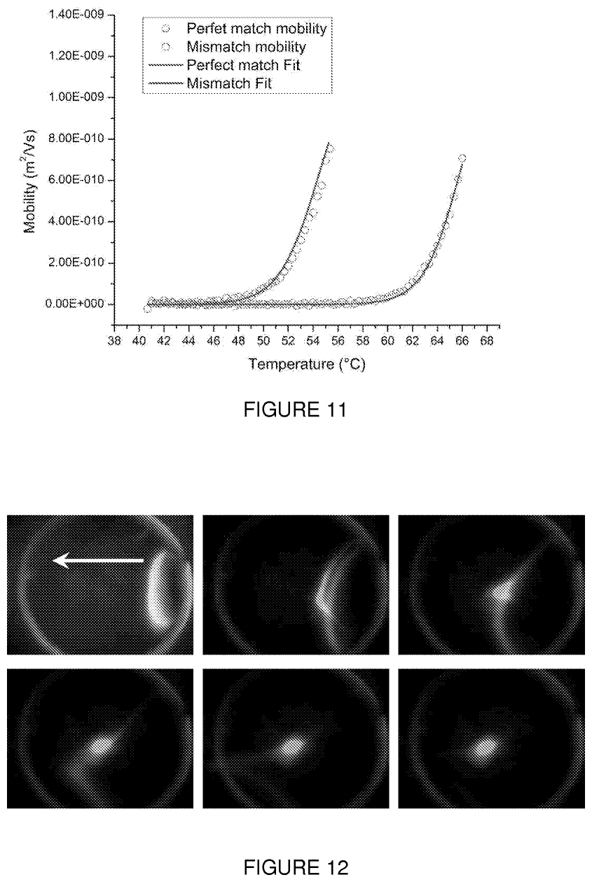

As an example, FIG. 9 shows a vector plot of the SCODA velocity using the experimentally determined mobility versus temperature curve for the perfect match target shown in FIG. 11 (example described below) and the temperature and electric field values calculated above.

The velocity field plotted in FIG. 9 shows a zero velocity point at the geometric center of the gel, with the velocity at all other points in the gel pointing towards the center. Thus, target molecules can be collected within the gel at the center of the electric field pattern.

In embodiments that are used to separate two similar molecules based on differences in binding affinity for the immobilized probe, a washing force is superimposed over the SCODA focusing fields described above. In some embodiments, the washing force is a DC electric field, described herein as a DC bias. For molecules having affinity to the immobilized probe, the SCODA focusing force applied by the SCODA focusing fields described above will tend to counteract movement of a molecule caused by the washing field, i.e. the SCODA focusing fields will tend to exert a restoring force on the molecules and the molecules will be preferentially focused as compared with molecules having a smaller binding affinity. Molecules that have a smaller binding affinity to the immobilized probe will have a greater mobility through the affinity matrix, and the restoring SCODA force will be weaker. As a result, the focus spot of molecules with a smaller binding affinity will be shifted. In some cases, the restoring SCODA force will be so weak that such molecules with a smaller binding affinity will be washed out of the affinity matrix altogether.

In order to enrich for a specific biomolecule from a population of other similar biomolecules using affinity SCODA, one may operate SCODA focusing electric fields with a superimposed DC bias. The DC bias may move the focused molecules off center, in such a way that the molecules with a lower binding energy to the immobilized binding sites move further off center than the molecules with higher binding energies, thus causing the focus to split into multiple foci. For molecules with similar binding energies, this split may be small while washing under bias. The DC bias may be superimposed directly over the focusing fields, or a DC field may be time multiplexed with the focusing fields.

In one exemplary embodiment used to separate nucleic acids having similar sequences, a DC bias is superimposed over the voltage pattern shown in Table 1, resulting in the voltage pattern shown below in Table 2. In some embodiments, the DC bias is applied alternately with the SCODA focusing fields, i.e. the SCODA focusing fields are applied for a period of time then stopped, and the DC bias is applied for a period of time then stopped.

TABLE 2
Applied voltages for focusing under a DC bias.
Shown are values for a 120 V SCODA focusing
potential superimposed over a 10 V DC bias

| | Electrode A | Electrode B | Electrode C | Electrode D |
|---|---|---|---|---|
| Step 1 | −120 | 5 | 10 | 5 |
| Step 2 | 0 | −115 | 10 | 5 |
| Step 3 | 0 | 5 | −110 | 5 |
| Step 4 | 0 | 5 | 10 | −115 |

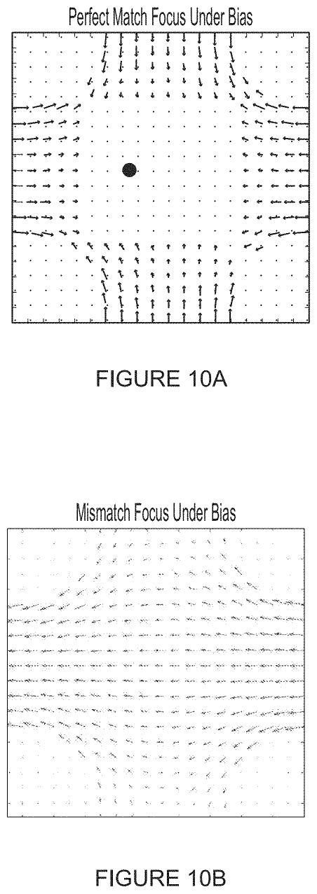

The resulting velocity plots of both the perfect match and single base mismatch targets in the presence of the applied DC bias are shown in FIGS. 10A and 10B, respectively. Electric field and temperature were calculated using COMSOL using a spreader plate temperature of 61° C. Velocity was calculated using equation [34] and the experimentally obtained data fits shown in FIG. 11 (example described below). The zero velocity location of the perfect match target has been moved slightly off center in the direction of the bias (indicated with a circular spot), however the mismatch target has no zero velocity point within the gel. These calculations show that it is possible to completely wash a target with a smaller binding affinity from the immobilized probe from the gel area while capturing the target with a higher binding affinity, enabling selective purification, concentration and/or detection of a specific sequence, even where the nucleotide targets differ in sequence at only one position.

In some embodiments, the optimal combination of the driving field and the mobility altering field used to perform SCODA focusing where there is a maximum difference in focusing force between similar molecules is empirically determined by measuring the velocity of sample molecules through a medium as a function of the mobility varying field. For example, in some embodiments the mobility of a desired target molecule and a non-desired target molecule at various temperatures is measured in an affinity matrix as described above, and the temperature range at which the difference in relative mobility is greatest is selected as the temperature range for conducting affinity SCODA. In some embodiments, the focusing force is proportional to the rate at which the velocity changes with respect to the perturbing field dv/df, where v is the molecule velocity and f the field strength. One skilled in the art may maximize dv/df so as to maximize SCODA focusing and to enable fast washing of contaminants that do not focus. To maximally separate two similar molecules, affinity SCODA may be carried out under conditions such that dvₐ/df−dv_{b}/df (where vₐ is the velocity of molecule a, and v_{b} is the velocity of molecule b) is maximized.

In some embodiments, the strength of the electric field applied to an affinity matrix is calculated so that the highest temperature within the gel corresponds approximately to the temperature at which the difference in binding affinity between two molecules to be separated is highest.

In some embodiments, the temperature at which the difference in binding affinity between the two molecules to be separated is highest corresponds to the temperature at which the difference between the melting temperature of a target molecule and the affinity agent and the melting temperature of a non-target molecule and the affinity agent is highest. In some embodiments, the maximum difference between the melting temperature of a target molecule and the affinity agent and the melting temperature of a non-target molecule and the affinity agent is less than about 9.3° C., in some embodiments less than about 7.8° C., in some embodiments less than about 5.2° C., and in some embodiments less than about 0.7° C.

In some embodiments, the ratio of target molecules to non-target molecules that can be separated by affinity SCODA is any ratio from 1:1 to 1:10,000 and any value there between, e.g. 1:100 or 1:1,000. In some embodiments, after conducting affinity SCODA, the ratio of non-target molecules relative to target molecules that is located in a focus spot of the target molecules has been reduced by a factor of up to 10,000 fold.

### Phase Lag Induced Rotation

In some embodiments, to separate molecules with different affinities for the immobilized affinity agent, a DC bias is superimposed over the SCODA focusing fields as described above. If the separation in binding energy is great enough then the mismatched target can be washed entirely off of the gel. The ability to wash weakly focusing contaminating fragments from the gel can be affected by the phase lag induced rotation discussed above, where the SCODA velocity of a two dimensional system was given by:

\vec{υ}_{SCODA}=|υ_{SCODA}|(cos(ϕ)\hat{r}+sin(ϕ)\hat{θ}) [35]

where ϕ is the phase lag between the electric field oscillations and the mobility varying oscillations. Aside from reducing the proportion of the SCODA velocity that contributes to concentration this result has additional implications when washing weakly focusing contaminants out of an affinity matrix. The rotational component will add to the DC bias and can result in zero or low velocity points in the gel that can significantly increase the time required to wash mismatched targets from the gel.

To counteract the effects of a rotational component of motion that may arise in embodiments in which there is a phase lag between the electric field oscillations and the mobility varying oscillations, the direction in which the SCODA focusing fields are applied may be rotated periodically. In some embodiments, the direction in which the SCODA focusing fields are rotated is altered once every period.

### Optical Feedback

In some embodiments where one molecule of interest (the target molecule) is concentrated in an affinity matrix while a second, similar, molecule (the non-target molecule) is washed off of the affinity matrix, optical feedback may be used to determine when washing is complete and/or to avoid running the target molecule out of the affinity matrix.

The two foci of similar molecules may be close together geographically, and optical feedback may be used to ensure the molecule of interest is not washed off the gel. For example, using a fluorescent surrogate for the molecule of interest or the contaminating molecules (or both) one can monitor their respective positions while focusing under bias, and use that geographical information to adjust the bias ensuring that the molecule of interest is pushed as close to the edge of the gel as possible but not off, while the contaminating molecule may be removed from the gel.

In some embodiments, the molecules to be separated are differentially labeled, e.g. with fluorescent tags of a different color. Real-time monitoring using fluorescence detection can be used to determine when the non-target molecule has been washed off of the affinity matrix, or to determine when the foci of the target molecule and the non-target molecule are sufficiently far apart within the affinity matrix to allow both foci to be separately extracted from the affinity matrix.

In some embodiments, fluorescent surrogate molecules that focus similarly to the target and/or non-target molecules may be used to perform optical feedback. By using a fluorescent surrogate for a target molecule, a non-target molecule, or both a target molecule and a non-target molecule, the respective positions of the target molecule and/or the non-target molecule can be monitored while performing affinity focusing under a washing bias. The location of the surrogate molecules within the affinity matrix can be used to adjust the washing bias to ensure that the molecule of interest is pushed as close to the edge of the gel as possible but not off, while the contaminating molecule may be washed off the gel.

In some embodiments, fluorescent surrogate molecules that focus similarly to the target and/or non-target molecules but will not amplify in any subsequent PCR reactions that may be conducted can be added to a sample to be purified. The presence of the fluorescent surrogate molecules within the affinity matrix enables the use of optical feedback to control SCODA focusing conditions in real time. Fluorescence detection can be used to visualize the position of the fluorescent surrogate molecules in the affinity matrix. In embodiments where the fluorescent surrogate mimics the focusing behavior of the target molecule, the applied washing force can be decreased when the fluorescent surrogate approaches the edge of the affinity matrix, to avoid washing the target molecule out of the affinity matrix. In embodiments where the fluorescent surrogate mimics the focusing behavior of the non-target molecule that is to be separated from the target molecule, the applied washing force can be decreased or stopped after the fluorescent surrogate has been washed out of the affinity matrix, or alternatively when the location of the fluorescent surrogate approaches the edge of the affinity matrix.

### Separation of Differentially Modified Molecules

In some embodiments, molecules that are identical except for the presence or absence of a chemical modification that alters the binding affinity of the molecule for a probe are separated using affinity SCODA. Some embodiments of affinity SCODA are sufficiently sensitive to separate two molecules that have only a small difference in binding affinity for the immobilized affinity agent. Examples of such molecules include differentially modified molecules, such as methylated and unmethylated nucleic acids, methylated or acetylated proteins, or the like.

For example, it has been previously shown that methylation of cytosine residues increases the binding energy of hybridization relative to unmethylated DNA sequences. RNA sequences would be expected to display a similar increase in the binding energy of hybridization when methylated as compared with unmethylated sequences. The inventors have shown that one embodiment of affinity SCODA can be used to separate nucleic acid sequences differing only by the presence of a single methylated cytosine residue. Other chemical modifications would be expected to alter the binding energy of a nucleic acid and its complimentary sequence in a similar manner. Modification of proteins, such as through methylation, can also alter the binding affinity of a protein of interest with a protein, RNA or DNA aptamer, antibody, or other molecule that binds to the protein at or near the methylation site. Accordingly, embodiments of affinity SCODA can be used to separate differentially modified molecules of interest. While the examples herein are directed to methylation enrichment, affinity SCODA can also be applied to enrichment and selection of molecules with other chemical differences, including e.g. acetylation.

Affinity SCODA, and sequence-specific SCODA, may be used to enrich a specific sequence of methylated DNA out of a background of methylated and unmethylated DNA. In this application of affinity SCODA, the strength of the SCODA focusing force may be related to the binding energy of the target DNA to the bound oligonucleotides. Target molecules with a higher binding energy may be made to focus more strongly than targets with lower binding energy. Methylation of DNA has previously been documented to slightly increase the binding energy of target DNA to its complementary sequence. Small changes in binding energy of a complementary oligonucleotide may be exploited through affinity SCODA to preferentially enrich for methylated DNA. SCODA operating conditions may be chosen, for example as described above, such that the methylated DNA is concentrated while unmethylated DNA of the same sequence is washed off the gel.

Some embodiments can separate molecules with a difference in binding energy to an immobilized affinity agent of less than kT, the thermal excitation energy of the target molecules. Some embodiments can separate molecules with a difference in binding energy to an immobilized affinity agent of less than 0.19 kcal/mol. Some embodiments can separate molecules with a difference in binding energy to an immobilized affinity agent of less than 2.6 kcal/mol. Some embodiments can separate molecules with a difference in binding energy to an immobilized affinity agent of less than 3.8 kcal/mol. Some embodiments can separate molecules that differ only by the presence of a methyl group. Some embodiments can separate nucleic acid sequences that differ in sequence at only one base.

### Applications of Affinity SCODA

Systems and methods for separating, purifying, concentrating and/or detecting differentially modified molecules as described above can be applied in fields where detection of biomarkers, specific nucleotide sequences or differentially modified molecules is important, e.g. epigenetics, fetal DNA detection, pathogen detection, cancer screening and monitoring, detection of organ failure, detection of various disease states, and the like. For example, in some embodiments affinity SCODA is used to separate, purify, concentrate and/or detect differentially methylated DNA in such fields as fetal diagnostic tests utilizing maternal body fluids, pathogen detection in body fluids, and biomarker detection in body fluids for detecting cancer, organ failure, or other disease states and for monitoring the progression or treatment of such conditions.

In some embodiments, a sample of bodily fluid or a tissue sample is obtained from a subject. Cells may be lysed, genomic DNA is sheared, and the sample is subjected to affinity SCODA. In some embodiments, molecules concentrated using affinity SCODA are subjected to further analysis, e.g. DNA sequencing, digital PCR, fluorescence detection, or the like, to assay for the presence of a particular biomarker or nucleotide sequence. In some embodiments, the subject is a human.

It is known that fetal DNA is present in maternal plasma, and that differential methylation of maternal versus fetal DNA obtained from the maternal plasma can be used to screen for genetic disorders (see e.g. Poon et al., 2002, Clinical Chemistry 48:1, 35-41). However, one problem that is difficult to overcome is discrimination between fetal and maternal DNA. Affinity SCODA as described above may be used to preferentially separate, purify, concentrate and/or detect DNA which is differentially methylated in fetal DNA versus maternal DNA. For example, affinity SCODA may be used to concentrate or detect DNA which is methylated in the fetal DNA, but not in maternal DNA, or which is methylated in maternal DNA but not fetal DNA. In some embodiments, a sample of maternal plasma is obtained from a subject and subjected to affinity SCODA using an oligonucleotide probe directed to a sequence of interest. The detection of two foci after the application of SCODA focusing fields may indicate the presence of DNA which is differentially methylated as between the subject and the fetus. Comparison to a reference sample from a subject that exhibits a particular genetic disorder may be used to determine if the fetus may be at risk of having the genetic disorder. Further analysis of the sample of DNA obtained through differential modification SCODA through conventional methods such as PCR, DNA sequencing, digital PCR, fluorescence detection, or the like, may be used to assess the risk that the fetus may have a genetic disorder.

One embodiment of the present systems and methods is used to detect abnormalities in fetal DNA, including chromosome copy number abnormalities. Regions of different chromosomes that are known to be differentially methylated in fetal DNA as opposed to maternal DNA are concentrated using affinity SCODA to separate fetal DNA from maternal DNA based on the differential methylation of the fetal DNA in a maternal plasma sample. Further analysis of the separated fetal DNA is conducted (for example using qPCR, DNA sequencing, fluorescent detection, or other suitable method) to count the number of copies from each chromosome and determine copy number abnormalities.

Most cancers are a result of a combination of genetic changes and epigenetic changes, such as changes in DNA methylation (e.g. hypomethylation and/or hypermethylation of certain regions, see e.g. Ehrich, 2002, Oncogene 21:35, 5400-5413). Affinity SCODA can be used to separate, purify, concentrate and/or detect DNA sequences of interest to screen for oncogenes which are abnormally methylated. Embodiments of affinity SCODA are used in the detection of biomarkers involving DNA having a different methylation pattern in cancerous or pre-cancerous cells than in healthy cells. Detection of such biomarkers may be useful in both early cancer screening, and in the monitoring of cancer development or treatment progress. In some embodiments, a sample obtained from a subject, e.g. a sample of a bodily fluid such as plasma or a biopsy, may be processed and analyzed by differential modification SCODA using oligonucleotide probes directed to a sequence of interest. The presence of two foci during the application of SCODA fields may indicate the presence of differential methylation at the DNA sequence of interest. Comparison of the sample obtained from the subject with a reference sample (e.g. a sample from a healthy patient and/or a sample known to originate from cancerous or pre-cancerous tissue) can indicate whether the cells of the subject are at risk of being cancerous or pre-cancerous. Further analysis of the sample of DNA obtained through differential modification SCODA through conventional methods such as PCR, DNA sequencing, digital PCR, fluorescence detection, or the like, may be used to assess the risk that the sample includes cells that may be cancerous or pre-cancerous, to assess the progression of a cancer, or to assess the effectiveness of treatment.

In some embodiments, a specific nucleotide sequence is captured in the gel regardless of methylation (i.e. without selecting for a particular methylation status of the nucleic acid). Undesired nucleotide sequences and/or other contaminants may be washed off the gel while the specific nucleotide sequence remains bound by oligonucleotide probes immobilized within the separation medium. Then, differential methylation SCODA is used to focus the methylated version of the sequence while electrically washing the unmethylated sequence toward a buffer chamber or another gel where it can then be recovered. In some embodiments, the unmethylated sequence could be preferentially extracted.

In some embodiments, biomolecules in blood related to disease states or infection are selectively concentrated using affinity SCODA. In some embodiments, the biomolecules are unique nucleic acids with sequence or chemical differences that render them useful biomarkers of disease states or infection. Following such concentration, the biomarkers can be detected using PCR, sequencing, or similar means. In some embodiments, a sample of bodily fluid or tissue is obtained from a subject, cells are lysed, genomic DNA is sheared, and affinity SCODA is performed using oligonucleotide probes that are complimentary to a sequence of interest. Affinity SCODA is used to detect the presence of differentially methylated populations of the nucleic acid sequence of interest. The presence of differentially methylated populations of the target sequence of interest may indicate a likelihood that the subject suffers from a particular disease state or an infection.

In some embodiments, the focusing pattern of the target nucleic acid produced by affinity SCODA from a subject is compared with the focusing pattern of the target nucleic acid produced by affinity SCODA from one or more reference samples (e.g. an equivalent sample obtained from a healthy subject, and/or an equivalent sample obtained from a subject known to be suffering from a particular disease). Similarities between the focusing pattern produced by the sample obtained from the subject and a reference sample obtained from a subject known to be suffering from a particular disease indicate a likelihood that the subject is suffering from the same disease. Differences between the focusing pattern produced from the sample obtained from the subject and a reference sample obtained from a healthy subject indicate a likelihood that the subject may be suffering from a disease. Differences in the focusing pattern produced from the sample obtained from the subject and a reference sample obtained from a healthy subject may indicate the presence of a differential modification or a mutation in the subject as compared with the healthy subject.

### Use of Multiple Affinity Agents to Capture Multiple Target Molecules

In some embodiments, affinity SCODA is used to separate, purify, concentrate and/or detect more than one sequence per sample. The examples described herein demonstrate that it is possible to concentrate target DNA at probe concentrations as low as 1 μM, as well as with probe concentrations as high as 100 μM. In some embodiments, multiplexed concentration is be performed by immobilizing a plurality of different affinity agents in the medium to provide an affinity matrix. In some embodiments, at least two different affinity agents are immobilized within a medium to separate, purify, concentrate and/or detect at least two different target molecules. In some embodiments, each one of the affinity agents is an oligonucleotide probe with a different sequence. In some embodiments, anywhere between 2 and 100 different oligonucleotide probes are immobilized within a medium to provide an affinity matrix, and anywhere between 2 and 100 different target molecules are separated, purified, concentrated and/or detect simultaneously in a single affinity gel. Each one of the target molecules may be labeled with a different tag to facilitate detection, for example each one of the target molecules could be labeled with a different color of fluorescent tag.

In some embodiments where the binding energy between each of the two or more affinity agents and the two or more target molecules differs, the two or more target molecules may be differentially separated within the affinity matrix by the application of SCODA focusing fields at an appropriate temperature. In some embodiments, a first target molecule with a lower melting temperature for its corresponding affinity agent may be preferentially separated from a second target molecule with a relatively higher melting temperature for its corresponding affinity agent. In some such embodiments, the first molecule is preferentially concentrated by conducting SCODA focusing at a temperature that is sufficiently low that a second target molecule with a relatively higher melting temperature for its corresponding affinity agent does not focus efficiently (i.e. a temperature at which the mobility of the second target molecule within the affinity matrix is relatively low), but sufficiently high to enable efficient focusing of the first molecule. In some such embodiments, the first and second molecules are differentially separated through the application of a washing bias, e.g. a DC bias, at a temperature that is sufficiently low that the second target molecule is not displaced or is displaced only slowly by the washing bias, but sufficiently high that the first target molecule is displaced or is displaced at a higher velocity by the washing bias.

### Apparatus for Performing Affinity SCODA

In some embodiments, affinity SCODA is performed on an electrophoresis apparatus comprising a region for containing the affinity matrix, buffer reservoirs, power supplies capable of delivering large enough voltages and currents to cause the desired effect, precise temperature control of the SCODA medium (which is a gel in some embodiments), and a two color fluorescence imaging system for the monitoring of two different molecules in the SCODA medium.

### Sample Preparation Process

In some aspects, the disclosure provides processes for preparing a sample, e.g., for detection and/or analysis. In some embodiments, a process described herein may be used to identify properties or characteristics of a sample, including the identity or sequence (e.g., nucleotide sequence or amino acid sequence) of one or more target molecules in the sample. In some embodiments, a process may include one or more sample transformation steps, such as sample lysis, sample purification, sample fragmentation, purification of a fragmented sample, library preparation (e.g., nucleic acid library preparation), purification of a library preparation, sample enrichment (e.g., using affinity SCODA), and/or detection/analysis of a target molecule.

In some embodiments, a sample may be a purified sample, a cell lysate, a single-cell, a population of cells, or a tissue. In some embodiments, a sample is any biological sample. In some embodiments, a sample (e.g., a biological sample) is a blood, saliva, sputum, feces, urine or buccal swab sample. In some embodiments, a biological sample is from a human, a non-human primate, a rodent, a dog, a cat, a horse, or any other mammal. In some embodiments, a biological sample is from a bacterial cell culture (e.g., an E. coli bacterial cell culture). A bacterial cell culture may comprise gram positive bacterial cells and/or gram negative bacterial cells. In some embodiments, a sample is a purified sample of nucleic acids or proteins that have been previously extracted via user-developed methods from metagenomic samples or environmental samples. A blood sample may be a freshly drawn blood sample from a subject (e.g., a human subject) or a dried blood sample (e.g., preserved on solid media (e.g. Guthrie cards)). A blood sample may comprise whole blood, serum, plasma, red blood cells, and/or white blood cells.

In some embodiments, a sample (e.g., a sample comprising cells or tissue), may be lysed (e.g., disrupted, degraded and/or otherwise digested) in a process in accordance with the instant disclosure. In some embodiments, a sample comprising cells or tissue is lysed using any one of known physical or chemical methodologies to release a target molecule (e.g., a target nucleic acid or a target protein) from said cells or tissues. In some embodiments, a sample may be lysed using an electrolytic method, an enzymatic method, a detergent-based method, and/or mechanical homogenization. In some embodiments, a sample (e.g., complex tissues, gram positive or gram negative bacteria) may require multiple lysis methods performed in series. In some embodiments, if a sample does not comprise cells or tissue (e.g., a sample comprising purified nucleic acids), a lysis step may be omitted. In some embodiments, lysis of a sample is performed to isolate target nucleic acid(s). In some embodiments, lysis of a sample is performed to isolate target protein(s). In some embodiments, a lysis method further includes use of a mill to grind a sample, sonication, surface acoustic waves (SAW), freeze-thaw cycles, heating, addition of detergents, addition of protein degradants (e.g., enzymes such as hydrolases or proteases), and/or addition of cell wall digesting enzymes (e.g., lysozyme or zymolase). Exemplary detergents (e.g., non-ionic detergents) for lysis include polyoxyethylene fatty alcohol ethers, polyoxyethylene alkylphenyl ethers, polyoxyethylene-polyoxypropylene block copolymers, polysorbates and alkylphenol ethoxylates, preferably nonylphenol ethoxylates, alkylglucosides and/or polyoxyethylene alkyl phenyl ethers. In some embodiments, lysis methods involve heating a sample for at least 1-30 min, 1-25 min, 5-25 min, 5-20 min, 10-30 min, 5-10 min, 10-20 min, or at least 5 min at a desired temperature (e.g., at least 60° C., at least 70° C., at least 80° C., at least 90° C., or at least 95° C.).

In some embodiments, a sample (e.g., a sample comprising a target nucleic acid or a target protein) may be purified, e.g., following lysis, in a process in accordance with the instant disclosure. In some embodiments, a sample may be purified using chromatography (e.g., affinity chromatography that selectively binds the sample) or electrophoresis. In some embodiments, a sample may be purified in the presence of precipitating agents. In some embodiments, after a purification step or method, a sample may be washed and/or released from a purification matrix (e.g., affinity chromatography matrix) using an elution buffer. In some embodiments, a purification step or method may comprise the use of a reversibly switchable polymer, such as an electroactive polymer. In some embodiments, a sample may be purified by electrophoretic passage of a sample through a porous matrix (e.g., cellulose acetate, agarose, acrylamide).

In some embodiments, a sample (e.g., a sample comprising a target nucleic acid or a target protein) may be fragmented in a process in accordance with the instant disclosure. In some embodiments, a nucleic acid sample may be fragmented to produce small (<1 kilobase) fragments for sequence specific identification to large (up to 10+ kilobases) fragments for long read sequencing applications. Fragmentation of nucleic acids or proteins may, in some embodiments, be accomplished using mechanical (e.g., fluidic shearing), chemical (e.g., iron (Fe+) cleavage) and/or enzymatic (e.g., restriction enzymes, tagmentation using transposases) methods. In some embodiments, a protein sample may be fragmented to produce peptide fragments of any length. Fragmentation of proteins may, in some embodiments, be accomplished using chemical and/or enzymatic (e.g., proteolytic enzymes such as trypsin) methods. In some embodiments, mean fragment length may be controlled by reaction time, temperature, and concentration of sample and/or enzymes (e.g., restriction enzymes, transposases). In some embodiments, a nucleic acid may be fragmented by tagmentation such that the nucleic acid is simultaneously fragmented and labeled with a fluorescent molecule (e.g., a fluorophore). In some embodiments, a fragmented sample may be subjected to a round of purification (e.g., chromatography or electrophoresis) to remove small and/or undesired fragments as well as residual payload, chemicals and/or enzymes (e.g., transposases) used during the fragmentation step. For example, a fragmented sample (e.g., sample comprising nucleic acids) may be purified from an enzyme (e.g., a transposase), wherein the purification comprises denaturing the enzyme (e.g., by a combination of heat, chemical (e.g. SDS), and enzymatic (e.g. proteinase K) processes).

In some embodiments, a sample comprising a target nucleic acid may be used to generate a nucleic acid library for subsequent analysis (e.g., genomic sequencing) in a process in accordance with the instant disclosure. A nucleic acid library may be a linear library or a circular library. In some embodiments, nucleic acids of a circular library may comprise elements that allow for downstream linearization (e.g., endonuclease restriction sites, incorporation of uracil). In some embodiments, a nucleic acid library may be purified (e.g., using chromatography, e.g., affinity chromatography), or electrophoresis.

In some embodiments, a library of nucleic acids (e.g., linear nucleic acids) is prepared using end-repair, a process wherein a combination of enzymes (e.g., Taq DNA Ligase, Endonuclease IV, Bst DNA Polymerase, Fpg, Uracil-DNA Glycosylase, T4 Endonuclease V and/or Endonuclease VIII) extend the 3′ end of the nucleic acids, generating a complement to the 5′ payload, and repairing any abasic sites or nicks in the nucleic acids. In some embodiments, a library of linear nucleic acids is prepared using a self-priming hairpin adaptor, a process which may obviate the need to anneal a unique sequencing primer to an individual nucleic acid fragment primer prior to formation of a polymerase complex. Following end-repair, a library of nucleic acids (e.g., linear nucleic acids) may be purified using solid-phase adsorption with subsequent elution into a fresh buffer, using passage of the nucleic acids through a size-selective matrix (e.g., agarose gel). The size-selective matrix may be used to remove nucleic acid fragments that are smaller than the size of the target nucleic acids.

In some embodiments, a sample (e.g., a sample comprising a target nucleic acid or a target protein) may be enriched for a target molecule in a process in accordance with the instant disclosure. In some embodiments, a sample is enriched for a target molecule using an electropheretic method. In some embodiments, a sample is enriched for a target molecule using affinity SCODA. In some embodiments, a sample is enriched for a target molecule using field inversion gel electrophoresis (FIGE). In some embodiments, a sample is enriched for a target molecule using pulsed field gel electrophoresis (PFGE). In some embodiments, the matrix used during enrichment (e.g., a porous media, electrophoretic polymer gel) comprises immobilized affinity agents (also known as ‘immobilized capture probes’) that bind to target molecule present in the sample. In some embodiments, a matrix used during enrichment comprises 1, 2, 3, 4, 5, or more unique immobilized capture probes, each of which binds to a unique target molecule and/or bind to the same target molecule with different binding affinities.

In some embodiments, an immobilized capture probe is an oligonucleotide capture probe that hybridizes to a target nucleic acid. In some embodiments, an oligonucleotide capture probe is at least 50%, 60%, 70%, 80%, 90% 95%, or 100% complementary to a target nucleic acid. In some embodiments, a single oligonucleotide capture probe may be used to enrich a plurality of related target nucleic acids (e.g., 2, 3, 4, 5, 6, 7, 8, 9, 10, 20, 30, 40, 50, or more related target nucleic acids) that share at least 50%, 60%, 70%, 80%, 90% 95%, or 99% sequence identity. Enrichment of a plurality of related target nucleic acids may allow for the generation of a metagenomic library. In some embodiments, an oligonucleotide capture probe may enable differential enrichment of related target nucleic acids. In some embodiments, an oligonucleotide capture probe may enable enrichment of a target nucleic acid relative to a nucleic acid of identical sequence that differs in its modification state (e.g., single nucleotide polymorphism, methylation state, acetylation state). In some embodiments, an oligonucleotide capture probe is used to enrich human genomic DNA for a specific gene of interest (e.g., HLA). A specific gene of interest may be a gene that is relevant to a specific disease state or disorder. In some embodiments, an oligonucleotide capture probe is used to enrich nucleic acid(s) of a metagenomic sample.

In some embodiments, for the purposes of enriching nucleic acid target molecules with a length of 0.5-2 kilobases, oligonucleotide capture probes may be covalently immobilized in an acrylamide matrix using a 5′ Acrydite moiety. In some embodiments, for the purposes of enriching larger nucleic acid target molecules (e.g., with a length of >2 kilobases), oligonucleotide capture probes may be immobilized in an agarose matrix. In some embodiments, oligonucleotide capture probes may be immobilized in an agarose matrix using thiol-epoxide chemistries (e.g., by covalently attached thiol-modified oligonucleotides to crosslinked agarose beads). Oligonucleotide capture probes linked to agarose beads can be combined and solidified within standard agarose matrices (e.g., at the same agarose percentage).

In some embodiments, enrichment of nucleic acids using methods described herein (e.g., enrichment using SCODA) produces nucleic acid target molecules that comprise a length of about 0.5 kilobases (kb), about 1 kb, about 1.5 kb, about 2 kb, about 3 kb, about 4 kb, about 5 kb, about 6 kb, about 7 kb, about 8 kb, about 9 kb, about 10 kb, about 12 kb, about 15 kb, about 20 kb, or more. In some embodiments, enrichment of nucleic acids using methods described herein (e.g., enrichment using SCODA) produces nucleic acid target molecules that comprise a length of about 0.5-2 kb, 0.5-5 kb, 1-2 kb, 1-3 kb, 1-4 kb, 1-5 kb, 1-10 kb, 2-10 kb, 2-5 kb, 5-10 kb, 5-15 kb, 5-20 kb, 5-25 kb, 10-15 kb, 10-20 kb, or 10-25 kb.

In some embodiments, an immobilized capture probe is a protein capture probe (e.g., an aptamer or an antibody) that binds to a target protein or peptide fragment. In some embodiments, a protein capture probe binds to a target protein or peptide fragment with a binding affinity of 10⁻⁹ to 10⁻⁸ M, 10⁻⁸ to 10⁻⁷ M, 10⁻⁷ to 10⁻⁶ M, 10⁻⁶ to 10⁻⁵ M, 10⁻⁵ to 10⁻⁴ M, 10⁻⁴ to 10⁻³ M, or 10⁻³ to 10⁻² M. In some embodiments, the binding affinity is in the picomolar to nanomolar range (e.g., between about 10⁻¹² and about 10⁻⁹ M). In some embodiments, the binding affinity is in the nanomolar to micromolar range (e.g., between about 10⁻⁹ and about 10⁻⁶ M). In some embodiments, the binding affinity is in the micromolar to millimolar range (e.g., between about 10⁻⁶ and about 10⁻³ M). In some embodiments, the binding affinity is in the picomolar to micromolar range (e.g., between about 10⁻¹² and about 10⁻⁶ M). In some embodiments, the binding affinity is in the nanomolar to millimolar range (e.g., between about 10⁻⁹ and about 10⁻³ M). In some embodiments, a single protein capture probe may be used to enrich a plurality of related target proteins that share at least 50%, 60%, 70%, 80%, 90% 95%, or 99% sequence identity. In some embodiments, a single protein capture probe may be used to enrich a plurality of related target proteins (e.g., 2, 3, 4, 5, 6, 7, 8, 9, 10, 20, 30, 40, 50, or more related target proteins) that share at least 50%, 60%, 70%, 80%, 90% 95%, or 99% sequence homology. Enrichment of a plurality of related target proteins may allow for the generation of a metaproteomics library. In some embodiments, a protein capture probe may enable differential enrichment of related target proteins.

In some embodiments, multiple capture probes (e.g., populations of multiple capture probe types, e.g., that bind to deterministic target molecules of infectious agents such as adenovirus, staphylococcus, pneumonia, or tuberculosis) may be immobilized in an enrichment matrix. Application of a sample to an enrichment matrix with multiple deterministic capture probes may result in diagnosis of a disease or condition (e.g., presence of an infectious agent).

In some embodiments, a target molecule or related target molecules may be released from the enrichment matrix after removal of non-target molecules, in a process in accordance with the instant disclosure. In some embodiments, a target molecule may be released from the enrichment matrix by increasing the temperature of the enrichment matrix. Adjusting the temperature of the matrix further influences migration rate as increased temperatures provide a higher capture probe stringency, requiring greater binding affinities between the target molecule and the capture probe. In some embodiments, when enriching related target molecules, the matrix temperature may be gradually increased in a step-wise manner in order to release and isolate target molecules in steps of ever-increasing homology. In some embodiments, temperature is increased by about 5%, 10%, 15%, 20%, 25%, 30%, 40%, 50%, or more in each step or over a period of time (e.g., 1-10 min, 1-5 min, or 4-8 min). In some embodiments, temperature is increased by 5%-10%, 5-15%, 5%-20%, 5%-25%, 5%-30%, 5%-40%, 5%-50%, 10%-25%, 20%-30%, 30%-40%, 35%-50%, or 40%-70% in each step or over a period of time (e.g., 1-10 min, 1-5 min, or 4-8 min). In some embodiments, temperature is increased by about 1° C., 2° C., 3° C., 4° C., 5° C., 6° C., 7° C., 8° C., 9° C., or 10° C. in each step or over a period of time (e.g., 1-10 min, 1-5 min, or 4-8 min). In some embodiments, temperature is increased by 1-10° C., 1-5° C., 2-5° C., 2-10° C., 3-8° C., 4-9° C., or 5-10° C. in each step or over a period of time (e.g., 1-10 min, 1-5 min, or 4-8 min). This may allow for the sequencing of target proteins or target nucleic acids that are increasingly distant in their relation to an initial reference target molecule, enabling discovery of novel proteins (e.g., enzymes) or functions (e.g., enzymatic function or gene function). In some embodiments, when using multiple capture probes (e.g., multiple deterministic capture probes), the matrix temperature may be increased in a step-wise or gradient fashion, permitting temperature-dependent release of different target molecules and resulting in generation of a series of barcoded release bands that represent the presence or absence of control and target molecules.

Enrichment of a sample (e.g., a sample comprising a target nucleic acid or a target protein) allows for a reduction in the total volume of the sample. For example, in some embodiments, the total volume of a sample is reduced after enrichment by at least 10%, at least 20%, at least 30%, at least 40%, at least 50%, at least 60%, at least 70%, at least 80%, at least 90%, at least 100%, or at least 120%. In some embodiments, the total volume of a sample is reduced after enrichment from 1-20 mL initial volume to 100-1000 μL final volume, from 1-5 mL initial volume to 100-1000 μL final volume, from 100-1000 μL initial volume to 25-100 μL final volume, from 100-500 μL initial volume to 10-100 μL final volume, or from 50-200 μL initial volume to 1-25 μL final volume. For example, in some embodiments, the final volume of a sample after enrichment is 10-100 μL, 10-50 μL, 10-25 μL, 20-100 μL, 20-50 μL, 25-100 μL, 25-250 μL, 25-1000 μL, 100-1000 μL, 100-500 μL, 100-250 μL, 200-1000 μL, 200-500 μL, 200-750 μL, 500-1000 μL, 500-1500 μL, 500-750 μL, 1-5 mL, 1-10 mL, 1-2 mL, 1-3 mL, or 1-4 mL.

In some embodiments, a target molecule or target molecules may be detected after enrichment and subsequent release to enable analysis of said target molecule(s) and its upstream sample, in a process in accordance with the instant disclosure. In some embodiments, a target nucleic acid may be detected using gene sequencing, absorbance, fluorescence, electrical conductivity, capacitance, surface plasmon resonance, hybrid capture, antibodies, direct labeling of the nucleic acid (e.g., end-labeling, labeled tagmentation payloads), non-specific labeling with intercalating dyes (e.g., ethidium bromide, SYBR dyes), or any other known methodology for nucleic acid detection. In some embodiments, a target protein or peptide fragment may be detected using absorbance, fluorescence, mass spectroscopy, amino acid sequencing, or any other known methodology for protein or peptide detection.

### Sample Preparation Devices and Modules

Devices or modules including apparatuses, cartridges (e.g., comprising channels (e.g., microfluidic channels)), and/or pumps (e.g., peristaltic pumps) for use in a process of preparing a sample for analysis are generally provided. Devices can be used in accordance with the instant disclosure to enable capture, concentration, manipulation, and/or detection of a target molecule from a biological sample. In some embodiments, devices and related methods are provided for automated processing of a sample to produce material for next generation sequencing and/or other downstream analytical techniques. Devices and related methods may be used for performing chemical and/or biological reactions, including reactions for nucleic acid and/or protein processing in accordance with sample preparation or sample analysis processes described elsewhere herein.

In some embodiments, a sample preparation device or module is positioned to deliver or transfer to a sequencing module or device a target molecule or a plurality of target molecules (e.g., target nucleic acids or target proteins). In some embodiments, a sample preparation device or module is connected directly to (e.g., physically attached to) or indirectly to a sequencing device or module.

In some embodiments, a sample preparation device or module is used to prepare a sample for diagnostic purposes. In some embodiments, a sample preparation device that is used to prepare a sample for diagnostic purposes is positioned to deliver or transfer to a diagnostic module or diagnostic device a target molecule or a plurality of molecules (e.g., target nucleic acids or target proteins). In some embodiments, a sample preparation device or module is connected directly to (e.g., physically attached to) or indirectly to a diagnostic device.

In some embodiments, a device comprises a cartridge housing that is configured to receive one or more cartridges (e.g., configured to receive one cartridge at a time). In some embodiments, a cartridge comprises one or more reservoirs or reaction vessels configured to receive a fluid and/or contain one or more reagents used in a sample preparation process. In some embodiments, a cartridge comprises one or more channels (e.g., microfluidic channels) configured to contain and/or transport a fluid (e.g., a fluid comprising one or more reagents) used in a sample preparation process. Reagents include buffers, enzymatic reagents, polymer matrices, capture reagents, size-specific selection reagents, sequence-specific selection reagents, and/or purification reagents. Additional reagents for use in a sample preparation process are described elsewhere herein.

In some embodiments, a cartridge includes one or more stored reagents (e.g., of a liquid or lyophilized form suitable for reconstitution to a liquid form). The stored reagents of a cartridge include reagents suitable for carrying out a desired process and/or reagents suitable for processing a desired sample type. In some embodiments, a cartridge is a single-use cartridge (e.g., a disposable cartridge) or a multiple-use cartridge (e.g., a reusable cartridge). In some embodiments, a cartridge is configured to receive a user-supplied sample. The user-supplied sample may be added to the cartridge before or after the cartridge is received by the device, e.g., manually by the user or in an automated process. In some embodiments, a cartridge is a sample preparation cartridge. In some embodiments, a sample preparation cartridge is capable of isolating or purifying a target molecule (e.g., a target nucleic acid or target protein) from a sample (e.g., a biological sample).

In some embodiments, a cartridge comprises an affinity matrix for enrichment as described herein. In some embodiments, a cartridge comprises an affinity matrix for enrichment using affinity SCODA, FIGE, or PFGE. In some embodiments, a cartridge comprises an affinity matrix comprising an immobilized affinity agent that has a binding affinity for a target nucleic acid or target protein.

In some embodiments, a sample preparation device of the disclosure produces (e.g., enriches or purifies) target nucleic acids with an average read-length for downstream sequencing applications that is longer than an average read-length produced using control methods (e.g., Sage BluePippin methods, manual methods (e.g., manual bead-based size selection methods)). In some embodiments, a sample preparation device produces target nucleic acids with an average read-length for sequencing that comprises at least 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 nucleotides in length. In some embodiments, a sample preparation device produces target nucleic acids with an average read-length for sequencing that comprises 700-3000, 1000-3000, 1000-2500, 1000-2400, 1000-2300, 1000-2200, 1000-2100, 1000-2000, 1000-1900, 1000-1800, 1000-1700, 1000-1600, 1000-1500, 1000-1400, 1000-1300, 1000-1200, 1500-3000, 1500-2500, 1500-2000, or 2000-3000 nucleotides in length.

Devices in accordance with the instant disclosure generally contain mechanical and electronic and/or optical components which can be used to operate a cartridge as described herein. In some embodiments, the device components operate to achieve and maintain specific temperatures on a cartridge or on specific regions of the cartridge. In some embodiments, the device components operate to apply specific voltages for specific time durations to electrodes of a cartridge. In some embodiments, the device components operate to move liquids to, from, or between reservoirs and/or reaction vessels of a cartridge. In some embodiments, the device components operate to move liquids through channel(s) of a cartridge, e.g., to, from, or between reservoirs and/or reaction vessels of a cartridge. In some embodiments, the device components move liquids via a peristaltic pumping mechanism (e.g., apparatus) that interacts with an elastomeric, reagent-specific reservoir or reaction vessel of a cartridge. In some embodiments, the device components move liquids via a peristaltic pumping mechanism (e.g., apparatus) that is configured to interact with an elastomeric component (e.g., surface layer comprising an elastomer) associated with a channel of a cartridge to pump fluid through the channel. Device components can include computer resources, for example, to drive a user interface where sample information can be entered, specific processes can be selected, and run results can be reported.

In some embodiments, a cartridge is capable of handling small-volume fluids (e.g., 1-10 μL, 2-10 μL, 4-10 μL, 5-10 μL, 1-8 μL, or 1-6 μL fluid). In some embodiments, the sequencing cartridge is physically embedded or associated with a sample preparation device or module (e.g., to allow for a prepared sample to be delivered to a reaction mixture for sequencing. In some embodiments, a sequencing cartridge that is physically embedded or associated with a sample preparation device or module comprises microfluidic channels that have fluid interfaces in the form of face sealing gaskets or conical press fits (e.g., Luer fittings). In some embodiments, fluid interfaces can then be broken after delivery of the prepared sample in order to physically separate the sequencing cartridge from the sample preparation device or module.

The following non-limiting example is meant to illustrate aspects of the devices, methods, and compositions described herein. The use of a sample preparation device or module in accordance with the instant disclosure may proceed with one or more of the following described steps. A user may open the lid of the device and insert a cartridge that supports the desired process. The user may then add a sample, which may be combined with a specific lysis solution, to a sample port on the cartridge. The user may then close the device lid, enter any sample specific information via a touch screen interface on the device, select any process specific parameters (e.g., range of desired size selection, desired degree of homology for target molecule capture, etc.), and initiate the sample preparation process run. Following the run, the user may receive relevant run data (e.g., confirmation of successful completion of the run, run specific metrics, etc.), as well as process specific information (e.g., amount of sample generated, presence or absence of specific target sequence, etc.). Data generated by the run may be subjected to subsequent bioinformatics analysis, which can be either local or cloud based. Depending on the process, a finished sample may be extracted from the cartridge for subsequent use (e.g., genomic sequencing, qPCR quantification, cloning, etc.). The device may then be opened, and the cartridge may then be removed.

In some embodiments, the sample preparation module comprises a pump. In some embodiments, the pump is peristaltic pump. Some such pumps comprise one or more of the inventive components for fluid handling described herein. For example, the pump may comprise an apparatus and/or a cartridge. In some embodiments, the apparatus of the pump comprises a roller, a crank, and a rocker. In some such embodiments, the crank and the rocker are configured as a crank-and-rocker mechanism that is connected to the roller. The coupling of a crank-and-rocker mechanism with the roller of an apparatus can, in some cases, allow for certain of the advantages describe herein to be achieved (e.g., facile disengagement of the apparatus from the cartridge, well-metered stroke volumes). In certain embodiments, the cartridge of the pump comprises channels (e.g., microfluidic channels). In some embodiments, at least a portion of the channels of the cartridge have certain cross-sectional shapes and/or surface layers that may contribute to any of a number of advantages described herein.

One non-limiting aspect of some cartridges that may, in some cases, provide certain benefits is the inclusion of channels having certain cross-sectional shapes in the cartridges. For example, in some embodiments, the cartridge comprises v-shaped channels. One potentially convenient but non-limiting way to form such v-shaped channels is by molding or machining v-shaped grooves into the cartridge. The recognized advantages of including a v-shaped channel (also referred to herein as a v-groove or a channel having a substantially triangularly-shaped cross-section) in certain embodiments in which a roller of the apparatus engages with the cartridge to cause fluid flow through the channels. For example, in some instances, a v-shaped channel is dimensionally insensitive to the roller. In other words, in some instances, there is no single dimension to which the roller (e.g., a wedge shaped roller) of the apparatus must adhere in order to suitably engage with the v-shaped channel. In contrast, certain conventional cross sectional shapes of the channels, such as semi-circular, may require that the roller have a certain dimension (e.g., radius) in order to suitably engage with the channel (e.g., to create a fluidic seal to cause a pressure differential in a peristaltic pumping process). In some embodiments, the inclusion of channels that are dimensionally insensitive to rollers can result in simpler and less expensive fabrication of hardware components and increased configurability/flexibility.

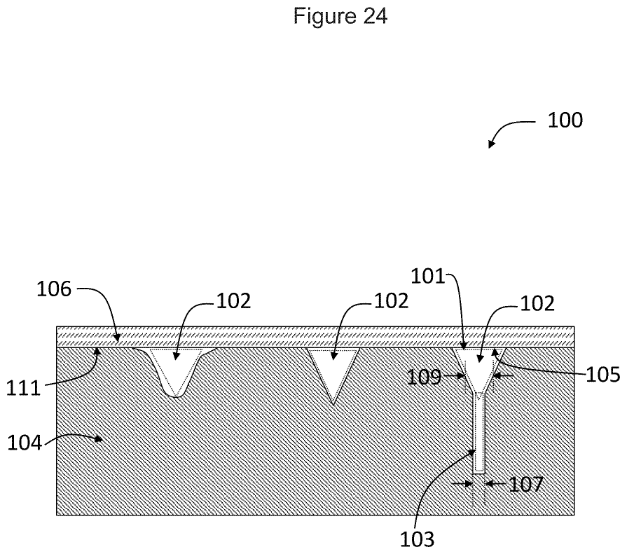

In certain aspects, the cartridges comprise a surface layer (e.g., a flat surface layer). One exemplary aspect relates to potentially advantageous embodiments involving layering a membrane (also referred to herein as a surface layer) comprising (e.g., consisting essentially of) an elastomer (e.g., silicone) above the v-groove, to produce, in effect, half of a flexible tube. FIG. 24 depicts an exemplary cartridge 100 according to certain such embodiments, and is described in more detail below. Then, in some embodiments, by deforming the surface layer comprising an elastomer into the channel to form a pinch and by then translating the pinch, negative pressure can be generated on the trailing edge of the pinch which creates suction and positive pressure can be generated on the leading edge of the pinch, pumping fluid in the direction of the leading edge of the pinch. In certain embodiments, this pumping by interfacing a cartridge (comprising channels having a surface layer) with an apparatus comprising a roller, which apparatus is configured to carry out a motion of the roller that includes engaging the roller with a portion of the surface layer to pinch the portion of the surface layer with the walls and/or base of the associated channel, translating the roller along the walls and/or base of the associated channel in a rolling motion to translate the pinch of the surface layer against the walls and/or base, and/or disengaging the roller with a second portion of the surface layer. In certain embodiments, a crank-and-rocker mechanism is incorporated into the apparatus to carry out this motion of the roller.

A conventional peristaltic pump generally involves tubing having been inserted into an apparatus comprising rollers on a rotating carriage, such that the tubing is always engaged with the remainder of the apparatus as the pump functions. By contrast, in certain embodiments, channels in cartridges herein are linear or comprise at least one linear portion, such that the roller engages with a horizontal surface. In certain embodiments, the roller is connected to a small roller arm that is spring-loaded so that the roller can track the horizontal surface while continuously pinching a portion of the surface layer. Spring loading the apparatus (e.g., a roller arm of the apparatus) can in some cases help regulate the force applied by the apparatus (e.g., roller) to the surface layer and a channel of a cartridge.

In certain embodiments, each rotation of the crank in a crank-and-rocker mechanism connected to the roller provides a discrete pumping volume. In certain embodiments, it is straightforward to park the apparatus in a disengaged position, where the roller is disengaged from any cartridge. In certain embodiments, forward and backward pumping motions are fairly symmetrical as provided by apparatuses described herein, such that a similar amount of force (torque) (e.g., within 10%) is required for forward and backward pumping motions.

In certain embodiments, it may be advantageous to, for a particular size of apparatus, have a relatively high crank radius (e.g., greater than or equal to 2 mm, optionally including associated linkages). Consequently, it may, in certain embodiments, also be advantageous to have a relatively high stroke length (e.g., greater than or equal to 10 mm) to engage with an associated cartridge. Having relatively high crank radius and stroke length, in certain embodiments, ensures no mechanical interference between the apparatus and the cartridge when moving components of the apparatus relative to the cartridge.

In certain embodiments, having v-shaped grooves advantageously allows for utilization with rollers of a variety of sizes having a wedge-shaped edge. By contrast, for example, having a rectangular channel rather than a v-groove results in the width of the roller associated with the rectangular channel needing to be more controlled and precise in relation to the width of the rectangular channel, and results in the forces being applied to the rectangular channel needing to be more precise. Similarly, the channel(s) having a semicircular cross-section may also require more controlled and precise dimension for the width of the associated roller.

In certain embodiments, an apparatus described herein may comprise a multi-axis system (e.g., robot) configured so as to move at least a portion of the apparatus in a plurality of dimensions (e.g., two dimensions, three dimensions). For example, the multi-axis system may be configured so as to move at least a portion of the apparatus to any pumping lane location among associated cartridge(s). For example, in certain embodiments, a carriage herein may be functionally connected to a multi-axis system. In certain embodiments, a roller may be indirectly functionally connected to a multi-axis system. In certain embodiments, an apparatus portion, comprising a crank-and-rocker mechanism connected to a roller, may be functionally connected to a multi-axis system. In certain embodiments, each pumping lane may be addressed by location and accessed by an apparatus described herein using a multi-axis system.

### Nucleic Acid Sequencing Process

Some aspects of the instant disclosure further involve sequencing nucleic acids (e.g., deoxyribonucleic acids or ribonucleic acid). In some aspects, compositions, devices, systems, and techniques described herein can be used to identify a series of nucleotides incorporated into a nucleic acid (e.g., by detecting a time-course of incorporation of a series of labeled nucleotides). In some embodiments, compositions, devices, systems, and techniques described herein can be used to identify a series of nucleotides that are incorporated into a template-dependent nucleic acid sequencing reaction product synthesized by a polymerizing enzyme (e.g., RNA polymerase).

Accordingly, also provided herein are methods of determining the sequence of a target nucleic acid. In some embodiments, the target nucleic acid is enriched (e.g., enriched using electrophoretic methods, e.g., affinity SCODA) prior to determining the sequence of the target nucleic acid. In some embodiments, provided herein are methods of determining the sequences of a plurality of target nucleic acids (e.g., at least 2, 3, 4, 5, 10, 15, 20, 30, 50, or more) present in a sample (e.g., a purified sample, a cell lysate, a single-cell, a population of cells, or a tissue). In some embodiments, a sample is prepared as described herein (e.g., lysed, purified, fragmented, and/or enriched for a target nucleic acid) prior to determining the sequence of a target nucleic acid or a plurality of target nucleic acids present in a sample. In some embodiments, a target nucleic acid is an enriched target nucleic acid (e.g., enriched using electrophoretic methods, e.g., affinity SCODA).

In some embodiments, methods of sequencing comprise steps of: (i) exposing a complex in a target volume to one or more labeled nucleotides, the complex comprising a target nucleic acid or a plurality of nucleic acids present in a sample, at least one primer, and a polymerizing enzyme; (ii) directing one or more excitation energies, or a series of pulses of one or more excitation energies, towards a vicinity of the target volume; (iii) detecting a plurality of emitted photons from the one or more labeled nucleotides during sequential incorporation into a nucleic acid comprising one of the at least one primers; and (iv) identifying the sequence of incorporated nucleotides by determining one or more characteristics of the emitted photons.

In another aspect, the instant disclosure provides methods of sequencing target nucleic acids or a plurality of target nucleic acids present in a sample by sequencing a plurality of nucleic acid fragments, wherein the target nucleic acid(s) comprises the fragments. In certain embodiments, the method comprises combining a plurality of fragment sequences to provide a sequence or partial sequence for the parent nucleic acid (e.g., parent target nucleic acid). In some embodiments, the step of combining is performed by computer hardware and software. The methods described herein may allow for a set of related nucleic acids (e.g., two or more nucleic acids present in a sample), such as an entire chromosome or genome to be sequenced.

In some embodiments, a primer is a sequencing primer. In some embodiments, a sequencing primer can be annealed to a nucleic acid (e.g., a target nucleic acid) that may or may not be immobilized to a solid support. A solid support can comprise, for example, a sample well (e.g., a nanoaperture, a reaction chamber) on a chip or cartridge used for nucleic acid sequencing. In some embodiments, a sequencing primer may be immobilized to a solid support and hybridization of the nucleic acid (e.g., the target nucleic acid) further immobilizes the nucleic acid molecule to the solid support. In some embodiments, a polymerase (e.g., RNA Polymerase) is immobilized to a solid support and soluble sequencing primer and nucleic acid are contacted to the polymerase. In some embodiments a complex comprising a polymerase, a nucleic acid (e.g., a target nucleic acid) and a primer is formed in solution and the complex is immobilized to a solid support (e.g., via immobilization of the polymerase, primer, and/or target nucleic acid). In some embodiments, none of the components are immobilized to a solid support. For example, in some embodiments, a complex comprising a polymerase, a target nucleic acid, and a sequencing primer is formed in situ and the complex is not immobilized to a solid support.

In some embodiments, sequencing by synthesis methods can include the presence of a population of target nucleic acid molecules (e.g., copies of a target nucleic acid) and/or a step of amplification (e.g., polymerase chain reaction (PCR)) of a target nucleic acid to achieve a population of target nucleic acids. However, in some embodiments, sequencing by synthesis is used to determine the sequence of a single nucleic acid molecule in any one reaction that is being evaluated and nucleic acid amplification may not be required to prepare the target nucleic acid. In some embodiments, a plurality of single molecule sequencing reactions are performed in parallel (e.g., on a single chip or cartridge) according to aspects of the instant disclosure. For example, in some embodiments, a plurality of single molecule sequencing reactions are each performed in separate sample wells (e.g., nanoapertures, reaction chambers) on a single chip or cartridge.

In some embodiments, sequencing of a target nucleic acid molecule comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) nucleotides of the target nucleic acid. In some embodiments, the at least two nucleotides are contiguous nucleotides. In some embodiments, the at least two amino acids are non-contiguous nucleotides.

In some embodiments, sequencing of a target nucleic acid comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all nucleotides in the target nucleic acid. For example, in some embodiments, sequencing of a target nucleic acid comprises identification of less than 100% of one type of nucleotide in the target nucleic acid. In some embodiments, sequencing of a target nucleic acid comprises identification of less than 100% of each type of nucleotide in the target nucleic acid.

### Protein Sequencing Process

Aspects of the instant disclosure also involve methods of protein sequencing and identification, methods of polypeptide sequencing and identification, methods of amino acid identification, and compositions, systems, and devices for performing such methods. Such protein sequencing and identification is performed, in some embodiments, with the same instrument that performs sample preparation and/or genome sequencing, described in more detail herein. In some aspects, methods of determining the sequence of a target protein are described. In some embodiments, the target protein is enriched (e.g., enriched using electrophoretic methods, e.g., affinity SCODA) prior to determining the sequence of the target protein. In some aspects, methods of determining the sequences of a plurality of proteins (e.g., at least 2, 3, 4, 5, 10, 15, 20, 30, 50, or more) present in a sample (e.g., a purified sample, a cell lysate, a single-cell, a population of cells, or a tissue) are described. In some embodiments, a sample is prepared as described herein (e.g., lysed, purified, fragmented, and/or enriched for a target protein) prior to determining the sequence of a target protein or a plurality of proteins present in a sample. In some embodiments, a target protein is an enriched target protein (e.g., enriched using electrophoretic methods, e.g., affinity SCODA)

In some embodiments, the instant disclosure provides methods of sequencing and/or identifying an individual protein in a sample comprising a plurality of proteins by identifying one or more types of amino acids of a protein from the mixture. In some embodiments, one or more amino acids (e.g., terminal amino acids or internal amino acids) of the protein are labeled (e.g., directly or indirectly, for example using a binding agent) and the relative positions of the labeled amino acids in the protein are determined. In some embodiments, the relative positions of amino acids in a protein are determined using a series of amino acid labeling and cleavage steps. In some embodiments, the relative position of labeled amino acids in a protein can be determined without removing amino acids from the protein but by translocating a labeled protein through a pore (e.g., a protein channel) and detecting a signal (e.g., a Förster resonance energy transfer (FRET) signal) from the labeled amino acid(s) during translocation through the pore in order to determine the relative position of the labeled amino acids in the protein molecule.

In some embodiments, the identity of a terminal amino acid (e.g., an N-terminal or a C-terminal amino acid) is determined prior to the terminal amino acid being removed and the identity of the next amino acid at the terminal end being assessed; this process may be repeated until a plurality of successive amino acids in the protein are assessed. In some embodiments, assessing the identity of an amino acid comprises determining the type of amino acid that is present. In some embodiments, determining the type of amino acid comprises determining the actual amino acid identity (e.g., determining which of the naturally-occurring 20 amino acids an amino acid is, e.g., using a binding agent that is specific for an individual terminal amino acid). However, in some embodiments, assessing the identity of a terminal amino acid type can comprise determining a subset of potential amino acids that can be present at the terminus of the protein. In some embodiments, this can be accomplished by determining that an amino acid is not one or more specific amino acids (i.e., and therefore could be any of the other amino acids). In some embodiments, this can be accomplished by determining which of a specified subset of amino acids (e.g., based on size, charge, hydrophobicity, binding properties) could be at the terminus of the protein (e.g., using a binding agent that binds to a specified subset of two or more terminal amino acids).

In some embodiments, a protein or polypeptide can be digested into a plurality of smaller proteins or polypeptides and sequence information can be obtained from one or more of these smaller proteins or polypeptides (e.g., using a method that involves sequentially assessing a terminal amino acid of a protein and removing that amino acid to expose the next amino acid at the terminus).

In some embodiments, a protein is sequenced from its amino (N) terminus. In some embodiments, a protein is sequenced from its carboxy (C) terminus. In some embodiments, a first terminus (e.g., N or C terminus) of a protein is immobilized and the other terminus (e.g., the C or N terminus) is sequenced as described herein.

As used herein, sequencing a protein refers to determining sequence information for a protein. In some embodiments, this can involve determining the identity of each sequential amino acid for a portion (or all) of the protein. In some embodiments, this can involve determining the identity of a fragment (e.g., a fragment of a target protein or a fragment of a sample comprising a plurality of proteins). In some embodiments, this can involve assessing the identity of a subset of amino acids within the protein (e.g., and determining the relative position of one or more amino acid types without determining the identity of each amino acid in the protein). In some embodiments amino acid content information can be obtained from a protein without directly determining the relative position of different types of amino acids in the protein. The amino acid content alone may be used to infer the identity of the protein that is present (e.g., by comparing the amino acid content to a database of protein information and determining which protein(s) have the same amino acid content).

In some embodiments, sequence information for a plurality of protein fragments obtained from a target protein or sample comprising a plurality of proteins (e.g., via enzymatic and/or chemical cleavage) can be analyzed to reconstruct or infer the sequence of the target protein or plurality of proteins present in the sample. Accordingly, in some embodiments, the one or more types of amino acids are identified by detecting luminescence of one or more labeled affinity reagents that selectively bind the one or more types of amino acids. In some embodiments, the one or more types of amino acids are identified by detecting luminescence of a labeled protein.

In some embodiments, the instant disclosure provides compositions, devices, and methods for sequencing a protein by identifying a series of amino acids that are present at a terminus of a protein over time (e.g., by iterative detection and cleavage of amino acids at the terminus). In yet other embodiments, the instant disclosure provides compositions, devices, and methods for sequencing a protein by identifying labeled amino content of the protein and comparing to a reference sequence database.

In some embodiments, the instant disclosure provides compositions, devices, and methods for sequencing a protein by sequencing a plurality of fragments of the protein. In some embodiments, sequencing a protein comprises combining sequence information for a plurality of protein fragments to identify and/or determine a sequence for the protein. In some embodiments, combining sequence information may be performed by computer hardware and software. The methods described herein may allow for a set of related proteins, such as an entire proteome of an organism, to be sequenced. In some embodiments, a plurality of single molecule sequencing reactions are performed in parallel (e.g., on a single chip or cartridge) according to aspects of the instant disclosure. For example, in some embodiments, a plurality of single molecule sequencing reactions are each performed in separate sample wells on a single chip or cartridge.

In some embodiments, methods provided herein may be used for the sequencing and identification of an individual protein in a sample comprising a plurality of proteins. In some embodiments, the instant disclosure provides methods of uniquely identifying an individual protein in a sample comprising a plurality of proteins. In some embodiments, an individual protein is detected in a mixed sample by determining a partial amino acid sequence of the protein. In some embodiments, the partial amino acid sequence of the protein is within a contiguous stretch of approximately 5-50, 10-50, 25-50, 25-100, or 50-100 amino acids.

Without wishing to be bound by any particular theory, it is expected that most human proteins can be identified using incomplete sequence information with reference to proteomic databases. For example, simple modeling of the human proteome has shown that approximately 98% of proteins can be uniquely identified by detecting just four types of amino acids within a stretch of 6 to 40 amino acids (see, e.g., Swaminathan, et al. PLoS Comput Biol. 2015, 11(2):e1004080; and Yao, et al. Phys. Biol. 2015, 12(5):055003). Therefore, a sample comprising a plurality of proteins can be fragmented (e.g., chemically degraded, enzymatically degraded) into short protein fragments of approximately 6 to 40 amino acids, and sequencing of this protein-based library would reveal the identity and abundance of each of the proteins present in the original sample. Compositions and methods for selective amino acid labeling and identifying polypeptides by determining partial sequence information are described in in detail in U.S. patent application Ser. No. 15/510,962, filed Sep. 15, 2015, entitled “SINGLE MOLECULE PEPTIDE SEQUENCING,” which is incorporated herein by reference in its entirety.

Sequencing in accordance with the instant disclosure, in some aspects, may involve immobilizing a protein (e.g., a target protein) on a surface of a substrate (e.g., of a solid support, for example a chip or cartridge, for example in a sequencing device or module as described herein). In some embodiments, a protein may be immobilized on a surface of a sample well (e.g., on a bottom surface of a sample well) on a substrate. In some embodiments, the N-terminal amino acid of the protein is immobilized (e.g., attached to the surface). In some embodiments, the C-terminal amino acid of the protein is immobilized (e.g., attached to the surface). In some embodiments, one or more non-terminal amino acids are immobilized (e.g., attached to the surface). The immobilized amino acid(s) can be attached using any suitable covalent or non-covalent linkage, for example as described in this disclosure. In some embodiments, a plurality of proteins are attached to a plurality of sample wells (e.g., with one protein attached to a surface, for example a bottom surface, of each sample well), for example in an array of sample wells on a substrate.

In some embodiments, the identity of a terminal amino acid (e.g., an N-terminal or a C-terminal amino acid) is determined, then the terminal amino acid is removed, and the identity of the next amino acid at the terminal end is determined. This process may be repeated until a plurality of successive amino acids in the protein are determined. In some embodiments, determining the identity of an amino acid comprises determining the type of amino acid that is present. In some embodiments, determining the type of amino acid comprises determining the actual amino acid identity, for example by determining which of the naturally-occurring 20 amino acids is the terminal amino acid is (e.g., using a binding agent that is specific for an individual terminal amino acid). In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine. In some embodiments, determining the identity of a terminal amino acid type can comprise determining a subset of potential amino acids that can be present at the terminus of the protein. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be at the terminus of the protein (e.g., using a binding agent that binds to a specified subset of two or more terminal amino acids).

In some embodiments, assessing the identity of a terminal amino acid type comprises determining that an amino acid comprises a post-translational modification. Non-limiting examples of post-translational modifications include acetylation, ADP-ribosylation, caspase cleavage, citrullination, formylation, N-linked glycosylation, O-linked glycosylation, hydroxylation, methylation, myristoylation, neddylation, nitration, oxidation, palmitoylation, phosphorylation, prenylation, S-nitrosylation, sulfation, sumoylation, and ubiquitination.

In some embodiments, a protein or protein can be digested into a plurality of smaller proteins and sequence information can be obtained from one or more of these smaller proteins (e.g., using a method that involves sequentially assessing a terminal amino acid of a protein and removing that amino acid to expose the next amino acid at the terminus).

In some embodiments, sequencing of a protein molecule comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) amino acids in the protein molecule. In some embodiments, the at least two amino acids are contiguous amino acids. In some embodiments, the at least two amino acids are non-contiguous amino acids.

In some embodiments, sequencing of a protein molecule comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all amino acids in the protein molecule. For example, in some embodiments, sequencing of a protein molecule comprises identification of less than 100% of one type of amino acid in the protein molecule (e.g., identification of a portion of all amino acids of one type in the protein molecule). In some embodiments, sequencing of a protein molecule comprises identification of less than 100% of each type of amino acid in the protein molecule.

In some embodiments, sequencing of a protein molecule comprises identification of at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 or more types of amino acids in the protein.

### Sequencing Device or Module

Sequencing of nucleic acids or proteins in accordance with the instant disclosure, in some aspects, may be performed using a system that permits single molecule analysis. The system may include a sequencing device or module and an instrument configured to interface with the sequencing device or module. The sequencing device or module may include an array of pixels, where individual pixels include a sample well and at least one photodetector. The sample wells of the sequencing device or module may be formed on or through a surface of the sequencing device or module and be configured to receive a sample placed on the surface of the sequencing device or module. In some embodiments, the sample wells are a component of a cartridge (e.g., a disposable or single-use cartridge) that can be inserted into the device. Collectively, the sample wells may be considered as an array of sample wells. The plurality of sample wells may have a suitable size and shape such that at least a portion of the sample wells receive a single target molecule or sample comprising a plurality of molecules (e.g., a target nucleic acid or a target protein). In some embodiments, the number of molecules within a sample well may be distributed among the sample wells of the sequencing device or module such that some sample wells contain one molecule (e.g., a target nucleic acid or a target protein) while others contain zero, two, or a plurality of molecules.

In some embodiments, a sequencing device or module is positioned to receive a target molecule or sample comprising a plurality of molecules (e.g., a target nucleic acid or a target protein) from a sample preparation device or module. In some embodiments, a sequencing device or module is connected directly (e.g., physically attached to) or indirectly to a sample preparation device or module.

Excitation light is provided to the sequencing device or module from one or more light sources external to the sequencing device or module. Optical components of the sequencing device or module may receive the excitation light from the light source and direct the light towards the array of sample wells of the sequencing device or module and illuminate an illumination region within the sample well. In some embodiments, a sample well may have a configuration that allows for the target molecule or sample comprising a plurality of molecules to be retained in proximity to a surface of the sample well, which may ease delivery of excitation light to the sample well and detection of emission light from the target molecule or sample comprising a plurality of molecules. A target molecule or sample comprising a plurality of molecules positioned within the illumination region may emit emission light in response to being illuminated by the excitation light. For example, a nucleic acid or protein (or pluralities thereof) may be labeled with a fluorescent marker, which emits light in response to achieving an excited state through the illumination of excitation light. Emission light emitted by a target molecule or sample comprising a plurality of molecules may then be detected by one or more photodetectors within a pixel corresponding to the sample well with the target molecule or sample comprising a plurality of molecules being analyzed. When performed across the array of sample wells, which may range in number between approximately 10,000 pixels to 1,000,000 pixels according to some embodiments, multiple sample wells can be analyzed in parallel.

The sequencing device or module may include an optical system for receiving excitation light and directing the excitation light among the sample well array. The optical system may include one or more grating couplers configured to couple excitation light to the sequencing device or module and direct the excitation light to other optical components. The optical system may include optical components that direct the excitation light from a grating coupler towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the sequencing device or module by improving the uniformity of excitation light received by sample wells of the sequencing device or module. Examples of suitable components, e.g., for coupling excitation light to a sample well and/or directing emission light to a photodetector, to include in a sequencing device or module are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated herein by reference in their entirety. Examples of suitable grating couplers and waveguides that may be implemented in the sequencing device or module are described in U.S. patent application Ser. No. 15/844,403, filed Dec. 15, 2017, titled “OPTICAL COUPLER AND WAVEGUIDE SYSTEM,” which is incorporated herein by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the sequencing device or module, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” which is incorporated herein by reference in its entirety.

Components located off of the sequencing device or module may be used to position and align an excitation source to the sequencing device or module. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated herein by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference in its entirety. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated herein by reference in its entirety.

The photodetector(s) positioned with individual pixels of the sequencing device or module may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the marker associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the sequencing device or module, and the distribution of arrival times may provide an indication of a timing characteristic of the sample's emission light (e.g., a proxy for luminescence lifetime). In some embodiments, the one or more photodetectors provide an indication of the probability of emission light emitted by the marker (e.g., luminescence intensity). In some embodiments, a plurality of photodetectors may be sized and arranged to capture a spatial distribution of the emission light. Output signals from the one or more photodetectors may then be used to distinguish a marker from among a plurality of markers, where the plurality of markers may be used to identify a sample within the sample. In some embodiments, a sample may be excited by multiple excitation energies, and emission light and/or timing characteristics of the emission light emitted by the sample in response to the multiple excitation energies may distinguish a marker from a plurality of markers.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the sequencing device or module, which may be connected to an instrument interfaced with the sequencing device or module. The electrical signals may be subsequently processed and/or analyzed. Processing and/or analyzing of electrical signals may occur on a suitable computing device either located on or off the instrument.

The instrument may include a user interface for controlling operation of the instrument and/or the sequencing device or module. The user interface may be configured to allow a user to input information into the instrument, such as commands and/or settings used to control the functioning of the instrument. In some embodiments, the user interface may include buttons, switches, dials, and/or a microphone for voice commands. The user interface may allow a user to receive feedback on the performance of the instrument and/or sequencing device or module, such as proper alignment and/or information obtained by readout signals from the photodetectors on the sequencing device or module. In some embodiments, the user interface may provide feedback using a speaker to provide audible feedback. In some embodiments, the user interface may include indicator lights and/or a display screen for providing visual feedback to a user.

In some embodiments, the instrument or device described herein may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the sequencing device or module, and/or data generated from the readout signals of the photodetector.

In some embodiments, the instrument may include a processing device configured to analyze data received from one or more photodetectors of the sequencing device or module and/or transmit control signals to the excitation source(s). In some embodiments, the processing device may comprise a general purpose processor, and/or a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the sequencing device or module.

According to some embodiments, the instrument that is configured to analyze target molecules or samples comprising a plurality of molecules based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region may be less complex to operate and maintain, may be more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference in its entirety. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference in its entirety.

In some embodiments, different numbers of fluorophores of the same type may be linked to different components of a target molecule (e.g., a target nucleic acid or a target protein) or a plurality of molecules present in a sample (e.g., a plurality of nucleic acids or a plurality of proteins), so that each individual molecule may be identified based on luminescence intensity. For example, two fluorophores may be linked to a first labeled molecule and four or more fluorophores may be linked to a second labeled molecule. Because of the different numbers of fluorophores, there may be different excitation and fluorophore emission probabilities associated with the different molecule. For example, there may be more emission events for the second labeled molecule during a signal accumulation interval, so that the apparent intensity of the bins is significantly higher than for the first labeled molecule.

The inventors have recognized and appreciated that distinguishing nucleic acids or proteins based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation. For example, a limited bandwidth of radiation may include a central or peak wavelength within a 20 nm bandwidth output by a pulsed optical source. In some cases, “characteristic wavelength” or “wavelength” may be used to refer to a peak wavelength within a total bandwidth of radiation output by a source.

### Combined Sample Preparation and Sequencing Device

In some embodiments, a device herein comprises a sample preparation module and a sequencing module. In some embodiments, a device that comprises a sample preparation module and a sequencing module involves a sequencing chip or cartridge that is embedded into a sample preparation cartridge, such that the two cartridges comprise a single, inseparable consumable. In some embodiments, the sequencing chip or cartridge requires consumable support electronics (e.g., a PCB substrate with wirebonds, electrical contacts). The consumable support electronics may be in direct physical contact with the sequencing chip or cartridge. In some embodiments, the sequencing chip or cartridge requires an interface for a peristaltic pump, temperature control and/or electrophoresis contacts. These interfaces may allow for precise geometric registration for the many electrical contacts and laser alignment. In some embodiments, different sections of a chip or cartridge may comprise different temperatures, physical forces, electrical interfaces of varying voltage and current, vibration, and/or competing alignment requirements. In some embodiments, disparate instrument sub-systems associated with either the sample preparation or sequencing module must be in close proximity in order to share resources. In some embodiments, a device that comprises a sample preparation module and a sequencing module is hands-free (i.e., can be used without the use of hands).

In some embodiments, a device that comprises a sample preparation module and a sequencing module produces (e.g., enriches or purifies) target nucleic acids with an average read-length for downstream sequencing applications that is longer than an average read-length produced using control methods (e.g., Sage BluePippin methods, manual methods (e.g., manual bead-based size selection methods)). In some embodiments, a sample preparation device produces target nucleic acids with an average read-length for sequencing that comprises at least 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 nucleotides in length. In some embodiments, a sample preparation device produces target nucleic acids with an average read-length for sequencing that comprises 700-3000, 1000-3000, 1000-2500, 1000-2400, 1000-2300, 1000-2200, 1000-2100, 1000-2000, 1000-1900, 1000-1800, 1000-1700, 1000-1600, 1000-1500, 1000-1400, 1000-1300, 1000-1200, 1500-3000, 1500-2500, 1500-2000, or 2000-3000 nucleotides in length.

In some embodiments, a device that comprises a sample preparation module and a sequencing module allows for shortened times between initiation of sample preparation and detection of a target molecule contained within the sample than control or traditional methods (e.g., Sage BluePippin methods followed by sequencing). In some embodiments, a device that comprises a sample preparation module and a sequencing module is capable of detecting a target molecule using sequencing in less time (e.g., 2-fold, 3-fold, 4-fold, 5-fold, or 10-fold less time) than control or traditional methods (e.g., Sage BluePippin methods followed by sequencing).

In some embodiments, a device that comprises a sample preparation module and a sequencing module is capable of detecting a target molecule with lower inputs of sample than control or traditional methods (e.g., Sage BluePippin methods followed by sequencing). In some embodiments, a device of the disclosure requires as little as 0.1 μg, 0.2 μg, 0.3 μg, 0.4 μg, 0.5 μg, 0.6 μg, 0.7 μg, 0.8 μg, 0.9 μg, or 1 μg of sample (e.g., biological sample). In some embodiments, a device of the disclosure requires as little as 10 μL, 20 μL, 30 μL, 40 μL, 50 μL, 60 μL, 70 μL, 80 μL, 90 μL, 100 μL, 110 μL, 130 μL, 150 μL, 175 μL, 200 μL, 225 μL, or 250 μL of sample (e.g., biological sample such as blood).

### Devices or Modules

In some embodiments, devices or modules (e.g., sample preparation devices; sequencing devices; combined sample preparation and sequencing devices) are configured to transport small volume(s) of fluid precisely with a well-defined fluid flow resolution, and with a well-defined flow rate in some cases. In some embodiments, devices or modules are configured to transport fluid at a flow rate of greater than or equal to 0.1 μL/s, greater than or equal to 0.5 μL/s, greater than or equal to 1 μL/s, greater than or equal to 2 μL/s, greater than or equal to 5 μL/s, or higher. In some embodiments, devices or modules herein are configured to transport fluid at a flow rate of less than or equal to 100 μL/s, less than or equal to 75 μL/s, less than or equal to 50 μL/s, less than or equal to 30 μL/s, less than or equal to 20 μL/s, less than or equal to 15 μL/s, or less. Combinations of these ranges are possible. For example, in some embodiments, devices or modules herein are configured to transport fluid at a flow rate of greater than or equal to 0.1 μL/s and less than or equal to 100 μL/s, or greater than or equal to 5 μL/s and less than or equal to 15 μL/s. For example, in certain embodiments, systems, devices, and modules herein have a fluid flow resolution on the order of tens of microliters or hundreds of microliters. Further description of fluid flow resolution is described elsewhere herein. In certain embodiments, systems, devices, and modules are configured to transport small volumes of fluid through at least a portion of a cartridge.

Some aspects relate to configurations of pumps and apparatuses that include a roller (e.g., in combination with a crank-and-rocker mechanism). Other aspects relate to cartridges comprising channels (e.g., microchannels) having cross-sectional shapes (e.g., substantially triangular shapes), valving, deep sections, and/or surface layers (e.g., flat elastomer membranes). Certain aspects relate to a decoupling of certain components of the peristaltic pump (e.g., the roller) from other components of the pump (e.g., pumping lanes). In some cases, certain elements of apparatuses (e.g., edges of the roller) are configured to interact with elements of the cartridge (e.g., surface layers and certain shapes of the channels) in such a way (e.g., via engagement and disengagement) that any of a variety of advantages are achieved. In some non-limiting embodiments, certain inventive features and configurations of the apparatuses, cartridges, and pumps described herein contribute to improved automation of the fluid pumping process (e.g., due to the use of a translatable roller and a separate cartridge containing multiple different fluidic channels that can be indexed by the roller). In some cases, features described herein contribute to an ability to handle a relatively high number of different fluids (e.g., for multiplexing with multiple samples) with a relatively high number of configurations using a relatively small number of hardware components (e.g., due to the use of separate cartridges with multiple different channels, each of which may be accessible to the roller). As one example, in some cases, the features described herein allow for more than one apparatus to be paired with a cartridge to pump more than one lane simultaneously or use two pumps in one lane for other functionality. In some cases, the features contribute to a reduction in required fluid volume and/or less stringent tolerances in roller/channel interactions (e.g., due to inventive cross-sectional shapes of the channels and/or the edge of the roller, and/or due to the use of inventive valving and/or deep sections of channels). In some cases, features described herein result in a reduction in required washing of hardware components (e.g., due to a decoupling of an apparatus and a cartridge of the peristaltic pump). In some embodiments, aspects of the apparatuses, cartridges, and pumps described herein are useful for preparing samples. For example, some such aspects may be incorporated into a sample preparation module upstream of a detection module (e.g., for analysis/sequencing/identification of biologically-derived samples).

In another aspect, peristaltic pumps are provided. In some embodiments, a peristaltic pump comprises a roller and a cartridge, wherein the cartridge comprises a base layer having a surface comprising channels, wherein at least a portion of at least some of the channels (1) have a substantially triangularly-shaped cross-section having a single vertex at a base of the channel and having two other vertices at the surface of the base layer, and (2) have a surface layer, comprising an elastomer, configured to substantially seal off a surface opening of the channel. Embodiments of peristaltic pumps are further described elsewhere herein.

In some embodiments, a system (e.g., pump, device) described herein undergoes a pump cycle. In some embodiments, a pump cycle corresponds to one rotation of a crank of the system. In some embodiments, each pump cycle may transport greater than or equal to 1 μL, greater than or equal to 2 μL, greater than or equal to 4 μL, less than or equal to 10 μL, less than or equal to 8 μL, and/or less than or equal to 6 μL of fluid. Combinations of the above-referenced ranges are also possible (e.g., between or equal to 1 μL and 10 μL). Other ranges of volumes of fluid are also possible.

In some embodiments, a system described herein has a particular stroke length. In certain embodiments, given that each pump cycle may transport on the order of between or equal to 1 μL and 10 μL of fluid, and/or given that channel dimensions may preferably be on the order of 1 mm wide and on the order of 1 mm deep (e.g., depending on what can be machined or molded to decrease channel volume and maintain reasonable tolerances), a stroke length may be greater than or equal to 10 mm, greater than or equal to 12 mm, greater than or equal to 14 mm, less than or equal to 20 mm, less than or equal to 18 mm, and/or less than or equal to 16 mm. Combinations of the above-referenced ranges are also possible (e.g., between or equal to 10 mm and 20 mm). Other ranges are also possible. As used herein, “stroke length” refers to a distance a roller travels while engaged with a substrate. In certain embodiments, the substrate comprises a cartridge.

In another aspect, cartridges are provided. In some embodiments, a cartridge comprises a base layer having a surface comprising channels, and at least a portion of at least some of the channels (1) have a substantially triangularly-shaped cross-section having a single vertex at a base of the channel and having two other vertices at the surface of the base layer, and (2) have a surface layer, comprising an elastomer, configured to substantially seal off a surface opening of the channel. Embodiments of cartridges are further described elsewhere herein.

In some embodiments, a cartridge comprises a base layer. In some embodiments, a base layer has a surface comprising one or more channels. For example, FIG. 24 is a schematic diagram of a cross-section view of a cartridge 100 along the width of channels 102, in accordance with some embodiments. The depicted cartridge 100 includes a base layer 104 having a surface 111 comprising channels 102. In certain embodiments, at least some of the channels are microchannels. For example, in some embodiments, at least some of channels 102 are microchannels. In certain embodiments, all of the channels microchannels. For example, referring again to FIG. 24, in certain embodiments, all of channels 102 are microchannels.

As used herein, the term “channel” will be known to those of ordinary skill in the art and may refer to a structure configured to contain and/or transport a fluid. A channel generally comprises: walls; a base (e.g., a base connected to the walls and/or formed from the walls); and a surface opening that may be open, covered, and/or sealed off at one or more portions of the channel.

As used herein, the term “microchannel” refers to a channel that comprises at least one dimension less than or equal to 1000 microns in size. For example, a microchannel may comprise at least one dimension (e.g., a width, a height) less than or equal to 1000 microns (e.g., less than or equal to 100 microns, less than or equal to 10 microns, less than or equal to 5 microns) in size. In some embodiments, a microchannel comprises at least one dimension greater than or equal to 1 micron (e.g., greater than or equal to 2 microns, greater than or equal to 10 microns). Combinations of the above-referenced ranges are also possible (e.g., greater than or equal to 1 micron and less than or equal to 1000 microns, greater than or equal to 10 micron and less than or equal to 100 microns). Other ranges are also possible. In some embodiments, a microchannel has a hydraulic diameter of less than or equal to 1000 microns. As used herein, the term “hydraulic diameter” (DH) will be known to those of ordinary skill in the art and may be determined as: DH=4 A/P, wherein A is a cross-sectional area of the flow of fluid through the channel and P is a wetted perimeter of the cross-section (a perimeter of the cross-section of the channel contacted by the fluid).

In some embodiments, at least a portion of at least some channel(s) have a substantially triangularly-shaped cross-section. In some embodiments, at least a portion of at least some channel(s) have a substantially triangularly-shaped cross-section having a single vertex at a base of the channel and having two other vertices at the surface of the base layer. Referring again to FIG. 24, in some embodiments, at least a portion of at least some of channels 102 have a substantially triangularly-shaped cross-section having a single vertex at a base of the channel and having two other vertices at the surface of the base layer.

As used herein, the term “triangular” is used to refer to a shape in which a triangle can be inscribed or circumscribed to approximate or equal the actual shape, and is not constrained purely to a triangle. For example, a triangular cross-section may comprise a non-zero curvature at one or more portions.

A triangular cross-section may comprise a wedge shape. As used herein, the term “wedge shape” will be known by those of ordinary skill in the art and refers to a shape having a thick end and tapering to a thin end. In some embodiments, a wedge shape has an axis of symmetry from the thick end to the thin end. For example, a wedge shape may have a thick end (e.g., surface opening of a channel) and taper to a thin end (e.g., base of a channel), and may have an axis of symmetry from the thick end to the thin end.

Additionally, in certain embodiments, substantially triangular cross-sections (i.e., “v-groove(s)”) may have a variety of aspect ratios. As used herein, the term “aspect ratio” for a v-groove refers to a height-to-width ratio. For example, in some embodiments, v-groove(s) may have an aspect ratio of less than or equal to 2, less than or equal to 1, or less than or equal to 0.5, and/or greater than or equal to 0.1, greater than or equal to 0.2, or greater than or equal to 0.3. Combinations of the above-referenced ranges are also possible (e.g., between or equal to 0.1 and 2, between or equal to 0.2 and 1). Other ranges are also possible.

In some embodiments, at least a portion of at least some channel(s) have a cross-section comprising a substantially triangular portion and a second portion opening into the substantially triangular portion and extending below the substantially triangular portion relative to the surface of the channel. In some embodiments, the second portion has a diameter (e.g., an average diameter) significantly smaller than an average diameter of the substantially triangular portion. Referring again to FIG. 24, in some embodiments, at least a portion of at least some of channels 102 have a cross-section comprising a substantially triangular portion 101 and a second portion 103 opening into substantially triangular portion 101 and extending below substantially triangular portion 101 relative to surface 105 of the channel, wherein second portion 103 has a diameter 107 significantly smaller than an average diameter 109 of substantially triangular portion 101. In some such cases, the second portion of a channel having a significantly smaller diameter than that of the average diameter of the substantially triangular portion of the channel can result in the substantially triangular portion being accessible to the roller of the apparatus and deformed portions of the surface layer, but the second portion being inaccessible to the roller and deformed portions of the surface layer. For example, referring again to FIG. 24, substantially triangular portion 101 of channel 102 is accessible to a roller (not pictured) and deformed portions of surface layer 106, while second portion 103 is inaccessible to the roller and deformed portions of surface layer 106, in accordance with certain embodiments. In some such cases, a seal with the surface layer 106 cannot be achieved in portions of the channel 102 having a second portion 103, because fluid can still move freely in second portion 103, even when surface layer 106 is deformed by a roller such that it fills substantially triangular portion 101 but not second portion 103. In some embodiments, a portion along a length of a channel may have both a substantially triangular portion and a second portion (“deep section”), while a different portion along the length of the channel has only the substantially triangular portion. In some such embodiments, when the apparatus (e.g., roller) engages with the portion having both a substantially triangular portion and a second portion (deep section), pump action is not started, because a seal with the surface layer is not achieved. However, as the apparatus engages along the length direction of the channel, when the apparatus deforms the surface layer at the portion of the channel having only a substantially triangular section, pump action begins because the lack of second portion (deep section) at that portion allows for a seal (and consequently a pressure differential) to be created. Therefore, in some cases, the presence and absence of deep sections along the length of the channels of the cartridge can allow for control of which portions of the channel are capable of undergoing pump action upon engagement with the apparatus.

The inclusion of such “deep sections” as second portions of at least some of the channels of the cartridge may contribute to any of a variety of potential benefits. For example, such deep sections (e.g., second portion 103) may, in some cases, contribute to a reduction in pump volume in peristaltic pumping processes. In some such cases, pump volume can be reduced by a factor of two or more for higher volume resolution. In some cases, such deep sections may also provide for a well-defined starting point for the pump volume that is not determined by where the roller lands on the channel. For example, the interface between a portion of a channel having both a substantially triangular portion and a second portion (deep section) and a portion of a channel having only a substantially triangular portion can, in some cases, be used as a well-defined starting point for the pump volume, because only fluid occupying the volume of the latter channel portion can be pumped. In some cases, where the rollers lands on the channel may have some error associated depending on any of a variety of factors, such as cartridge registration. The inclusion of deep sections may, in some cases, reduce or eliminate variations in pump volume associated with such error.

As used herein, an average diameter of a substantially triangular portion of a channel may be measured as an average over the z-axis from the vertex of the substantially triangular portion to the surface of the channel.

### EXAMPLES

Embodiments of the invention are further described with reference to the following examples, which are intended to be illustrative and not restrictive in nature. Although the examples below are described with reference to the separation of DNA oligonucleotides and methylated DNA oligonucleotides, embodiments of the present invention also have application in the purification and separation of other molecules having an affinity for agents immobilized within a medium, including other differentially modified molecules. Examples of such molecules include polypeptides or proteins, differentially modified polypeptides or proteins, differentially modified nucleic acids including differentially methylated DNA or RNA, or the like. Examples of agents that can be immobilized as probes in embodiments of the invention include DNA, RNA, antibodies, polypeptides, proteins, nucleic acid aptamers, and other agents with affinity for a molecule of interest.

### Example 1.0—Affinity SCODA with Single Base Mismatch

To verify the predicted temperature dependent mobility expressed in equation [23], experiments were performed to measure the response of target DNA velocity to changes in temperature. Initial experiments were done with 100 nucleotide oligonucleotides as target DNA. Oligonucleotides are single stranded so do not need to be denatured to interact with the affinity gel. The oligonucleotides are also sufficiently short that they have a negligible field dependent mobility. Longer nucleic acid molecules, e.g. greater than about 1000 nucleotides in length, may be difficult to separate based on sequence, as longer molecules have a tendency to focus in a non-sequence specific manner from the electrophoretic SCODA effect in embodiments using Joule heating provided by an electric field to provide the temperature gradient.

To perform these measurements a polyacrylamide gel (4% T, 2% C) in 1×TB (89 mM tris, 89 mM boric acid) with 0.2 M NaCl and 10 μM acrydite probe (SEQ ID NO. 1) oligo was cast in a one dimensional gel cassette containing only two access ports. Polymerization was initiated through the addition of 2 μl of 10% w/v APS and 0.2 μl TEMED per ml of gel.

Mobility measurements were performed on two different 100 nucleotide oligonucleotides differing by a single base containing sequences with a perfect match (PM) (SEQ ID NO. 2) to the probe and a single base mismatch (sbMM) (SEQ ID NO. 3). These target oligonucleotides were end labeled with either 6-FAM or Cy5 (IDT DNA). Probe and target sequences used for these experiments are shown in Table 3. The regions of the PM and sbMM target oligonucleotides that are complementary to the immobilized probe are shown in darker typeface than the other portions of these oligonucleotides. The position of the single base mismatch is underlined in the sbMM target sequence.

TABLE 3
Probe and target oligonucleotide
sequences used for sequence specific SCODA

| | Sequence |
|---|---|
| Probe | 5′ ACT GGC CGT CGT TTT ACT 3′ |
| (SEQ ID NO.: 1) | |
| PM Target | 5′ CGA TTA AGT TGA GTA ACG |
| (SEQ ID NO.: 2) | CCA CTA TTT TCA CAG TCA TAA |
| | CCA TGT AAA ACG ACG GCC AGT |
| | GAA TTA GCG ATG CAT ACC TTG |
| | GGA TCC TCT AGA ATG TAC C 3′ |
| sbMM Target | 5′ CGA TTA AGT TGA GTA ACG |
| (SEQ ID NO.: 3) | CCA CTA TTT TCA CAG TCA TAA |
| | CCA TGT AAA ACT ACG GCC AGT |
| | GAA TTA GCG ATG CAT ACC TTG |
| | GGA TCC TCT AGA ATG TAC C 3′ |

The probe sequence was chosen to be complementary to pUC19 for subsequent experiments with longer targets, discussed below. The 100 nucleotide targets contain a sequence complementary to the probe (perfect match: PM) or with a single base mismatch (sbMM) to the probe with flanking sequences to make up the 100 nucleotide length. The flanking sequences were designed to minimize the effects of secondary structure and self-hybridization. Initial sequences for the regions flanking the probe binding site were chosen at random. Folding and self-hybridization energies were then calculated using Mfold and the sequences were altered one base at a time to minimize these effects ensuring that the dominant interactions would be between target strands and the probe.

Table 4 shows the binding energies and melting temperatures for the sequences shown in Table 3 calculated using Mfold. The binding energy, ΔG, is given as ΔH-TΔS, where ΔH is the enthalpy and ΔS the entropy in units of kcal/mol and kcal/mol K respectively. The following parameter values were used for calculation of the values in Table 2: temperature=50° C., [Na+]=0.2 M, [Mg++]=0 M, strand concentration=10 μM. The largest T. for non probe-target hybridization is 23.9° C. and the greatest secondary structure T. is 38.1° C. Both of these values are far enough below the sbMM target-probe Tₘ that they are not expected to interfere target-probe interactions.

TABLE 4
Binding energies and melting temperatures for Table 3 sequences

| | Probe | PM Target | sbMM Target | Secondary |
|---|---|---|---|---|
| | (SEQ ID NO.: 1) | (SEQ ID NO.: 2) | (SEQ ID NO.: 3) | Structure |
| Probe | −35.4 + 0.1012 *T | −145.3 + 0.4039 * T | −126.8 + 0.3598 * T | −20.3 + 0.07049 * T |
| (SEQ ID NO.: 1) | Tm = 12.2° C. | Tm = 65.1° C. | Tm = 55.8° C. | Tm = 14.8° C. |
| PM Target | −145.3 + 0.4039 * T | −40.2 + 0.1124 * T | −40.2 + 0.1111 * T | −24.3 + 0.07808 * T |
| (SEQ ID No.: 2) | Tm = 65.1° C. | Tm = 23.9° C. | Tm = 20.9° C. | Tm = 38.1° C. |
| sbMM Target | −126.8 + 0.3598 * T | −40.2 + 0.1111 * T | −40.2 + 0.1124 * T | −24.3 + 0.07808 * T |
| (SEQ ID NO.: 3) | Tm = 55.8° C. | Tm = 20.9° C. | Tm = 23.9° C. | Tm = 38.1° C. |

To measure the velocity response as a function of temperature the fluorescently labeled target was first injected into the gel at high temperature (70° C.), and driven under a constant electric field into the imaging area of the gel. Once the injected band was visible the temperature of the spreader plate was dropped to 55° C. An electric field of 25 V/cm was applied to the gel cassette while the temperature was ramped from 40° C. to 70° C. at a rate of 0.5° C./min. Images of the gel were taken every 20 seconds. Image processing software written in LabView® (National Instruments, Austin Tex.) was used to determine the location of the center of the band in each image and this position data was then used to calculate velocity.

FIG. 11 shows a plot of target DNA mobility as a function of temperature. Using the values of ΔG for the probe and target sequences shown in Table 3, the velocity versus temperature curves were fit to equation [23] to determine the two free parameters: the mobility μ₀, and β a constant that depends on the kinetics of the hybridization reaction.

A fit of the data shown in FIG. 11 shows good agreement with the theory of migration presented above. Data for the mismatch mobility are shown as the curve on the left, and data for the perfect match mobility are shown as the curve on the right. The R² value for the PM fit and MM fits were 0.99551 and 0.99539 respectively. The separation between the perfect match and single base mismatch targets supports that there is an operating temperature where the focusing speed of the perfect match target is significantly greater than that of the mismatched target enabling separation of the two species through application of a DC bias field as illustrated in FIG. 4.

### Example 2.0—Selective Separation of Molecules Using Affinity SCODA

A 4% polyacrylamide gel containing 10 μM acrydite modified probe oligos (Integrated DNA Technologies, www.idtdna.com) was cast in a gel cassette to provide an affinity matrix.

Equimolar amounts of the perfect match and single base mismatch targets were injected into the affinity gel at 30° C. with an electric field of 100 V/cm applied across the gel such that both target molecules would be initially captured and immobilized at the gel buffer interface. The temperature was then increased to 70° C. and a constant electric field of 20 V/cm applied to the gel to move the target into the imaging area of the gel. The temperature was then dropped to 62° C. and a 108 V/cm SCODA focusing field superimposed over an 8 V/cm DC bias as shown in Table 2 was applied to the four source electrodes with a period of 5 seconds. The rotation direction of the SCODA focusing field was altered every period.

TABLE 5
Focusing plus bias potentials applied

| | Electrode A | Electrode B | Electrode C | Electrode D |
|---|---|---|---|---|
| Step 1 | −108 | 4 | 8 | 4 |
| Step 2 | 0 | −104 | 8 | 4 |
| Step 3 | 0 | 4 | −100 | 4 |
| Step 4 | 0 | 04 | 8 | −104 |

FIG. 12 shows images of concentration taken every 2 minutes. The perfect match target was tagged with 6-FAM and shown in green (leading bright spot which focuses to a tight spot), the mismatch target was tagged with Cy5 and is shown in red (trailing bright line that is washed from the gel). The camera gain was reduced on the green channel after the first image was taken. DNA was injected into the right side of the gel and focusing plus bias fields were applied. The perfect match target (green) experiences a drift velocity similar to that shown in FIG. 10A and moves towards a central focus location. The more weakly focusing mismatch target (red) experiences a velocity field similar to that shown in FIG. 10B and is pushed off the edge of the gel by the bias field. The direction of application of the applied washing field is indicated by the white arrow.

This experiment verifies the predictions of FIGS. 10A and 10B demonstrating that it is possible to generate two different velocity profiles for two DNA targets differing by only a single base enabling preferential focusing of the target with the higher binding energy to the gel. The images in FIG. 12 confirm that there are two distinct velocity profiles generated for the two different sequences of target DNA moving through an affinity matrix under the application of both a SCODA focusing field and a DC bias. A dispersive velocity field is generated for the single base mismatch target and a non dispersive velocity field is generated for the perfect match target. This example demonstrates that it is possible to efficiently enrich for targets with single base specificity, and optionally wash a non-desired target off of an affinity matrix, even if there is a large excess of mismatch target in the sample.

### Example 3.0—Optimization of Operating Conditions

Different parameters of the SCODA process may be optimized to achieve good sample enrichment at reasonable yields. In embodiments having immobilized (and negatively charged) DNA in the gel, a relatively high salinity running buffer was found to provide both efficient and stable focusing, as well as minimizing the time required to electrokinetically inject target DNA from an adjacent sample chamber into the SCODA gel.

### Example 3.1—Optimization of Buffer Salinity

Early attempts of measuring the temperature dependent mobility of molecules in an affinity gel as well as the first demonstrations of sequence specific SCODA were performed in buffers used for electrophoretic SCODA. These are typically standard electrophoresis buffers such as tris-borate EDTA (TBE), often diluted 4 to 6 fold to reduce the gel conductivity, enabling the application of high electric fields within thermal limitations imposed by Joule heating, resulting in shorter concentration times. Although it is possible to achieve sequence specific SCODA based concentration in a 1×TBE buffer (89 mM tris, 89 mM boric acid, 2 mM disodium EDTA), conditions can be further optimized for performance of sequence specific SCODA due to the relatively low concentration of dissociated ions at equilibrium in 1×TBE buffer. A low concentration of dissociated ions results in slow hybridization kinetics, exacerbates ionic depletion associated with immobilizing charges (oligonucleotide probes) in the gel, and increases the time required to electrokinetically inject target DNA into the gel. Calculations using 89 mM tris base and 89 mM boric acid, with a pKa of 9.24 for boric acid and a pKa of 8.3 for tris shows a concentration of 1.49 mM each of dissociated tris and dissociated boric acid in 1×TBE buffer.

### Example 3.2—Effect of Salt Concentration on DNA Hybridization

In embodiments used to separate nucleic acids, the presence of positive counter ions shields the electrostatic repulsion of negatively charged complementary strands of nucleic acid, resulting in increased rates of hybridization. For example, it is known that increasing the concentration of Na+ ions affects the rate of DNA hybridization in a non-linear manner (see Tsuruoka et al. Optimization of the rate of DNA hybridization and rapid detection of methicillin resistant Staphylococcus aureus DNA using fluorescence polarization. Journal of Biotechnology 1996; 48(3):201-208, which is incorporated by reference herein). The hybridization rate increases by about 10 fold when [NaCl] is increased from 10 mM to 1 M of [NaCl], with most of the gain achieved by the time one reaches about 200 mM. At low concentrations of positive counter ions, below about 10 mM, the rate of hybridization is more strongly dependent on salt concentration, roughly proportional to the cube of the salt concentration⁶. Theoretical calculations suggest that the total positive counter ion concentration of 1×TBE is around 5.5 mM (1.5 mM of dissociated tris, and 4 mM of Na+ from the disodium EDTA). At this ion concentration it was possible to achieve focusing however the slow hybridization rates resulted in weak focusing and large final focus spot sizes.

A slow rate of hybridization can lead to weak focusing through an increase in the phase lag between the changes in electric field and changes in mobility. Equation [16] describes the SCODA velocity as being proportional to cos(ϕ), where ϕ represents the phase lag between the mobility oscillations and the electric field oscillations. In the case of ssSCODA a phase lag can result from both a slow thermal response as well as from slow hybridization kinetics.

This phase lag results in slower focusing times and larger spot sizes since the final spot size is a balance between the inward SCODA-driven drift, and outward diffusion-driven drift. Faster focusing times are always desirable as this tends to reduce the overall time to enrich a target from a complex mixture. A smaller spot size is also desirable as it improves the ability to discriminate between different molecular species. As discussed above, when performing SCODA focusing under application of a DC bias, the final focus spot will be shifted off center by an amount that depends on both the mobility of the target and the speed of focusing, both of which depend on the strength of the interaction between the target and the gel bound probes. The amount of separation required to discriminate between two similar molecules when focusing under bias therefore depends on the final focus spot diameter. Smaller spot diameters should improve the ability to discriminate between two targets with similar affinity to the gel bound probes.

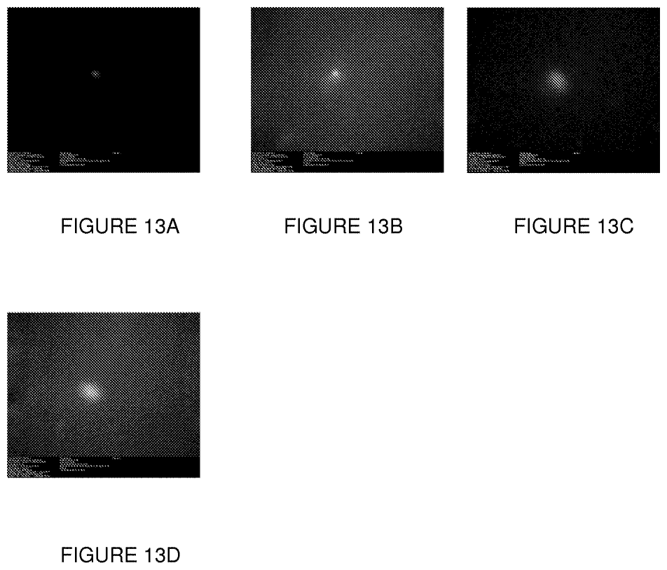

At the low rates of hybridization achieved with 1×TBE buffer, reliable focusing was only achievable with probe concentrations near 100 μM. Increasing the salt concentration from around 5 mM to 200 mM through the addition of NaCl, while keeping the probe concentration at 100 μM had the effect of reducing the final focus spot size as shown in FIGS. 13A-D. All images in FIGS. 13A-D were taken after a similar amount of focusing time (approximately 5 min), however the increased salinity resulted in increased Joule heating, which required a fourfold reduction of field strength to prevent boiling when focusing with 200 mM NaCl. Probe concentrations are 100 μM, 10 μM, 1 μM, and 100 μM, respectively in FIGS. 13A, 13B, 13C and 13D. The buffer used in FIGS. 13A, 13B and 13C was 1×TB with 0.2 M NaCl. The buffer used in FIG. 13D was 1×TBE. Focusing was not reliable at 10 μM and 1 μM probe in 1×TBE and these results are not shown. Under equivalent conditions in this example, addition of 200 mM NaCl to the gel also allowed for focusing of complementary targets at 100 fold lower probe concentrations.

Equation [30] states that the focusing speed is proportional to the electric field strength, so that fact that comparable focusing times are achieved with a fourfold reduction in electric field strength suggests that the field normalized focusing speed is considerably faster under high salinity conditions.

Although the total time for focusing was not reduced by the addition of 200 mM NaCl, focusing at lower electric field strength may be desirable in some embodiments because lower field strength can limit the degree of non-specific electrophoretic SCODA that may occur in an affinity matrix in some embodiments. For example, all target nucleic acid molecules will focus irrespective of their sequence in the affinity gels used for sequence specific SCODA in embodiments where the thermal gradient is established by an electric field due to electrophoretic SCODA. The speed of electrophoretic SCODA focusing increases with electric field, so decreasing the field strength will have the effect of reducing the non-specific SCODA focusing speed, allowing one to wash non-target DNA molecules from the gel more easily by applying a DC bias.

### Example 3.3—Ion Depletion and Bound Charges

The rate at which ions are depleted (or accumulated) at a boundary increases as the fraction of charges that are immobile increases. The 100 μM probe concentration required to achieve efficient concentration in 1×TBE results in 2 mM of bound negative charges within the gel when a 20 nucleotide probe is used, which is comparable to the total amount of dissolved negative ions within the gel (around 5.5 mM). This high proportion of bound charge can result in the formation of regions within the gel that become depleted of ions when a constant electric field is placed across the gel as it is during injection and during SCODA focusing under DC bias.

A high salinity running buffer can therefore help to minimize many of the ion depletion problems associated with immobilizing charges in an ssSCODA gel by enabling focusing at lower probe concentrations, as well as reducing the fraction of bound charges by adding additional free charges.

### Example 3.4—Denaturation of Double Stranded DNA

Target DNA will not interact with the gel immobilized probes unless it is single stranded. The simplest method for generating single stranded DNA from double stranded DNA is to boil samples prior to injection. One potential problem with this method is that samples can re-anneal prior to injection reducing the yield of the process, as the re-annealed double stranded targets will not interact with the probes and can be washed off of the gel by the bias field. Theoretical calculations show that the rate of renaturation of a sample will be proportional to the concentration of denatured single stranded DNA. Provided target concentration and sample salinity are both kept low, renaturation of the sample can be minimized.

To measure the effect of target concentration on renaturation and overall efficiency, fluorescently labeled double stranded PCR amplicons complementary to gel bound probes were diluted into a 250 μl volume containing about 2 mM NaCl and denatured by boiling for 5 min followed by cooling in an ice bath for 5 min. The sample was then placed in the sample chamber of a gel cassette, injected into a focusing gel and concentrated to the center of the gel. After concentration was complete the fluorescence of the final focus spot was measured, and compared to the fluorescence of the same quantity of target that was manually pipetted into the center of an empty gel cassette. This experiment was performed with 100 ng (2×10¹¹ copies) and 10 ng (2×10¹⁰ copies) of double stranded PCR amplicons. The 100 ng sample resulted in a yield of 40% and the 10 ng sample resulted in a yield of 80%. This example confirms that lower sample DNA concentration will result in higher yields.

### Example 3.5—Phase Lag Induced Rotation

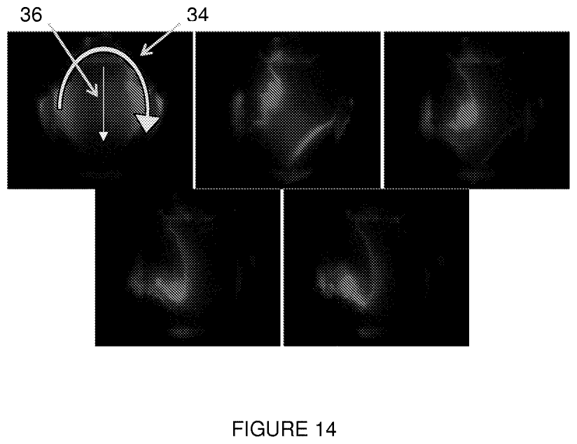

As discussed above, in embodiments in which there is a phase lag between the electric field oscillations and the mobility varying oscillations, a rotational component will be added to the velocity of molecules moving through the affinity matrix. An example of this problem is shown in FIG. 14. The targets shown in FIG. 14 focus weakly under SCODA fields and when a small bias is applied to wash them from the gel, the wash field and the rotational velocity induced by the SCODA fields sum to zero near the bottom left corner of the gel. This results in long wash times, and in extreme cases weak trapping of the contaminant fragments. The direction of rotation of the electric field used to produce SCODA focusing is indicated by arrow 34. The direction of the applied washing force is indicated by arrow 36.

To overcome this problem the direction of the field rotation can be altered periodically. In other examples described herein, the direction of the field rotation was altered every period. This results in much cleaner washing and focusing with minimal dead zones. This scheme was applied during focus and wash demonstrations described above and shown in FIG. 12, an example in which the mismatched target was cleanly washed from the gel without rotation. Under these conditions there is a reduced SCODA focusing velocity due to the phase lag, but there is not an additional rotational component of the SCODA velocity.

### Example 3.6—Effect of Secondary Structure

Secondary structure in the target DNA will decrease the rate of hybridization of the target to the immobilized probes. This will have the effect of reducing the focusing speed by increasing the phase lag described in equation [16]. The amount by which secondary structure decreases the hybridization rate depends on the details of the secondary structure. With a simple hairpin for example, both the length of the stem and the loop affect the hybridization rate⁹. For most practical applications of sequence specific SCODA, where one desires to enrich for a target molecule differing by a single base from contaminating background DNA, both target and background will have similar secondary structure. In this case the ability to discriminate between target and background will not be affected, only the overall process time. By increasing the immobilized probe concentration and the electric field rotation period one can compensate for the reduced hybridization rate.

There are potentially cases where secondary structure can have an impact on the ability to discriminate a target molecule from background molecules. It is possible for a single base difference between target and background to affect the secondary structure in such a way that background DNA has reduced secondary structure and increased hybridization rates compared to the target, and is the basis for single stranded conformation polymorphism (SSCP) mutation analysis. This effect has the potential to both reduce or enhance the ability to successfully enrich for target DNA, and care must be taken when designing target and probe sequences to minimize the effects of secondary structure. Once a target molecule has been chosen, the probe position can be moved around the mutation site. The length of the probe molecule can be adjusted. In some cases, oligonucleotides can be hybridized to sequences flanking the region where the probe anneals to further suppress secondary structure.

### Example 4.0—Quantitation of Sequence Specific SCODA Performance

The length dependence of the final focus location while focusing under DC bias was measured and shown to be independent of length for fragments ranging from 200 nt to 1000 nt in length; an important result, which implies that ssSCODA is capable of distinguishing nucleic acid targets by sequence alone without the need for ensuring that all targets are of a similar length. Measurements confirmed the ability to enrich for target sequences while rejecting contaminating sequences differing from the target by only a single base, and the ability to enrich for target DNA that differs only by a single methylated cytosine residue with respect to contaminating background DNA molecules.

### Example 4.1—Length Independence of Focusing

The ability to purify nucleic acids based on sequence alone, irrespective of fragment length, eliminates the need to ensure that all target fragments are of similar length prior to enrichment. The theory of sequence specific SCODA presented above predicts that sequence specific SCODA enrichment should be independent of target length. However, effects not modeled above may lead to length dependence, and experiments were therefore performed to confirm the length independence of sequence specific SCODA.

According to the theory of thermally driven sequence specific SCODA developed above, the final focus location under bias should not depend on the length of the target strands. Length dependence of the final focus location enters into this expression through the length dependence of the unimpeded mobility of the target μ₀. However, since both μ(Tₘ) and a are proportional to μ₀, the length dependence will cancel from this expression. The final focus location of a target concentrated with thermally driven ssSCODA should therefore not depend on the length of the target, even if a bias is present.

There are two potential sources of length dependence in the final focus location, not modeled above, which must also be considered: electrophoretic SCODA in embodiments where the temperature gradient is established by an electric field, and force based dissociation of probe target duplexes. DNA targets of sufficient length (>200 nucleotides) have a field dependent mobility in the polyacrylamide gels used for sequence specific SCODA, and will therefore experience a sequence independent focusing force when focusing fields are applied to the gel. The total focusing force experienced by a target molecule will therefore be the sum of the contributions from electrophoretic SCODA and sequence specific SCODA. Under electrophoretic SCODA, the focusing velocity tends to increase for longer molecules, while the DC velocity tends to decrease so that under bias the final focus location depends on length. The second potential source of length dependence in the final focus location is force based dissociation. The theory of affinity SCODA presented above assumed that probe-target dissociation was driven exclusively by thermal excitations. However it is possible to dissociate double stranded DNA with an applied force. Specifically, an external electric field pulling on the charged backbone of the target strand can be used to dissociate the probe-target duplex. The applied electric field will tend to reduce the free energy term ΔG in equation [22] by an amount equal to the energy gained by the charged molecule moving through the electric field. This force will be proportional to the length of the target DNA as there will be more charges present for the electric field to pull on for longer target molecules, so for a given electric field strength the rate of dissociation should increase with the length of the target.

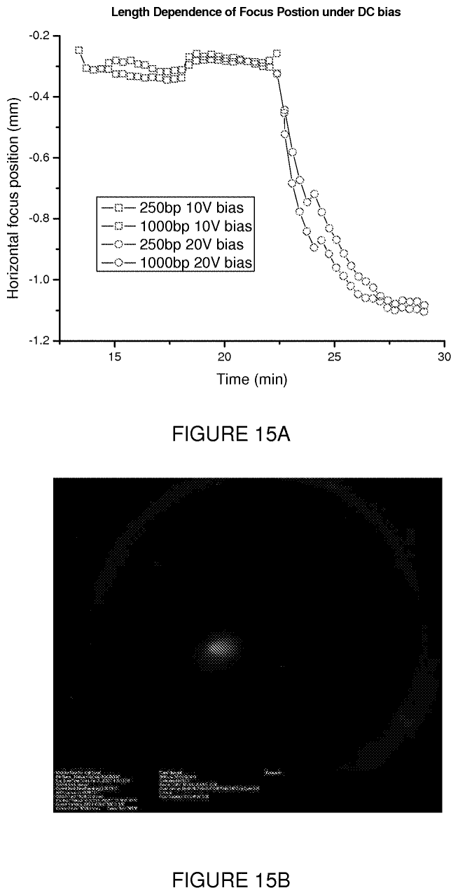

To measure whether or not these two effects contribute significantly to the length dependence of the final focus location, two different lengths of target DNA, each containing a sequence complementary to gel immobilized probes, were focused under bias and the final focus location measured and compared. The target DNA was created by PCR amplification of a region of pUC19 that contains a sequence complementary to the probe sequence in Table 3. Two reactions were performed with a common forward primer, and reverse primers were chosen to generate a 250 bp amplicon and a 1000 bp amplicon. The forward primers were fluorescently labeled with 6-FAM and Cy5 for the 250 bp and 1000 bp fragments respectively. The targets were injected into an affinity gel and focused to the center before applying a bias field. A bias field of 10 V/cm was superimposed over 120 V/cm focusing fields for 10 min at which point the bias was increased to 20 V/cm for an additional 7 min. Images of the gel were taken every 20 sec, with a 1 sec delay between the 6-FAM channel and the Cy5 channel. The field rotation period was 5 seconds. Images were post processed to determine the focus location of each fragment. FIGS. 15A and 15B show the focus location versus time for the 250 bp (green) and 1000 bp (red) fragments. FIG. 15B is an image of final focus of the two fragments at the end of the experiment.

There is a small difference in final location that can be attributed to the fact that the two images were not taken at the same phase in the SCODA cycle. This example shows that the final focus position does not depend on length. Thus, under these operating conditions electrophoretic SCODA focusing is much weaker than affinity SCODA focusing, and that affinity SCODA is driven largely by thermal dissociation rather than force-based dissociation. This result confirms that affinity SCODA is capable of distinguishing nucleic acid targets by sequence alone without the need for ensuring that all targets are of a similar length.

### Example 4.2—Single Base Mismatch Rejection Ratio

To demonstrate the specificity of ssSCODA with respect to rejection of sequences differing by a single base, different ratios of synthetic 100 nt target DNA containing either a perfect match (PM) or single base mismatch (sbMM) to a gel bound probe, were injected into an affinity gel. SCODA focusing in the presence of DC wash fields was performed to remove the excess sbMM DNA. The PM target sequence was labeled with 6-FAM and the sbMM with Cy5; after washing the sbMM target from the gel the amount of fluorescence at the focus location was quantified for each dye and compared to a calibration run. For the calibration run, equimolar amounts of 6-FAM labeled PM and Cy5 labeled PM target DNA were focused to the center of the gel and the fluorescence signal at the focus location was quantified on each channel. The ratio of the signal Cy5 channel to the signal on the 6-FAM channel measured during this calibration is therefore the signal ratio when the two dye molecules are present in equimolar concentrations. By comparing the fluorescence ratios after washing excess sbMM from the gel to the calibration run it was possible to determine the amount of sbMM DNA rejected from the gel by washing.

Samples containing target sequences shown in Table 3 were added to the sample chamber and an electric field of 50 V/cm was applied across the sample chamber at 45° C. to inject the sample into a gel containing 10 μM of immobilized probe. Once the sample was injected into the gel, the liquid in the sample chamber was replaced with clean buffer and SCODA focusing was performed with a superimposed DC wash field. A focusing field of 60 V/cm was combined with a DC wash field of 7 V/cm, the latter applied in the direction opposite to the injection field. It was found that this direction for the wash field led to complete rejection of the mismatched target DNA in the shortest amount of time. Table 6 below shows the amount of DNA injected into the gel for each experiment.

TABLE 6
List of targets run for measuring the rejection ratio of
affinity SCODA with respect to single base differences

| Run Description: | Cy5 Labeled Target | 6-FAM Labeled Target |
|---|---|---|
| 1:1 Calibration | 10 fmol PM | 10 fmol PM |
| 100:1 | 1 pmol sbMM | 10 fmol PM |
| 1,000:1 | 10 pmol sbMM | 10 fmol PM |
| 10,000:1 | 100 pmol sbMM | 10 fmol PM |
| 100,000:1 | 1 nmol sbMM | 10 fmol PM |

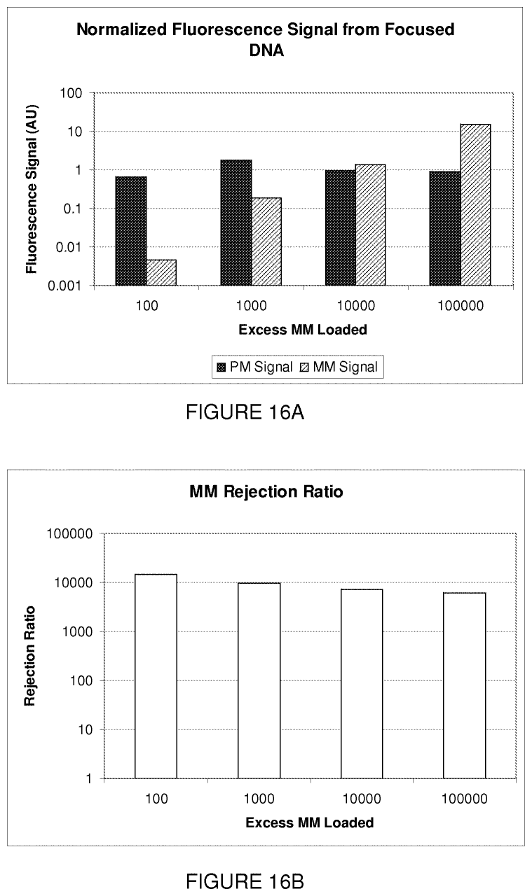

After the mismatched target had been washed from the gel, the focusing fields were turned off and the temperature of the gel was reduced to 25° C. prior to taking an image of the gel for quantification. It was important to ensure that all images used for quantification were taken at the same temperature, since Cy5 fluorescence is highly temperature dependent, with the fluorescence decreasing at higher temperatures. The ratio of fluorescence on the Cy5 and 6-FAM channels were compared to the 1:1 calibration run to determine the rejection ratio for each run. FIGS. 16A and 16B show the results of these experiments. Four different ratios of sbMM:PM were injected into a gel and focused under bias to remove excess sbMM. The PM DNA was tagged with 6-FAM and the sbMM DNA was tagged with Cy5. FIG. 16A shows the fluorescence signal from the final focus spot after excess sbMM DNA had been washed from the gel. The fluorescence signals are normalized to the fluorescence measured on an initial calibration run where a 1:1 ratio of PM-FAM:PMCy5 DNA was injected and focused to the center of the gel. FIG. 16B shows the rejection ratios calculated by dividing the initial ratio of sbMM:PM by the final ratio after washing.

It was found that rejection ratios of about 10,000 fold are achievable. However it should be noted that images taken during focusing and wash at high sbMM:PM ratios suggest that there were sbMM molecules with two distinct velocity profiles. Most of the mismatch target washed cleanly off of the gel while a small amount was captured at the focus. These final focus spots visible on the Cy5 channel likely consisted of Cy5 labeled targets that were incorrectly synthesized with the single base substitution error that gave them the PM sequence. The 10,000:1 rejection ratio measured here corresponds to estimates of oligonucleotide synthesis error rates with respect to single base substitutions, meaning that the mismatch molecule synthesized by IDT likely contains approximately 1 part in 10,000 perfect match molecules. This implies that the residual fluorescence detected on the Cy5 channel, rather than being unresolved mismatch may in fact be Cy5 labeled perfect match that has been enriched from the mismatch sample. Consequently the rejection ratio of ssSCODA may actually be higher than 10,000:1.

### Example 4.3—Mutation Enrichment for Clinically Relevant Mutation

The synthetic oligonucleotides used in the example above were purposely designed to maximize the difference in binding energy between the perfect match-probe duplex and the mismatch-probe duplex. The ability of affinity SCODA to enrich for biologically relevant sequences has also been demonstrated. In this example, cDNA was isolated from cell lines that contained either a wild type version of the EZH2 gene or a Y641N mutant, which has previously been shown to be implicated in B-cell non-Hodgkin Lymphoma. 460 bp regions of the EZH2 cDNA that contained the mutation site were PCR amplified using fluorescent primers in order to generate fluorescently tagged target molecules that could be visualized during concentration and washing. The difference in binding energy between the mutant-probe duplex and the wild type-probe duplex at 60° C. was 2.6 kcal/mol compared to 3.8 kcal/mol for the synthetic oligonucleotides used in the previous examples. This corresponds to a melting temperature difference of 5.2° C. for the mutant compared to the wild type. Table 7 shows the free energy of hybridization and melting temperature for the wild type and mutants to the probe sequence.

TABLE 7
Binding energy and melting temperatures
of EZH2 targets to the gel bound probe

| Target | Binding Energy |
|---|---|
| Wild Type | −161.9 + 0.4646 T |
| | Tm = 57.1° C. |
| Y641N Mutant | −175.2 + 0.4966 T |
| | Tm = 62.3° C. |

A 1:1 mixture of the two alleles were mixed together and separated with affinity SCODA. 30 ng of each target amplicon was added to 300 μl of 0.01× sequence specific SCODA running buffer. The target solution was immersed in a boiling water bath for 5 min then placed in an ice bath for 5 min prior to loading onto the gel cassette in order to denature the double stranded targets. The sample was injected with an injection current of 4 mA for 7 min at 55° C. Once injected, a focusing field of 150 V/cm with a 10 V/cm DC bias was applied at 55° C. for 20 min.

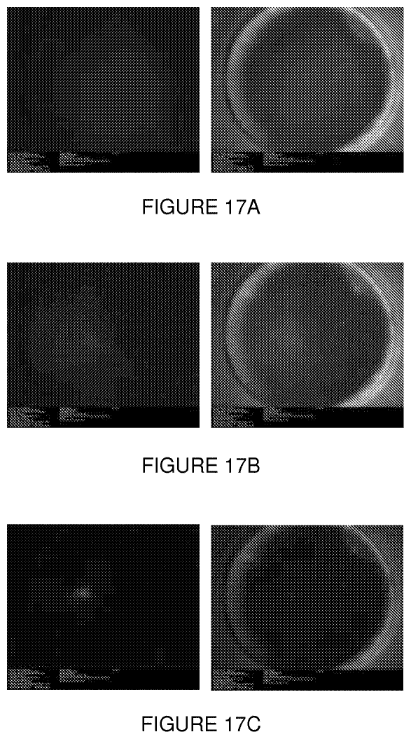

The result of this experiment is shown in FIGS. 17A, 17B and 17C. The behavior of these sequences is qualitatively similar to the higher Tₘ difference sequences shown in the above examples. The wild type (mismatch) target is completely washed from the gel (images on the right hand side of the figure) while the mutant (perfect match) is driven towards the center of the gel (images on the left hand side of the figure). In this case the efficiency of focusing was reduced as some of the target re-annealed forming double stranded DNA that did not interact with the gel bound probes.

The lower limit of detection with the optical system used was around 10 ng of singly labeled 460 bp DNA.

### Example 5.0—Methylation Enrichment

The ability of affinity SCODA based purification to selectively enrich for molecules with similar binding energies was demonstrated by enriching for methylated DNA in a mixed population of methylated and unmethylated targets with identical sequence.

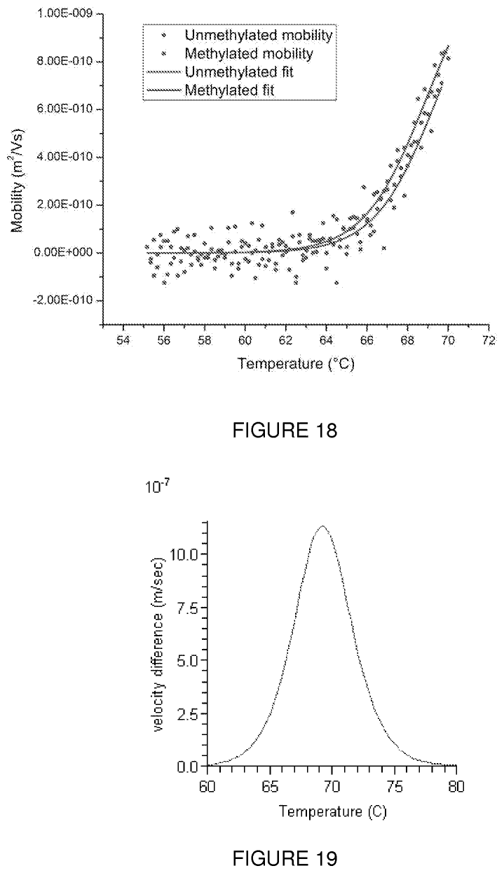

Fluorescently tagged PM oligonucleotides having the sequence set out in Table 3 (SEQ ID NO. 2) were synthesized by IDT with a single methylated cytosine residue within the capture probe region (residue 50 in the PM sequence of Table 3). DC mobility measurements of both the methylated and unmethylated PM strands were performed to generate velocity versus temperature curves as described above; this curve is shown in FIG. 18.

Fitting of these curves to equation [23] suggests that the difference in binding energy is around 0.19 kcal/mol at 69° C., which is about a third of the thermal energy FN1. The curve further suggests that separation of the two targets will be most effective at an operating temperature of around 69° C., where the two fragments have the greatest difference in mobility as shown in FIG. 19. In this example, the maximum value of this difference is at 69.5° C., which is the temperature for maximum separation while performing SCODA focusing under the application of a DC bias. At 69° C. k_{b}T=0.65 kcal/mol.

This temperature is slightly higher than that used in the above examples, and although it should result in better discrimination, focus times are longer as the higher temperature limits the maximum electric field strength one can operate at without boiling the gel.

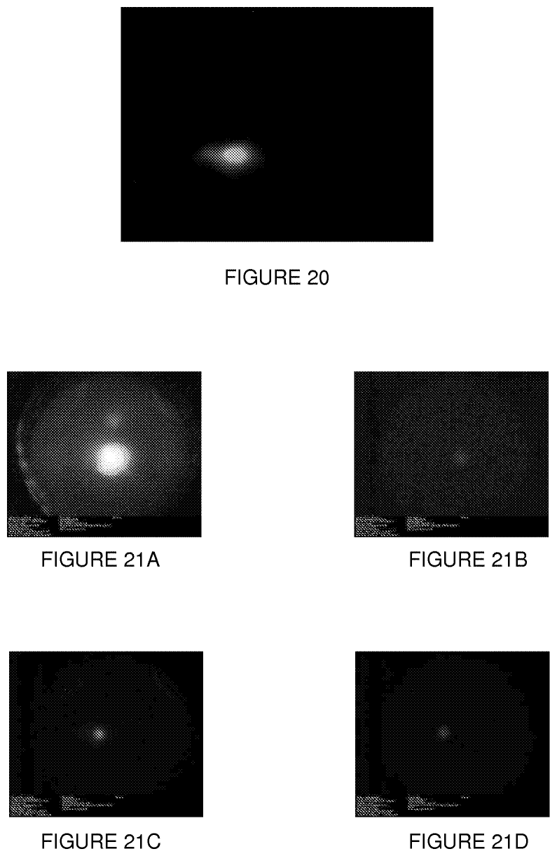

Initial focusing tests showed that it is possible to separate the two targets by performing affinity SCODA focusing with a superimposed DC bias. FIG. 20 shows the result of an experiment where equimolar ratios of methylated and unmethylated targets were injected into a gel, focused with a period of 5 sec at a focusing field strength of 75 V/cm and a bias of 14 V/cm at 69° C. Methylated targets were labeled with 6-FAM (green, spot on right) and unmethylated targets were labeled with Cy5 (red, spot on left). The experiment was repeated with the dyes switched, with identical results.

Achieving enrichment by completely washing the unmethylated target from the gel proved to be difficult using the same gel geometry for the above examples, as the gel buffer interface was obscured by the buffer wells preventing the use of visual feedback to control DC bias fields while attempting to wash the unmethylated target from the gel. To overcome this problem gels were cast in two steps: first a gel without probe oligonucleotides was cast in one of the arms of the gel and once the first gel had polymerized the remainder of the gel area was filled with gel containing probe oligonucleotides. The gels were cast such that the interface between the two was visible with the fluorescence imaging system. This system allowed for real time adjustments in the bias voltage so that the unmethylated target would enter the gel without immobilized probes and be quickly washed from the gel, while the methylated target could be retained in the focusing gel. FIGS. 21A-21D show the result of this experiment. FIGS. 21A and 21B show the results of an initial focus before washing unmethylated target from the gel for 10 pmol unmethylated DNA (FIG. 21A) and 0.1 pmol methylated DNA (FIG. 21B). FIGS. 21C and 21D show the results of a second focusing conducted after the unmethylated sequence had been washed from the gel for unmethylated and methylated target, respectively. All images were taken with the same gain and shutter settings.

In this experiment a 100 fold excess of unmethylated target was injected into the gel, focused to the center without any wash fields applied. The targets were then focused with a bias field to remove the unmethylated target, and finally focused to the center of the gel again for fluorescence quantification. Fluorescence quantification of these images indicates that the enrichment factor was 102 fold with losses of the methylated target during washing of 20%. This experiment was repeated with the dye molecules swapped (methylated Cy5 and unmethylated 6-FAM) with similar results.

### Example 6.0—Multiplexed Affinity SCODA

Two different oligonucleotide probes described above, one having affinity for EZH2 and one having affinity for pUC, were cast in a gel at a concentration of 10 μM each to provide an affinity matrix containing two different immobilized probes. A 100 nucleotide target sequence with affinity for the EZH2 probe and a theoretical melting temperature of 62.3° C. was labeled with Cy5. A 100 nucleotide target sequence with affinity for the pUC probe and a theoretical melting temperature of 70.1° C. was labeled with FAM. The theoretical difference in melting temperature between the two target molecules is 7.8° C.

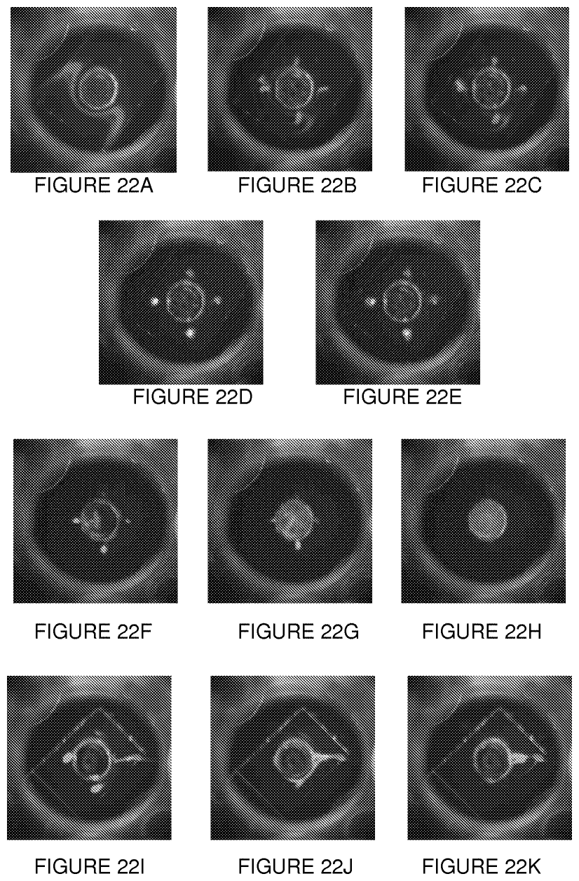

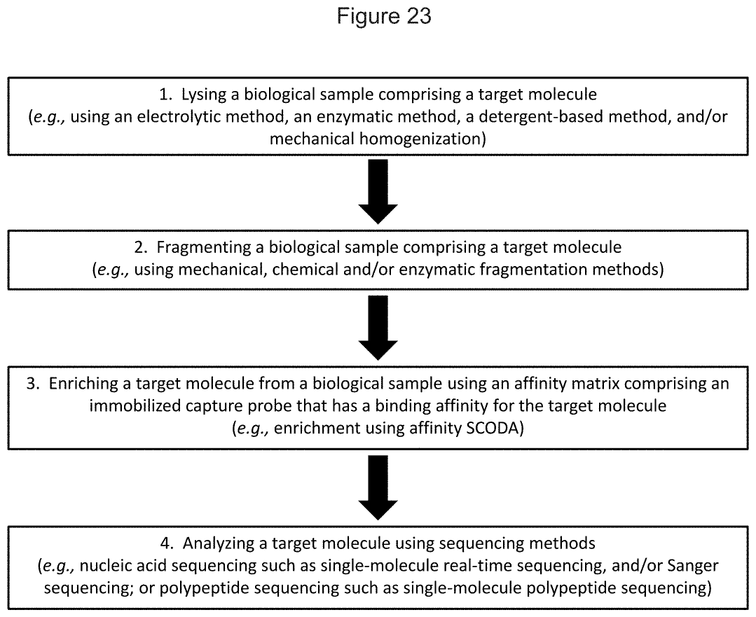

The target molecules were loaded on the affinity gel (FIG. 22A), and focusing was conducted with the temperature beneath the gel boat maintained at 55° C. (FIGS. 22B, focusing after two minutes, and 22C, after four minutes). The EZH2 target focused under these conditions (four red spots), while the pUC target focused only weakly under these conditions (three diffuse green spots visible on the gel). The central extraction well did not contain buffer during the initial portions of this experiment, resulting in the production of four focus spots, rather than a single central focus spot. The temperature beneath the gel was then increased to 62° C., a temperature increase of 7° C. (FIG. 22D, focusing two minutes after temperature increase, and 22E, after four minutes), resulting in the formation of four clear focus spots for the pUC target. The EZH2 target remained focused in four tight spots at this higher temperature.

The temperature beneath the gel was reduced to 55° C. and buffer was added to the central extraction well. Application of SCODA focusing fields at this temperature resulted in the EZH2 target being selectively concentrated into the central extraction well (diffuse red spot visible at the center of FIGS. 22F, 0.5 minutes, and 22G, 1 minute) while the pUC target remained largely focused in four spots outside the central extraction well. The temperature beneath the gel was increased to 62° C., a temperature increase of 7° C. Within two minutes, the pUC target had been focused into the central extraction well (FIG. 22H, diffuse red and green fluorescence visible at the center of the gel).

A second experiment was conducted under similar conditions as the first. After focusing the EZH2 target at 55° C. and the pUC target at 62° C. as described above, a DC washing bias was applied to the gel with the temperature beneath the gel maintained at 55° C. Under these conditions, the EZH2 target experienced a greater bias velocity than the pUC target. The focus spot for the EZH2 target shifted more quickly after the application of the bias field (red spot moving to the right of the gel in FIGS. 22I, 6 minutes after application of bias field, 22J, after 12 minutes, and 22K, after 18 minutes). The focus spot for the EZH2 target was also shifted a farther distance to the right within the gel. In contrast, the focus spot for the pUC target shifted more slowly (initial green focus spots still largely visible in FIG. 22I after 6 minutes, shifting to the right through FIGS. 22J, 12 minutes, and 22K, 18 minutes), and was not shifted as far to the right as the focus spot for the EZH2 target by the washing bias.

### Affinity SCODA Yield vs Purity

Because affinity SCODA relies on repeated interactions between target and probe to generate a non-dispersive velocity field for target molecules, while generating a dispersive field for contaminants (so long as a washing bias is applied), high specificity can be achieved without sacrificing yield. If one assumes that the final focus spot is Gaussian, which is justified by calculating the spot size for a radial velocity field balanced against diffusion, then the spot will extend all the way out to the edge of the gel. Here diffusion can drive targets off the gel where there is no restoring focusing force and an applied DC bias will sweep targets away from the gel where they will be lost. In this manner the losses for ssSCODA can scale with the amount of time one applies a wash field; however the images used to generate FIGS. 13A-13D indicate that in that example the focus spot has a full width half maximum (FWHM) of 300 μm and under bias it sits at approximately 1.0 mm from the gel center. If it is assumed that there is 10 fmol of target in the focus spot, then the concentration at the edge of the gel where a bias is applied is 1e-352 M; there are essentially zero target molecules present at the edges of the gel where they can be lost under DC bias. This implies that the rate at which losses accumulate due to an applied bias (i.e. washing step) is essentially zero. Although the desired target may be lost from the system in other ways, for example by adsorbing to the sample well prior to injection, running off the edge of the gel during injection, re-annealing before or during focusing (in the case of double stranded target molecules), or during extraction, all of these losses are decoupled from the purity of the purified target.

### Example 7.0—Use of a Sample Preparation Device

An automated sample preparation device of the disclosure was used to prepare a sample of DNA extracted from human blood.

The sample preparation device comprised a fluidics module (comprising a peristaltic pumping system), a temperature control module (to provide temperature and mechanical precision), a touch screen interface on the device that allowed the user to select any process-specific parameters (e.g., range of desired size of the nucleic acids, desired degree of homology for target molecule capture, etc.), and a lid that the user was able open in order to insert a sample preparation cartridge of the disclosure. The device was powered with a 1000-volt electrode supply. The sample preparation cartridge comprised thirteen discrete microfluidics channels (or pumping lanes) and was fabricated such that it could perform end-to-end sample preparation. The microfluidic channels were designed to manipulate reagents and the cartridge enabled, in automated succession: (1) Pipet introduction of combined sample lysis using lysis+lysis buffer and subsequent extraction of target DNA; (2) DNA purification; (3) DNA tagmentation using transposase Tn5 succeeded by DNA repair; (4) selection of DNA fragments of particular size range using nucleic acid capture probes and SCODA; and (5) DNA clean-up.

100 μL of whole human blood was mixed with lysis buffer and Proteinase K was incubated at 55° C. for 10 minutes then mixed with isopropanol; lysate mixture was subsequently added to a sample port in the sample preparation cartridge, the loaded cartridge was inserted into the sample preparation device, and DNA was extracted. The automated device, as described above, yielded 1.2 μg extracted DNA; 1 μg of that extracted DNA was further processed using the successive steps described above to generate 530 ng of a DNA library at a concentration of 6.5 nM. This purified DNA library produced by the sample preparation device was then subjected to sequencing using a glass sequencing chip.

As a control experiment, 100 μL of whole human blood (from the same sample as above) was manually processed to generate DNA library for sequencing using traditional DNA extraction and purification techniques.

The inventors found that sequencing data acquired using DNA library prepared using the automated sample preparation device was similar in quality (e.g., as assessed by average read length) relative to the sequencing data acquired using DNA manually prepared using traditional DNA extraction and purification techniques. As shown in Table 8, the automated device generated more total reads (72 total reads using automated process compared to 27 total reads using manual process) and greater read lengths (1989.0±760.1 base pair read lengths using automated process compared to 1132.1±324.5 base pair read lengths using manual process) than the manual process, with no significant difference observed between the processes in terms of accuracy and GC content of the resulting reads.

TABLE 8
Sequencing results from DNA libraries generated from whole human blood

| | | | Standard | | Standard | | Standard |
|---|---|---|---|---|---|---|---|
| | | Average | Deviation | Average | Deviation | Average | Deviation |
| | | Read | Read | Read | Read | GC | GC |
| | Total | Length | Length | Accuracy | Accuracy | content | content |
| | Reads | (bp) | (bp) | (%) | (%) | (%) | (%) |
| Manual | 27 | 1132.1 | 324.5 | 60.7% | 4.1% | 35.2% | 4.5% |
| process | | | | | | | |
| Automated | 72 | 1989.0 | 760.1 | 59.9% | 4.3% | 37.0% | 4.7% |
| process | | | | | | | |
| using | | | | | | | |
| Sample | | | | | | | |
| Preparation | | | | | | | |
| device of this | | | | | | | |
| disclosure | | | | | | | |

### Example 8.0—Use of a Sample Preparation Device to Enrich DNA for Sequencing

An automated sample preparation device of the disclosure was used to prepare a sample of DNA extracted from cultured E. coli cells.

The sample preparation device comprised a fluidics module (comprising a peristaltic pumping system), a temperature control module (to provide temperature and mechanical precision), a touch screen interface on the device that allowed the user to select any process-specific parameters (e.g., range of desired size of the nucleic acids, desired degree of homology for target molecule capture, etc.), and a lid that the user was able open in order to insert a sample preparation cartridge of the disclosure. The device was powered with a 1000-volt electrode supply. The sample preparation cartridge comprised thirteen discrete microfluidics channels (or pumping lanes) and was fabricated such that it could perform end-to-end sample preparation. The microfluidic channels were designed to manipulate reagents and the cartridge enabled, in automated succession: (1) Pipet introduction of combined sample+lysis buffer and subsequent extraction of target DNA; (2) DNA purification; (3) DNA tagmentation using transposase Tn5 succeeded by DNA repair; (4) selection of DNA fragments of particular size range using SCODA; and (5) DNA clean-up.

A sample of seven-hundred million E. coli cells from an overnight culture mixed with lysis buffer and Proteinase K was incubated at 55° C. for 10 minutes then mixed with isopropanol; lysate mixture was added to a sample port in the sample preparation cartridge, the loaded cartridge was inserted into the sample preparation device, and DNA was extracted. Automated processing continued to render the DNA into DNA library ready for sequencing with a brief pause for the user to add DNA Repair Enzyme and DNA Repair Buffer Mix to the cartridge just prior to the DNA Repair step. The automated device transported the DNA Repair Enzyme and DNA Repair Buffer Mix to the reaction location in the cartridge. The automated device, as described above, yielded 0.96 μg extracted DNA; subsequent automated steps generated 279 ng of a DNA library at a concentration of 2.89 nM.

As a control experiment, a sample of seven-hundred million E. coli cells (from the same sample as above) was manually processed to generate DNA using traditional DNA extraction and purification techniques. This manually prepared DNA was subjected to the same automated library preparation process on the automated device generating 199 ng of a DNA library at a concentration of 2.65 nM.

The purified DNA libraries produced by the sample preparation device were concentrated using Aline beads and then subjected to sequencing on a Pacific Biosciences® RSII DNA Sequencer.

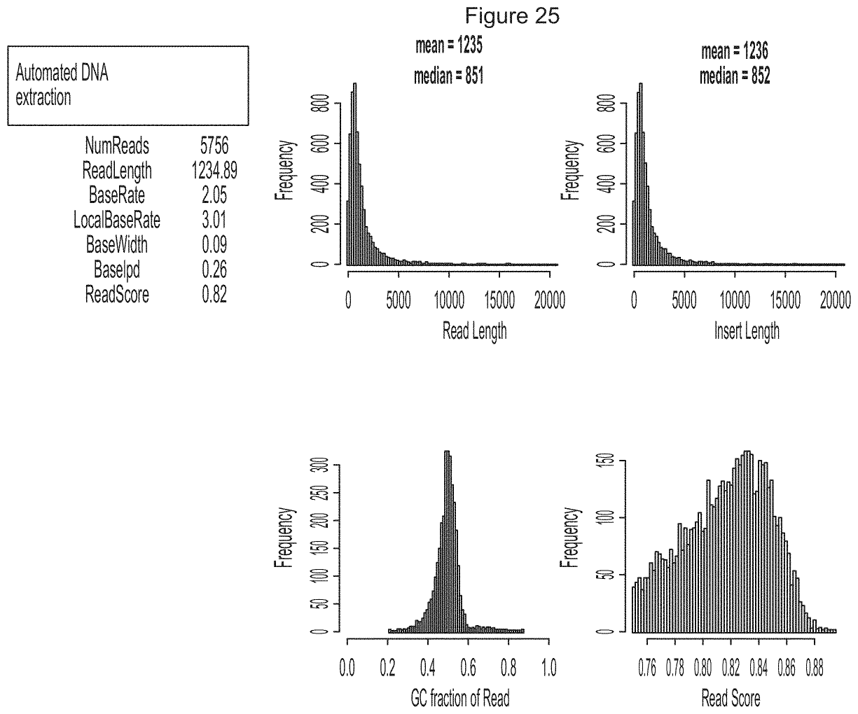

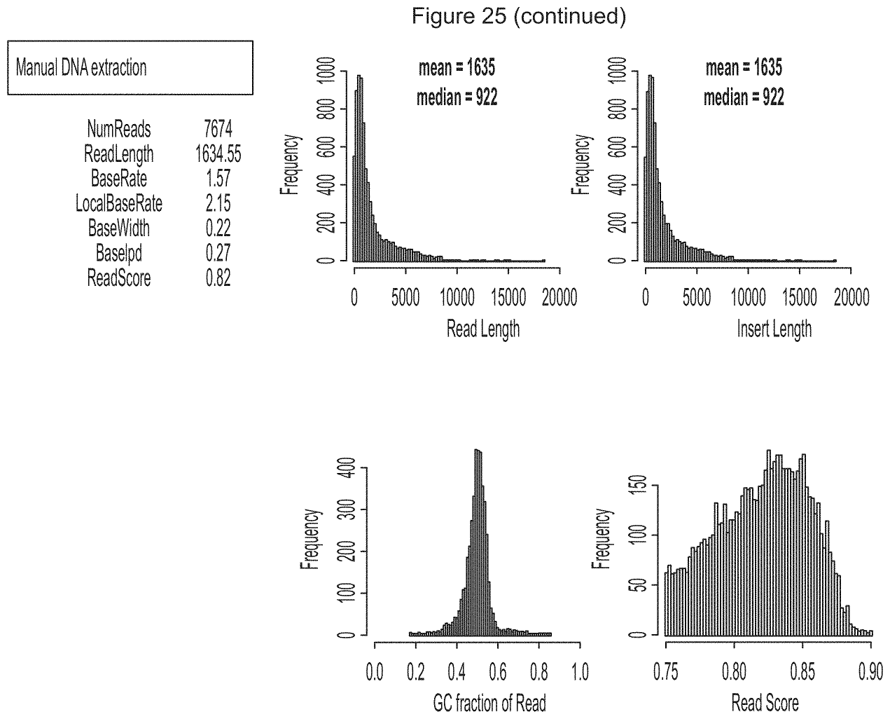

The inventors found that sequencing data acquired using DNA purified and prepared into library format using the automated sample preparation device generated sequencing reads that were slightly shorter in length, but similar in quality (as assessed by Rsq score) relative to the sequencing data acquired using DNA manually prepared with traditional DNA extraction and purification techniques followed by automated DNA library preparation (FIG. 25).

As shown in Table 9, the fully automated library generated reads with identical read quality (Rsq 0.82) to those generated with manual DNA extraction, with roughly equivalent read lengths (851 base average reads lengths versus 922 for manual).

TABLE 9
Sequencing results from DNA libraries generated from
E. coli cells extracted and purified via an Automated
Sample Preparation Device versus manually extracted
and purified DNA run on the same automated device

| Seq | | | | Median read | |
|---|---|---|---|---|---|
| name | Library | Treatment | Reads | length | RSq |
| C1856 | E2E | From lysate, | 5756 | 851 | 0.82 |
| | | E.coli library | | | |
| | | (Sample Prep device | | | |
| | | of this disclosure) | | | |
| C890 | MEAL | From purified DNA, | 7674 | 922 | 0.82 |
| | | E.coli library | | | |
| | | (Sample Prep device | | | |
| | | of this disclosure) | | | |

### Example 9.0—Use of a Sample Preparation Device to Enrich DNA for Sequencing

An automated sample preparation device of the disclosure was used to select DNA fragments of a particular size range using SCODA for a DNA library manually prepared from E. coli cultured cells.

Four micrograms of manually purified E. coli DNA was subjected to Tn5a tagmentation and then split into four separate samples consisting of 1 μg each. Selection of DNA fragments of a particular size was conducted separately by four different methods (1) Sage BluePippin with program to collect fragments from 3 kb to 10 kb in size, (2) Sage BluePippin with program to collect fragments greater in size than 4 kb to 10 kb, (3) manual Aline bead size selection with 0.45× bead addition, or (4) SCODA technology as in the automated sample preparation device (described in Example 8.0).

After size selection, each sample was separately prepared into DNA library and sequenced on a Pacific Biosciences® RSII DNA Sequencer.

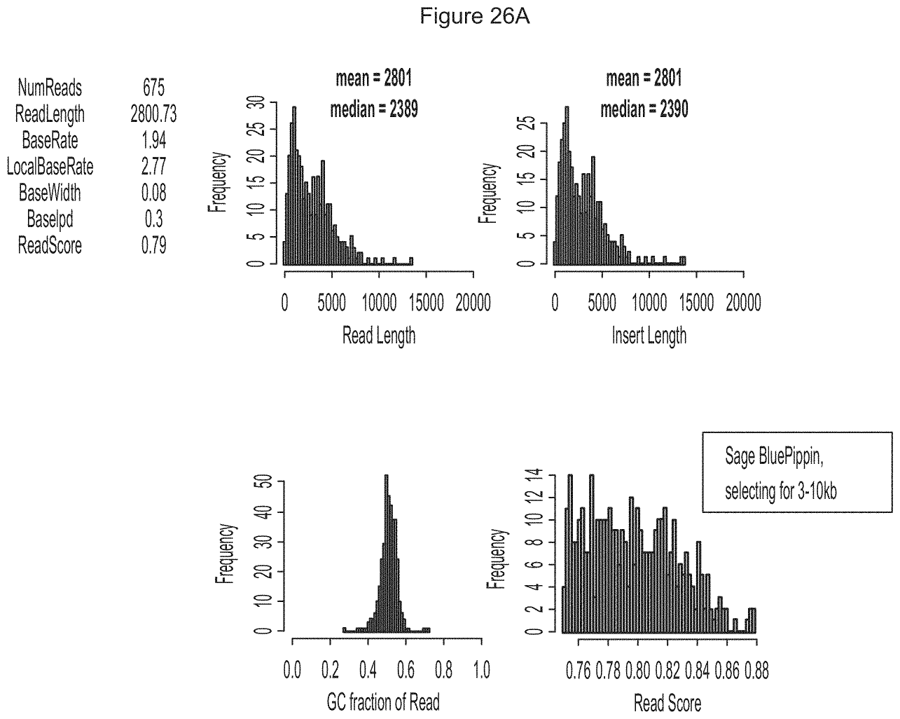

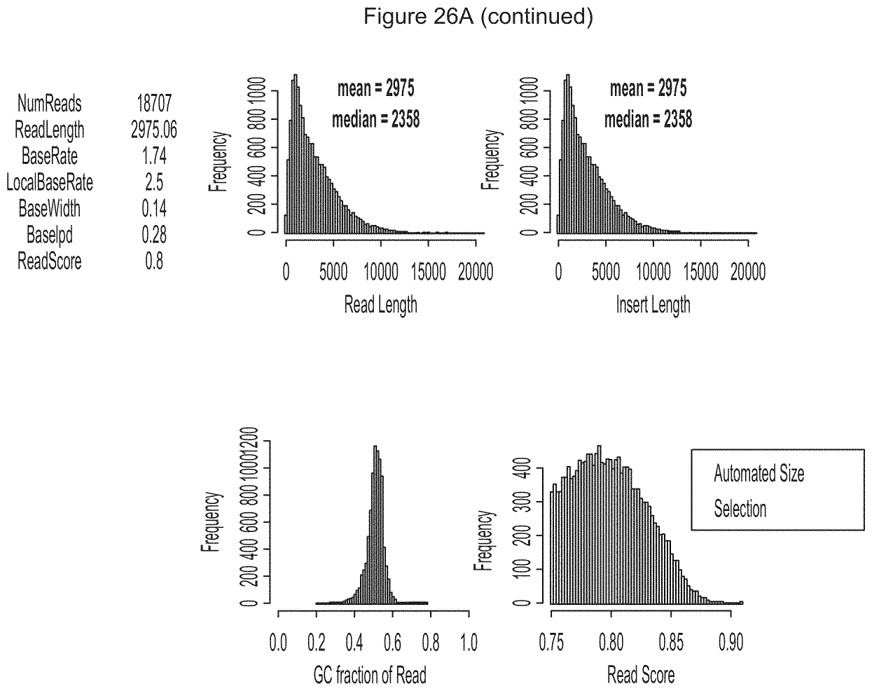

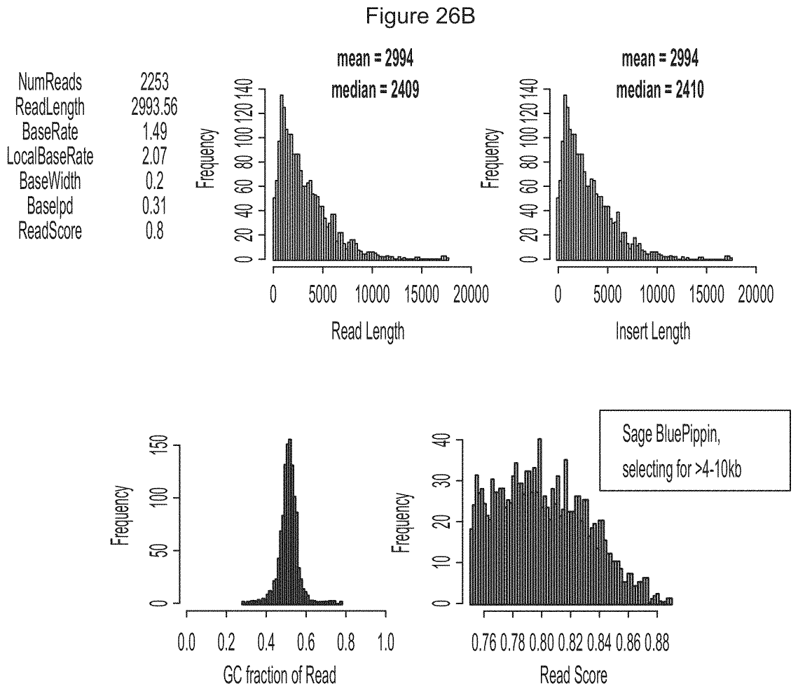

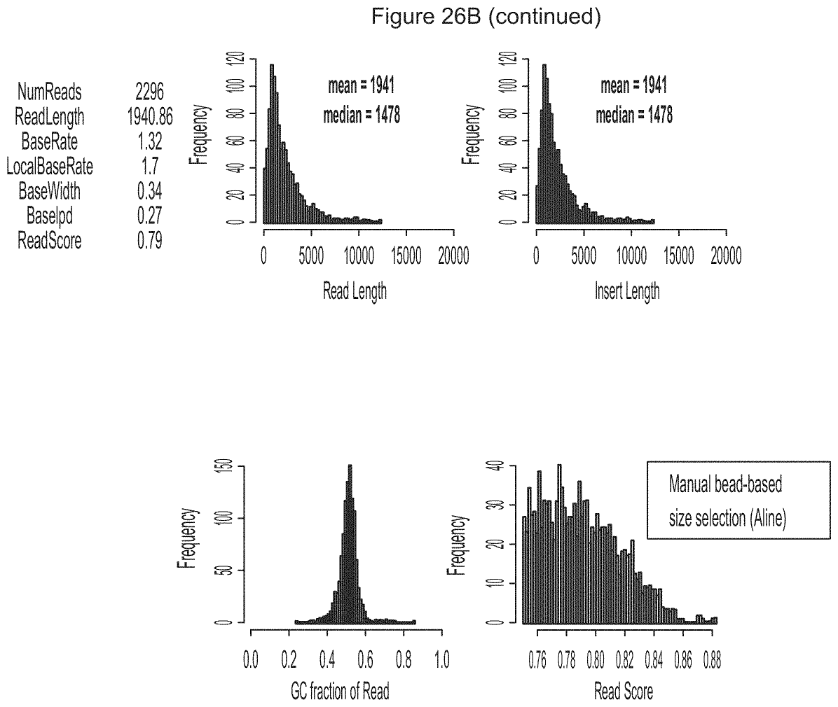

The inventors found that sequencing data acquired using DNA library size selection using the automated sample preparation device was superior to or equivalent to replicate DNA libraries selected for size by the standard manual bead-based process or the automated Sage BluePippin size selection method (FIG. 26).

As shown in Table 10 (below), the automated device generated read lengths longer than the manual size selection process and equivalent to the BluePippin methods with no significant difference observed among the processes in terms of accuracy and GC content of the resulting reads.

TABLE 10
Sequencing metrics from DNA libraries generated automated
size selection compared to those derived from samples
size selected by commercial and manual methods

| | | Median read |
|---|---|---|
| Size selection | Reads | length |
| Sage BluePippin, selecting for 3-10 kb range | 675 | 2389 |
| Sage BluePippin, selecting >4-10 kb high pass | 2253 | 2409 |
| Manual bead-based size selection (Aline) | 2296 | 1478 |
| Automated size selection (Sample Prep | 18707 | 2358 |
| device of this disclosure) | | |

### Additional Embodiments

Embodiments of the present invention relate to the induced movement of particles such as nucleic acids, proteins and other molecules through media such as gels and other matrices. Some embodiments provide methods and apparatus for selectively purifying, separating, concentrating and/or detecting particles of interest. Some embodiments provide methods and apparatus for selectively purifying, separating, concentrating and/or detecting differentially modified particles of interest. Some embodiments provide methods and apparatus for selectively purifying, separating, concentrating and/or detecting differentially methylated DNA. Some embodiments are used in fields such as epigenetics, oncology, or various fields of medicine. Some embodiments are used to detect fetal genetic disorders, biomarkers indicative of cancer or a risk of cancer, organ failure, disease states, infections, or the like.

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One embodiment provides a method for concentrating a molecule of interest from a biological sample. A biological sample is obtained from the subject and loaded on an affinity matrix. The affinity matrix has an immobilized affinity agent that has a first binding affinity for the molecule of interest and a second binding affinity for at least some of the other molecules in the biological sample. The first binding affinity is higher than the second binding affinity. Affinity SCODA is conducted to selectively concentrate the molecule of interest into a focus spot, wherein the concentration of the molecule of interest in the focus spot is increased relative to the concentration of the other molecules in the biological sample. The molecules may be nucleic acids. The molecule of interest may have the same sequence as at least some of the other molecules in the biological sample. The molecule of interest may be differentially modified as compared to at least some of the other molecules in the biological sample. The molecule of interest may be differentially methylated as compared to at least some of the other molecules in the biological sample. The biological sample may be maternal plasma and the molecule of interest may be fetal DNA that is differentially methylated as compared to maternal DNA. The biological sample may be a tissue sample and the molecule of interest may be a gene that is implicated in cancer that is differentially methylated as compared to the gene in a healthy subject.

One embodiment provides a method for separating a first molecule from a second molecule in a sample. An affinity matrix is provided with immobilized probes that bind to the first and second molecules. A binding energy between the first molecule and the probe is greater than a binding energy between the second molecule and the probe. A spatial gradient that is a mobility altering field that alters the affinity of the first and second molecules for the probe is provided within the affinity matrix. A driving field that effects motion of the molecules within the affinity matrix is applied. The orientation of both the spatial gradient and the driving field is varied over time to effect net motion of the first molecule towards a focus spot. A washing field is applied and is positioned to effect net motion of both the first and second molecules through the affinity matrix. The first and second molecules may be nucleic acids. The first and second molecules may be differentially modified. The first and second molecules may be differentially methylated. The first molecule may be fetal DNA and the second molecule may be maternal DNA that has the same sequence as the fetal DNA but is differentially methylated as compared to the fetal DNA. The first molecule and the second molecule may be a gene that is implicated in cancer, and the first molecule may be differentially methylated as compared to the second molecule.

One embodiment provides the use of a time-varying driving field in combination with a time-varying mobility altering field to separate first and second differentially methylated nucleic acid molecules, wherein the first and second nucleic acid molecules have the same DNA sequence. A time-varying driving field and a time-varying mobility altering field are applied to a matrix including an oligonucleotide probe that is at least partially complementary to said DNA sequence. The first nucleic acid molecule has a first binding energy to the oligonucleotide probe and the second nucleic acid molecule has a second binding energy to the oligonucleotide probe, and the first binding energy is higher than the second binding energy. The first nucleic acid molecules may be fetal DNA, the second nucleic acid molecules may be maternal DNA, and the first and second nucleic acid molecules may be obtained from a sample of maternal blood. The first and second nucleic acid molecules may be a gene that is implicated in a fetal disorder. The first and second molecules may be differentially methylated forms of a gene that is implicated in cancer. The first and second molecules may be obtained from a tissue sample of a subject. One embodiment provides the use of synchronous coefficient of drag alteration (SCODA) to detect the presence of a biomarker in a subject.

### FURTHER ASPECTS OF THE INVENTION

Aspects of the exemplary embodiments and examples described above may be combined in various combinations and subcombinations to yield further embodiments of the invention. To the extent that aspects of the exemplary embodiments and examples described above are not mutually exclusive, it is intended that all such combinations and subcombinations are within the scope of the present invention. It will be apparent to those of skill in the art that embodiments of the present invention include a number of aspects. Accordingly, the scope of the claims should not be limited by the preferred embodiments set forth in the description and examples, but should be given the broadest interpretation consistent with the description as a whole.

1. A device for analyzing a target molecule from a biological sample, the device comprising an automated sample preparation module connected to a sequencing module,
wherein the automated sample preparation module comprises a cartridge housing that is configured to receive a removable cartridge.

2. The device of claim 1, wherein the removable cartridge is a single-use cartridge or a multi-use cartridge.

3. The device of claim 1, wherein the removable cartridge is configured to receive the biological sample, optionally wherein the removable cartridge further comprises the biological sample.

4. The device of claim 1, wherein the automated sample preparation module is directly connected or indirectly connected to the sequencing module.

5. The device of claim 1, wherein the cartridge comprises one or more microfluidic channels configured to contain and/or transport a fluid used in a sample preparation process.

6. The device of claim 1, wherein the cartridge comprises one or more affinity matrices, wherein each affinity matrix comprises an immobilized capture probe that has a binding affinity for the target molecule.

7. The device of claim 1, wherein the biological sample is a blood, saliva, sputum, feces, urine or buccal swab sample.

8. The device of claim 1, wherein the target molecule is a target nucleic acid.

9. (canceled)

10. The device of claim 6, wherein the immobilized capture probe is an oligonucleotide capture probe, and wherein the oligonucleotide capture probe comprises a sequence that is at least partially complementary to a target nucleic acid.

11. The device of claim 10, wherein the oligonucleotide capture probe comprises a sequence that is at least 80%, 90% 95%, or 100% complementary to the target nucleic acid.

12. The device of claim 8, wherein the device produces target nucleic acids with an average sequencing read-length that is longer than an average sequencing read-length produced using control methods.

13. The device of claim 1, wherein the target molecule is a target protein.

14. The device of claim 6, wherein the immobilized capture probe is a protein capture probe that binds to a target protein.

15. The device of claim 14, wherein the protein capture probe is an aptamer or an antibody.

16. The device of claim 14, wherein the protein capture probe binds to the target protein with a binding affinity of 10⁻⁹ to 10⁻⁸ M, 10⁻⁸ to 10⁻⁷ M, 10⁻⁷ to 10⁻⁶ M, 10⁻⁶ to 10⁻⁵ M, 10⁻⁵ to 10⁻⁴ M, 10⁻⁴ to 10⁻³ M, or 10⁻³ to 10⁻² M.

17. The device of claim 1, wherein the sequencing module performs nucleic acid sequencing.

18. The device of claim 17, wherein the nucleic acid sequencing comprises single-molecule real-time sequencing, sequencing by synthesis, sequencing by ligation, nanopore sequencing, and/or Sanger sequencing.

19. The device of claim 1, wherein the sequencing module performs polypeptide sequencing.

20. The device of claim 19, wherein the polypeptide sequencing comprises edman degradation, single-molecule polypeptide sequencing, or mass spectroscopy.

21. (canceled)

22. A method of using the device of claim 1, the method comprising:
(i) lysing the biological sample in the sample preparation module;
(ii) fragmenting the lysed sample of (i) in the sample preparation module;
(iii) enriching the sample using an affinity matrix comprising an immobilized capture probe that has a binding affinity for the target molecule in the sample preparation module;
(iv) moving the target molecule from the in the automated sample preparation module to the sequencing module; and
(v) analyzing the target molecule in the sequencing module.


SEQUENCE LISTING (3 sequences)

SEQ ID NO: 1 - DNA, 18 bp, Artificial Sequence
  Synthetic Construct
actggccgtcgttttact

SEQ ID NO: 2 - DNA, 100 bp, Artificial Sequence
  Synthetic Construct
cgattaagttgagtaacgccactattttcacagtcataaccatgtaaaacgacggccagtgaattagcgatgcataccttgggatcctctagaatgtacc

SEQ ID NO: 3 - DNA, 100 bp, Artificial Sequence
  Synthetic Construct
cgattaagttgagtaacgccactattttcacagtcataaccatgtaaaactacggccagtgaattagcgatgcataccttgggatcctctagaatgtacc